(19) United States
(12) Patent Application Publication
(10) Pub. No.: US 2009/0163482 A1
(43) Pub. Date: Jun. 25, 2009

(54) TETRALINES ANTAGONISTS OF THE H-3 RECEPTOR

(76) Inventors: Stanton Furst McHardy, Helotes, TX (US); Vinod Dipak Parikh, Mystic, CT (US)

Correspondence Address: PFIZER INC;Steve T. Zelson, 150 EAST 42ND STREET, 5TH FLOOR - STOP 49, NEW YORK, NY, 10017-5612, US

(21) Appl. No.: 12/282,545

(22) PCT Filed: Mar. 1, 2007
(86) PCT No.: PCT/IB07/00536

§ 371 Date: Sep. 11, 2008

Related U.S. Application Data
(60) Provisional application No. 60/782,164, filed on Mar. 13, 2006.

(51) Int. Cl. A61K 31/541; C07D 295/14; A61K 31/40; C07D 417/12; A61P 11/00

(52) U.S. Cl. 514/227.8; 548/577; 514/429; 544/60

(57) ABSTRACT

This invention is directed to a compound of formula (I), as defined herein, or a pharmaceutically acceptable salt thereof, a pharmaceutical composition containing a compound of formula (I) a process of en preparation of a compound of formula (I), a method of treatment of a disorder or condition that may be treated by antagonizing histamine H3 receptors, the method comprising administering to a mammal in need of such treatment a compound of formula (I) as described above, and a method of treatment of a disorder or condition selected from the group consisting of depression, mood disorders, schizophrenia, anxiety disorders, Alzheimer's disease, attention-deficit hyperactivity disorder (ADHD), psychotic disorders, cognitive disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula (I) as described above.

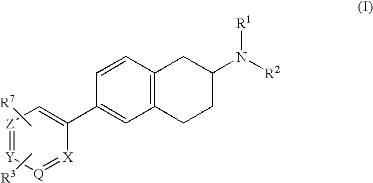

### BACKGROUND OF THE INVENTION

This invention is directed to compounds of formula I described herein, to a pharmaceutical composition comprising such compounds, and to methods of treatment of disorders or conditions that may be treated by antagonizing histamine-3 (H3) receptors using such compounds.

Histamine is a well-known mediator in hypersensitive reactions (e.g. allergies, hay fever, and asthma) that are commonly treated with antagonists of histamine or “antihistamines.” It has also been established that histamine receptors exist in at least two distinct types, referred to as H1 and H2 receptors.

A third histamine receptor (H3 receptor) is believed to play a role in neurotransmission in the central nervous system, where the H3 receptor is thought to be disposed presynaptically on histaminergic nerve endings (Nature, 302, S32-837 (1983)). The existence of the H3 receptor has been confirmed by the development of selective H3 receptor agonists and antagonists (Nature, 327, 117-123 (1987)) and has subsequently been shown to regulate the release of the neurotransmitters in both the central nervous system and peripheral organs, particularly the lungs, cardiovascular system and gastrointestinal tract.

A number of diseases or conditions may be treated with histamine-3 receptor ligands wherein the H3 ligand may be an antagonist, agonist or partial agonist, see: (Imamura et al., Circ. Res., (1996) 78, 475-481); (Imamura et. al., Circ. Res., (1996) 78, 863-869); (Lin et al., Brain Res. (1990) 523, 325-330); (Monti et al., Neuropsychopharmacology (1996) 15, 31 35); (Sakai, et al., Life Sci. (1991) 48, 2397-2404); (Mazurkiewiez-Kwilecki and Nsonwah, Can. J. Physiol. Pharmacol, (1989) 67, 75-78); (Panula, P. et al., Neuroscience (1998) 44, 465-481); (Wada et al., Trends in Neuroscience (1991) 14, 415); (Monti et al., Eur. J. Pharmacol. (1991) 205, 283); (Mazurkiewicz-Kwilecki and Nsonwah, Can. J. Physiol. Pharmacol. (1989) 67, 75-78); (Haas et al., Behav. Brain Res. (1995) 66, 41-44); (De Almeida and Izquierdo, Arch. Int. Pharmacodyn. (1986) 283, 193-198); (Karnel et al., Psychopharmacology (1990) 102, 312-318); (Kamei and Sakata, Japan. J. Pharmacol. (1991) 57, 437-482); (Schwartz et al., Psychopharmacology; The fourth Generation of Progress, Bloom and Kupfer (eds.), Raven Press, New York, (1995) 397); (Shaywitz et al., Psychopharmacology (1984) 82, 73-77); (Dumery and Blozovski, Exp. Brain Res. (1987) 67, 61-69); (Tedford et al., J. Pharmacol. Exp. Ther. (1995) 275, 598-604); (Tedford et al., Soc. Neurosci. Abstr. (1996) 22, 22); (Yokoyama et al., Eur. J. Pharmacol. (1993) 234, 129); (Yokoyama and Iinuma, CNS Drugs (1996) 5, 321); (Onodera et al., Prog. Neurobiol. (1994) 42, 685); (Leurs and Timmerman, Prog. Drug Res. (1992) 39, 127); (The Histamine H3 Receptor, Leurs and Timmerman (ed.), Elsevier Science, Amsterdam, The Netherlands (1998); (Leurs et al., Trends in Pharm. Sci. (1998) 19, 177-183); (Phillips et al., Annual Reports in Medicinal Chemistry (1998) 33, 31-40); (Matsubara et al., Eur. J. Pharmacol. (1992) 224, 145); (Rouleau et al., J. Pharmacol. Exp. Ther. (1997) 281, 1085); (Adam Szelag, “Role of histamine H3-receptors in the proliferation of neoplastic cells in vitro”, Med. Sci. Monit., 4(5): 747-755, (1998)); (Fitzsimons, C., H. Duran, F. Labombarda, B. Molinari and E. Rivera, “Histamine receptors signalling in epidermal tumor cell lines with H-ras gene alterations”, Inflammation Res., 47 (Suppl. 1): S50-651, (1998)); (R. Leurs, R. C. Volling a and H. Timmerman, “The medicinal chemistry and therapeutic potentials of ligand of the histamine H3 receptor”, Progress in Drug Research 45: 170-165, (1995)); (R. Levi and N. C. E. Smith, “Histamine H3-receptors: A new frontier in myocardial ischemia”, J. Pharm. Exp. Ther., 292: 825-830, (2000)); (Hatta, E., K Yasuda and R. Levi, “Activation of histamine H3 receptors inhibits carrier-mediated norepinephrine release in a human model of protracted myocardial ischemia”, J. Pharm. Exp. Ther., 283: 494-500, (1997); (H. Yokoyama and K. Iinuma, “Histamine and Seizures: Implications for the treatment of epilepsy”, CNS Drugs, 5(5); 321-330, (1995)); (K. Hurukami, H. Yokoyama, K. Onodera, K. Iinuma and T. Watanabe, AQ-0 145, “A newly developed histamine H3 antagonist, decreased seizure susceptibility of electrically induced convulsions in mice”, Meth. Find. Exp. Clin. Pharmacol., 17(C): 70-73, (1995); (Delaunois A., Gustin P., Garbarg M., and Ansay M., “Modulation of acetylcholine, capsaicin and substance P effects by histamine H3 receptors in isolated perfused rabbit lungs”, European Journal of Pharmacology 277(2-3):243-50, (1995)); and (Dimitriadou, et al., “Functional relationship between mast cells and C— sensitive nerve fibres evidenced by histamine H3-receptor modulation in rat lung and spleen”, Clinical Science 87(2):15163, (1994). Such diseases or conditions include cardiovascular disorders such as acute myocardial infarction; memory processes, dementia and cognitive disorders such as Alzheimer's disease and attention-deficit hyperactivity disorder; neurological disorders such as Parkinson's disease, schizophrenia, depression, epilepsy, and seizures or convulsions; cancer such as cutaneous carcinoma, medullary thyroid carcinoma and, melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

H3 receptor antagonists have also been previously described in, for example, WO 03/050099, WO 02/0769252, WO 02/12224, and U.S. Patent Publication No. 2005/0171181 A1. The histamine H3 receptor (H3R) regulates the release of histamine and other neurotransmitters, including serotonin and acetylcholine. H3R is relatively neuron specific and inhibits the release of certain monoamines such as histamine. Selective antagonism of H3R receptors raises brain histamine levels and inhibits such activities as food consumption while minimizing non-specific peripheral consequences. Antagonists of the receptor increase synthesis and release of cerebral histamine and other monoamines. By this mechanism, they induce a prolonged wakefulness, improved cognitive function, reduction in food intake and normalization of vestibular reflexes. Accordingly, the receptor is an important target for new therapeutics in Alzheimer disease, mood and attention adjustments, including attention deficit hyperactive disorder (ADHD), cognitive deficiencies, obesity, dizziness, schizophrenia, epilepsy, sleeping disorders, narcolepsy and motion sickness, and various forms of anxiety.

The majority of histamine H3 receptor antagonists to date resemble histamine in possessing an imidazole ring that may be substituted, as described, for example, in WO 96/38142 Non-imidazole neuroactive compounds such as beta histamines (Arrang, Eur. J. Pharm. 1985, 111:72-84) demonstrated some histamine H3 receptor activity but with poor potency. EP 978512 and EP 0982300A2 disclose non-imidazole alkyamines as histamine H3 receptor antagonists. WO 02/12224 (Ortho McNeil Pharmaceuticals) describes non-imidazole bicyclic derivatives as histamine H3 receptor ligands. Other receptor antagonists have been described in WO 02/32893 and WO 02/06233.

This invention is directed to histamine-3 (H3) receptor antagonists of the invention useful for treating the conditions listed in the preceding paragraphs. The compounds of this invention are highly selective for the H3 receptor (vs. other histamine receptors), and possess remarkable drug disposition properties (pharmacokinetics). In particular, the compounds of this invention selectively distinguish H3R from the other receptor subtypes H1R, H2R. In view of the increased level of interest in histamine H3 receptor agonists, inverse agonists and antagonists in the art, novel compounds that interact with the histamine H3 receptor would be a highly desirable contribution to the art. The present invention provides such a contribution to the art being based on the finding that a novel class of tetraline amines has a high and specific affinity to the histamine H3 receptor.

### SUMMARY OF THE INVENTION

This invention is directed to a compound of formula I:

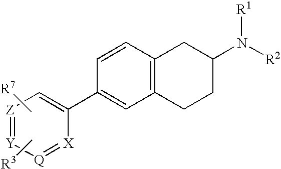

or a pharmaceutically acceptable salt thereof, wherein

Z, Y, Q, X are independently nitrogen or carbon;

R¹ and R² are independently hydrogen, (C₁-C₈)alkyl optionally substituted with 1 to 4 halogens, or (C₃-C₇)cycloalkyl-(C₀-C₄)alkyl, wherein each (C₀-C₄) is optionally substituted with one to four (C₁-C₄)alkyl;

or optionally R¹ and R², together with the nitrogen to which they are attached, form a 4 to 7-membered heterocycloalkyl ring, wherein one of the carbons of said heterocycloalkyl ring that is separated by at least two atoms from said nitrogen in said heterocycloalkyl ring is optionally replaced by O, S, NR⁶, or C═O, wherein R⁶ is hydrogen, (C₁-C₃)alkyl, or —C(═O)(C₁-C₃)alkyl; and wherein said heterocycloalkyl ring is optionally substituted with halo, one or two (C₁-C₄)alkyl, phenyl, (C₁-C₈)alkyl optionally substituted with 1 to 4 halogens, or (C₃-C₇)cycloalkyl-(C₀-C₄)alkyl, and wherein each (C₀-C₄)alkyl is optionally substituted with one to four (C₁-C₄)alkyl;

R₃ is hydrogen, (C₁-C₆)alkyl, (C₁-C₆)alkoxy, halo, 5 to 6-membered aryl, 5 to 6-membered heteroaryl, hydroxyl, methylene hydroxyl, —(C═O)NR₄R₅, and S(O)ₚ(C₁-C₄)alkyl, where p is 1 or 2;
wherein R⁴ and R⁵ are independently selected from the group consisting of hydrogen,
(C₁-C₈)alkyl optionally substituted with 1 to 4 halogens;
(C₁-C₄)alkyl group optionally substituted with a substituent selected from the group consisting of OH, 1 to 4 (C₁-C₄)alkyl, (C₃-C₇)cycloalkyl, (C₁-C₄)dialkylamino, (C₆-C₁₄)aryl optionally substituted with a halogen and optionally substituted with (C₆-C₁₀)aryloxy optionally substituted with 1 to 2 halogens, and 5 to 10-membered heteroaryl optionally substituted with a (C₆-C₁₀)aryl group and optionally substituted with 1 to 3 (C₁-C₄)alkyl groups;
(C₃-C₇)cycloalkyl;
(C₆-C₁₄)aryl;
-(C₂-C₈)alkyl-O—(C₁-C₃)alkyl optionally substituted with (C₁-C₃)alkyl;
—(C₁-C₃)alkyl-C(═O)O—(C₁-C₃)alkyl;
3-8-membered heterocycloalkyl optionally substituted with one or more (C₁-C₄)alkyl-carbonyl groups;
(C₆-C₁₀)arylsulfonyl optionally substituted with one or more (C₁-C₂)alkyl;
5-10-membered heteroaryl; and
(C₆-C₁₄)aryl-(C₀-C₄)alkylene-O—(C₀-C₄)alkyl, wherein each (C₀-C₄)alkyl and each (C₀-C₄)alkylene is optionally substituted with 1 to 4 (C₁-C₄ alkyl);
or optionally R⁴ and R⁵, together with the nitrogen to which they attached, form a 4 to 6-membered heterocyclic ring, wherein one of the carbons of said heterocyclic ring that is separated by at least two atoms from said nitrogen in said heterocyclic ring is optionally replaced by O or NR⁶, wherein R^{r} is hydrogen, (C₁-C₃)alkyl, or —C(═O)(C₁-C₃)alkyl; and wherein said heterocyclic ring is optionally substituted with halo, (C₁-C₃)alkyl, or hydroxyl;

R⁷ is hydrogen;

or optionally R³ and R⁷ together with two adjacent atoms in the ring comprising Z, Y, Q and X to which they are attached, form a 5 or 6-membered heterocyclic ring; wherein one of the carbons of said heterocyclic ring that is separated by at least two atoms from said nitrogen in said heterocyclic ring is optionally replaced by O or NR⁸; wherein R⁸ is hydrogen or (C₁-C₃)alkyl.

A preferred embodiment includes compounds of formula I, wherein, the invention is directed to a compound of formula I, wherein Y and X are carbon; Q and Z are carbon or nitrogen; R⁷ is hydrogen; R¹ and R² together form a 5-membered heterocycloalkyl ring, optionally substituted with (C₁-C₄)alkyl; and R³ is selected from the group consisting of methoxy, —(C═O)NR₄R₆, and S(O)ₚ(C₁-C₄)alkyl; wherein R⁴ and R⁵ are independently hydrogen or (C₁-C₄)alkyl; and wherein p is 1 or 2.

Another preferred embodiment includes compounds of formula I, wherein Z, Y, X, and Q are carbon; R¹ and R² together with the nitrogen to which they are attached form a 5 membered heterocycloalkyl ring optionally substituted with methyl;
R⁷ is hydrogen;
R³ is —(C═O)NR₄R₅;
wherein R⁴ and R⁵ are independently selected from the group consisting of hydrogen;
(C₁-C₈)alkyl optionally substituted with 1 to 4 halogens;
(C₁-C₄)alkyl group optionally substituted with a substituent selected from the group consisting of OH, 1 to 4 (C₁-C₄)alkyl, (C₃-C₇)cycloalkyl, (C₁-C₄)dialkylamino, (C₆-C₁₄)aryl optionally substituted with a halogen and optionally substituted with (C₆-C₁₀)aryloxy optionally substituted with 1 to 2 halogens, and 5 to 10-membered heteroaryl optionally substituted with a (C₈-C₁₀)aryl group and optionally substituted with 1 to 3 (C₁-C₄)alkyl groups;
(C₃-C₇)cycloalkyl;
(C₆-C₁₄)aryl;
-(C₂-C₃)alkyl-O—(C₁-C₃)alkyl optionally substituted with (C₁-C₃)alkyl;
-(C₁-C₃)alkyl-C(═O)O—(C₁-C₃)alkyl;
3-8-membered heterocycloalkyl optionally substituted with one or more (C₁-C₄)alkyl-carbonyl groups;
(C₆-C₁₀)arylsulfonyl optionally substituted with one or more (C₁-C₂)alkyl;
5-10-membered heteroaryl; and
(C₆-C₁₄)aryl-(C₀-C₄)alkylene-O—(C₀-C₄)alkyl, wherein each (C₀-C₄)alkyl and each (C₀-C₄)alkylene is optionally substituted with 1 to 4 (C₁-C₄ alkyl);
or optionally R⁴ and R⁵, together with the nitrogen to which they attached, form a 4 to 6-membered heterocyclic ring, wherein one of the carbons of said heterocycloalkyl ring that is separated by at least two atoms from said nitrogen in said heterocycloalkyl ring is optionally replaced by C or NR⁸, wherein R⁸ is hydrogen or (C₁-C₃)alkyl; and wherein said heterocycloalkyl ring is optionally substituted with halo, (C₁-C₃)alkyl, or hydroxyl.

Another preferred embodiment includes compounds of formula I, wherein Z, Y, X, and Q are carbon;

R¹ and R² together with the nitrogen to which they are attached form a 5-membered heterocycloalkyl ring optionally substituted with methyl;

R⁷ is hydrogen;

R³ is —(C═O)NR₄R₅; wherein R⁴ and R⁶ are independently selected from the group consisting of hydrogen, (C₁-C₅)alkyl, (C₃-C₅)cycloalkyl.

Another preferred embodiment includes compounds of formula I, wherein Z, Y, X, and Q are carbon;

R¹ and R² together with the nitrogen to which they are attached form a 5-membered heterocycloalkyl ring optionally substituted with methyl;

R⁷ is hydrogen;

R³ is —(C═O)NR₄R⁵; wherein R⁴ and R⁵, together with the nitrogen to which they are attached, form a 4 to 6-membered heterocycloalkyl ring, and wherein said heterocycloalkyl ring is optionally substituted with halo, hydroxy, or (C₁-C₅)alkyl.

Another preferred embodiment includes compounds of formula I, wherein X, Y, Z are carbon;

Q is nitrogen;

R¹ and R² together with the nitrogen to which they are attached form a 5-membered heterocyclic ring optionally substituted with methyl; R³ is selected from the group, consisting of hydrogen, methyl, ethyl, methoxy, and ethoxy; and R⁷ is hydrogen.

Another preferred embodiment includes compounds of formula I, wherein X is carbon; Z and Q are nitrogen; R³ is selected from the group consisting of hydrogen, methyl, ethyl, methoxy, and ethoxy; and R⁷ is hydrogen.

Another preferred embodiment includes compounds of formula I, wherein said compound has the following structure:

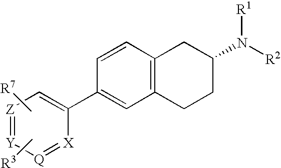

Another preferred embodiment includes compounds of formula I, wherein said compound has the following structure:

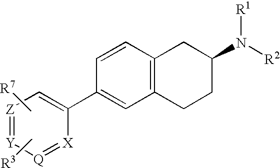

Another preferred embodiment includes compounds of formula i, selected from the group consisting of
3-(6-Pyrrolidin-1-yl-5,6,7,8-tetrahydro-naphthalen-2-yl)-benzamide;
Azetidin-1-yl-[4-(6-pyrrolidin-1-yl-5,6,7,8-tetrahydro-naphthalen-2-yl)-phenyl]-methanone;
N-Cyclobutyl-4-(6-pyrrolidin-1-yl-5,6,7,8-tetrahydro-naphthalen-2-yl)-benzamide;
N-Isobutyl-3-(6-pyrrolidin-1-yl-5,6,7,8-tetrahydro-naphthalen-2-yl)-benzamide;
4-(6-Pyrrolidin-1-yl-5,6,7,8-tetrahydronaphthalen-2-yl)-pyridine;
N-(2-Methoxy-ethyl)-3-(6-pyrrolidin-1-yl-5,6,7,8-tetrahydro-naphthalen-2-yl)-benzamide methanone;
N-(2-Hydroxy-ethyl)-N-methyl-3-(6-pyrrolidin-1-yl-5,6,7,8-tetrahydro-naphthalen-2-yl)-benzamide;
N-Cyclopropyl-3-(6-pyrrolidin-1-yl-5,6,7,8-tetrahydronaphthalen-2-yl)-benzamide;
3-(6-Pyrrolidin-1-yl-5,6,7,8-tetrahydro-naphthalen-2-yloxy)-benzamide;
5-(6-Pyrrolidin-1-yl-5,6,7,8-tetrahydronaphthalen-2-yl)-oxazole;
1-[6-(4-Methanesulfonyl-phenoxy)-1,2,3,4-tetrahydro-naphthalen-2-yl]-pyrrolidine;
N-(2-Hydroxy-ethyl)-N-methyl-4-(6-pyrrolidin-1-yl-5,6,7,8-tetrahydro-naphthalen-2-yl)-benzamide;
4-(6-Pyrrolidin-1-yl-5,6,7,8-tetrahydro-naphthalen-2-yl)-N-(tetrahydro-pyran-4-yl)-benzamide;
N-(2-Methoxy-ethyl)-4-(6-pyrrolidin-1-yl-5,6,7,8-tetrahydro-naphthalen-2-yl)-benzamide;
N-isobutyl-4-(6-pyrrolidin-1-yl-5,6,7,8-tetrahydro-naphthalen-2-yl)-benzamide;
1-[6-(4-Methoxy-phenoxy)-1,2,3,4-tetrahydro-naphthalen-2-yl]-pyrrolidine;
N,N-dimethyl-4-(6-pyrrolidin-1-yl-5,6,7,8-tetrahydro-naphthalen-2-yl)-benzamide;
Azetidin-1-yl-[4-(6-pyrrolidin-1-yl-5,6,7,8-tetrahydro-naphthalen-2-yl)-phenyl]-methanone;
N-Ethyl-N-methyl-4-(6-pyrrolidin-1-yl-5,6,7,8-tetrahydro-naphthalen-2-yl)-benzamide;
(+)-N-Ethyl-N-methyl-4-(6-pyrrolidin-1-yl-5,6,7,8-tetrahydro-naphthalen-2-yl)-benzamide;
(−)-N-Ethyl-N-methyl-4-(6-pyrrolidin-1-yl-5,6,7,8-tetrahydro-naphthalen-2-yl)-benzamide;
2-Methoxy-5-(6-pyrrolidin-1-yl-5,6,7,8-tetrahydro-naphthalen-2-yl)-pyridine;
3-(6-Pyrrolidin-1-yl-5,6,7,8-tetrahydro-naphthalen-2-yl)-pyridine;
2-Methoxy-3-(6-pyrrolidin-1-yl-5,6,7,8-tetrahydro-naphthalen-2-yl)-pyridine;
6-Methoxy-2-methyl-3-(6-pyrrolidin-1-yl-5,6,7,8-tetrahydro-naphthalen-2-yl)-pyridine;
N-Isopropyl-4-(6-pyrrolidin-1-yl-5,6,7,8-tetrahydro-naphthalen-2-yl)-benzamide;
N-Cyclobutyl-4-(6-pyrrolidin-1-yl-5,6,7,8-tetrahydro-naphthalen-2-yl)-benzamide;
4-(6-Pyrrolidin-1-yl-5,6,7,8-tetrahydro-naphthalen-2-yl)-phenol;
1-[6-(4-Methoxy-2,6-dimethyl-phenyl)-1,2,3,4-tetrahydro-naphthalen-2-yl]-pyrrolidine;
1-[6-(4-Methanesulfonyl-phenyl)-1,2,3,4-tetrahydro-naphthalen-2-yl]-pyrrolidine;
(R)-1-[6-(4-Methanesulfonyl-phenyl)-1,2,3,4-tetrahydro-naphthalen-2-yl]-pyrrolidine;
(S)-1-[6-(4-Methanesulfonyl-phenyl)-1,2,3,4-tetrahydro-naphthalen-2-yl]-pyrrolidine;
3-(6-Pyrrolidin-1-yl-5,6,7,8-tetrahydro-naphthalen-2-yl)-benzamide;
(S)-3-(6-Pyrrolidin-1-yl-5,6,7,8-tetrahydro-naphthalen-2-yl)-benzamide;
(R)-3-(6-Pyrrolidin-1-yl-5,6,7,8-tetrahydro-naphthalen-2-yl)-benzamide;
1-[6-(4-Methoxy-phenyl)-1,2,3,4-tetrahydro-naphthalen-2-yl]-pyrrolidine;
(R)-1-[6-(4-Methoxy-phenyl)-1,2,3,4-tetrahydro-naphthalen-2-yl]-pyrrolidine;
(S)-1-[6-(4-Methoxy-phenyl)-1,2,3,4-tetrahydro-naphthalen-2-yl]-pyrrolidine;
1-Isopropyl-4-[6-(4-methoxy-phenyl)-1,2,3,4-tetrahydro-naphthalen-2-yl]-piperazine;
(S)-(−)-1-[6-(4-Chloro-phenyl)-1,2,3,4-tetrahydro-naphthalen-2-yl]-pyrrolidine;
(R)-(+)-1-[6-(4-Chloro-phenyl)-1,2,3,4-tetrahydro-naphthalen-2-yl]-pyrrolidine;
3-Fluoro-1-[6-(4-methoxy-phenyl)-1,2,3,4-tetrahydro-naphthalen-2-yl]-pyrrolidine;
1-[4-(6-Pyrrolidin-1-yl-5,6,7,8-tetrahydro-naphthalen-2-yl)-phenyl]-ethanone;
3,4-Difluoro-1-[6-(4-methoxy-phenyl)-1,2,3,4-tetrahydro-naphthalen-2-yl]-pyrrolidine;
1-[6-(4-Methoxy-phenyl)-1,2,3,4-tetrahydro-naphthalen-2-yl]-2-methyl-pyrrolidine;
(R,R)-1-[6-(4-Methoxy-phenyl)-1,2,3,4-tetrahydro-naphthalen-2-yl]-2-methyl-pyrrolidine;
(S,R)-1-[6-(4-Methoxy-phenyl)-1,2,3,4-tetrahydro-naphthalen-2-yl]-2-methyl-pyrrolidine;
(R)-1-(6-bromo-1,2,3,4-tetrahydronaphthalen-2-yl)pyrrolidine;
(S)-1-(6-bromo-1,2,3,4-tetrahydronaphthalen-2-yl)pyrrolidine;
(R. R)-1-(6-Bromo-1,2,3,4-tetrahydro-naphthalen-2-yl)-2-methyl-pyrrolidine;
(S,R)-1-(6-Bromo-1,2,3,4-tetrahydro-naphthalen-2-yl)-2-methyl-pyrrolidine;
(R,S)-1-(6-Bromo-1,2,3,4-tetrahydro-naphthalen-2-yl)-2-methyl-pyrrolidine;
(S,S)-1-(6-Bromo-1,2,3,4-tetrahydro-naphthalen-2-yl)-2-methyl-pyrrolidine;
(R)-N,N-Dimethyl-3-(6-pyrrolidin-1-yl-5,6,7,8-tetrahydro-naphthalen-2-yl)-benzamide:
(R)-N,N-Dimethyl-3-(6-pyrrolidin-1-yl-5,6,7,8-tetrahydro-naphthalen-2-yl)-benzamide;
(S,R)-3-[6-(2-Methyl-pyrrolidin-1-yl)-5,6,7,8-tetrahydro-naphthalen-2-yl]-pyridine;
(R,R)-3-[6-(2-Methyl-pyrrolidin-1-yl)-5,6,7,8-tetrahydro-naphthalen-2-yl]-pyridine;
1-[6-(3,4-Dimethoxy-phenyl)-1,2,3,4-tetrahydro-naphthalen-2-yl]-2-methyl-pyrrolidine;
1-[6-(3-Fluoro-4-methoxy-phenyl)-1,2,3,4-tetrahydro-naphthalen-2-yl]-2-methyl-pyrrolidine;
N-Methyl-4-(6-pyrrolidin-1-yl-5,6,7,8-tetrahydro-naphthalen-2-yl)-benzamide;
4-[6-(2-Methyl-pyrrolidin-1-yl)-5,6,7,8-tetrahydro-naphthalen-2-yl]-benzamide;
3-[6-(2-Methyl-pyrrolidin-1-yl)-5,6,7,8-tetrahydro-naphthalen-2-yl]-benzamide;
(R,R)-3-[6-(2-Methyl-pyrrolidin-1-yl)-5,6,7,8-tetrahydro-naphthalen-2-yl]-benzamide;
(S,R)—[6-(2-Methyl-pyrrolidin-1-yl)-5,6,7,8-tetrahydro-naphthalen-2-yl]-benzamide;
1-[6-(4-Methanesulfonyl-phenyl)-1,2,3,4-tetrahydro-naphthalen-2-yl]-2-methyl-pyrrolidine;
   and pharmaceutically acceptable salts thereof.

This invention is also directed to pharmaceutical composition for treating a disorder or condition that may be treated by antagonizing histamine-3 receptors, the composition comprising a compound of formula I and optionally a pharmaceutically acceptable carrier.

This invention is also directed to a method of treatment of a disorder or condition that may be treated by antagonizing histamine-3 receptors, the method comprising administering to a mammal in need of such treatment a compound of formula I.

This invention is also directed to a method of treatment of a disorder or condition selected from the group consisting of depression, mood disorders, schizophrenia, anxiety disorders, cognitive disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I.

This invention is also directed to a pharmaceutical composition for treating allergic rhinitis, nasal congestion or allergic congestion comprising: (a) an H3 receptor antagonist compound of formula I or a pharmaceutically acceptable salt thereof; (b) an HI receptor antagonist or a pharmaceutically acceptable salt thereof; and (c) a pharmaceutically acceptable carrier; wherein the active ingredients (a) and (b) above are present in amounts that render the composition effective in treating allergy rhinitis, nasal congestion or allergic congestion.

This invention is also directed to a pharmaceutical composition for treating ADD, ADHD, depression, mood disorders, or cognitive disorders comprising: (a) an H3 receptor antagonist compound of Formula I or a pharmaceutically acceptable salt thereof; (b) a neurotransmitter re-uptake blocker or a pharmaceutically acceptable salt thereof; (c) a pharmaceutically acceptable carrier; wherein the active ingredients (a) and (b) above are present in amounts that render the composition effective in treating depression, mood disorders, and cognitive disorders.

In the general formula I according to the present invention, when a radical is mono- or poly-substituted, said substituent(s) can be located at any desired position(s), unless otherwise stated. Also, when a radical is polysubstituted, said substituents can be identical or different, unless otherwise stated.

The histamine-3 (H3) receptor antagonists of the invention are useful for treating, in particular, ADD, ADHD, obesity, anxiety disorders and respiratory diseases. Respiratory diseases that may be treated by the present invention include adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

The pharmaceutical composition and method of this invention may also be used for preventing a relapse in a disorder or condition described in the previous paragraphs. Preventing such relapse is accomplished by administering to a mammal in need of such prevention a compound of formula I as described above.

The disclosed compounds may also be used as part of a combination therapy, including their administration as separate entities or combined in a single delivery system, which employs an effective dose of a histamine H3 antagonist compound of general formula I and an effective dose of a histamine H1 antagonist, such as cetirizine (Zyrtec™), chlorpheniramine (Chlortrimeton™), loratidine (Claritin™), fexofenadine (Allegra™), or desloratadine (Clarinex™) for the treatment of allergic rhinitis, nasal congestion, and allergic congestion.

The disclosed compounds may also be used as part of a combination therapy, including their administration as separate entities or combined in a single delivery system, which employs an effective dose of a histamine H3 antagonist compound of general formula I and an effective dose of a neurotransmitter reuptake blocker. Examples of neurotransmitter reuptake blockers will include the serotonin-selective reuptake inhibitors (SSRI's) like sertraline (Zoloft™), fluoxetine (Prozac™), and paroxetine (Paxil™), or non-selective serotonin, dopamine or norepinephrine reuptake inhibitors for treating ADD, ADHD, depression, mood disorders, or cognitive disorders.

The compounds of the present invention may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, ¹⁵N, ¹⁸O, ¹⁷O, ¹⁵O, ³¹P, ³²P, ³⁵S, ¹⁸F, and ³⁶Cl, ¹²³I respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i., ²H, can afford certain therapeutic advantages resulting from greater metabolic stability, for example increased in vivo half-life or reduced dosage requirements and, hence, may be preferred in some circumstances.

Substitution with positron emitting isotopes, such as ¹¹C, ¹⁸F, ¹⁵O and ¹³N, can be useful in Positron Emission Topography (PET) studies for examining substrate receptor occupancy.

Anxiety disorders include, for example, generalized anxiety disorder, panic disorder, PTSD, and social anxiety disorder. Mood adjustment disorders include, for example, depressed mood, mixed anxiety and depressed mood, disturbance of conduct, and mixed disturbance of conduct and depressed mood. Attention adjustment disorders include, for example, in addition to ADHD, attention-deficit disorders or other cognitive disorders due to general medical conditions. Psychotic disorders include, for example, schizoaffective disorders and schizophrenia; sleep disorders include, for example, narcolepsy and enuresis.

Examples of the disorders or conditions which may be treated by the compound, composition and method of this invention are also as follows: depression, including, for example, depression in cancer patients, depression in Parkinson's patients, post-myocardial infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, diazepam, benzodiazepines and other psychoactive substances; Parkinson's diseases, including, for example, dementia in Parkinson's disease, neuroleptic-induced parkinsonism or tardive dyskinesias; headache, including, for example, headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

As an example, the mammal in need of the treatment or prevention may be a human. As another example, the mammal in need of the treatment or prevention may be a mammal other than a human.

Pharmaceutically acceptable salts of the compounds of formula I include the acid addition and base salts thereof.

Suitable acid addition salts are formed from acids that form non-toxic salts. Examples include the acetate, aspantate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts.

Suitable base salts are formed from bases that form non-toxic salts. Examples include the aluminium, arginine, benzathine, calcium, choline, diethylamine, diolamine, glycine, lysine, magnesium, meglumine, olamine, potassium, sodium, tromethamine and zinc salts.

Hemisalts of acids and bases may also be formed, for example, hemisulphate and hemicalcium salts.

For a review on suitable salts, see “Handbook of Pharmaceutical Salts: Properties, Selection, and Use” by Stahl and Wermuth (Wiley-VCH, Weinheim, Germany, 2002).

The compounds of the invention may exist in both unsolvated and solvated forms. The term ‘solvate’ is used herein to describe a molecular complex comprising the compound of the invention and a stoichiometric amount of one or more pharmaceutically acceptable solvent molecules, for example, ethanol. The term ‘hydrate’ is employed when said solvent is water.

Pharmaceutically acceptable solvates in accordance with the invention include those wherein the solvent of crystallization may be isotopically substituted, e.g. D₂O, d₆-acetone, d₆-DMSO.

Included within the scope of the invention are complexes such as clathrates, drug-host inclusion complexes wherein, in contrast to the aforementioned solvates, the drug and host are present in stoichiometric or non-stoichiometric amounts. Also included are complexes of the drug containing two or more organic and/or inorganic components, which may be in stoichiometric or non-stoichiometric amounts. The resulting complexes may be ionized, partially ionized, or non-ionized. For a review of such complexes, see J Pharm Sci, 64 (8), 1269-1288 by Haleblian (August 1975).

Hereinafter all references to compounds of formula I include references to salts, solvates and complexes thereof and to solvates and complexes of salts thereof.

The compounds of the invention include compounds of formula I as hereinbefore defined, including all polymorphs and crystal habits thereof, prodrugs and isomers thereof (including optical, geometric and tautomeric isomers) as hereinafter defined and isotopically-labeled compounds of formula I.

As indicated, so-called ‘pro-drugs’ of the compounds of formula I are also within the scope of the invention. Thus certain derivatives of compounds of formula I which may have little or no pharmacological activity themselves can, when administered into or onto the body, be converted into compounds of formula I having the desired activity, for example, by hydrolytic cleavage. Such derivatives are referred to as ‘prodrugs’. Further information on the use of prodrugs may be found in ‘Pro-drugs as Novel Delivery Systems, Vol. 14, ACS Symposium Series (T. Higuchi and W. Stella) and ‘Bioreversible Carriers in Drug Design’, Pergamon Press, 1987 (ed. E. B Roche, American Pharmaceutical Association).

Compounds of formula I containing one or more asymmetric carbon atoms can exist as two or more stereoisomers. Where structural isomers are interconvertible via a low energy barrier, tautomeric isomerism (‘tautomerism’) can occur. This can take the form of proton tautomerism in compounds of formula I containing, for example, an imino, keto, or oxime group, or so-called valence tautomerism in compounds that contain an aromatic moiety. It follows that a single compound may exhibit more than one type of isomerism.

Included within the scope of the present invention are all stereoisomers, geometric isomers and tautomeric forms of the compounds of formula I, including compounds exhibiting more than one type of isomerism, and mixtures of one or more thereof. Also included are acid addition or base salts wherein the counterion is optically active, for example, d-lactate or l-lysine, or racemic, for example, dl-tartrate or dl-arginine.

Unless otherwise indicated, the term “halo”, as used herein includes fluoro, chloro, bromo and iodo.

Unless otherwise indicated, the term ‘alkyl’, as used herein includes includes saturated monovalent hydrocarbon radicals having straight or branched moieties. Examples of alkyl groups include, but are not limited to, methyl, ethyl, propyl, isopropyl, and t-butyl.

Unless otherwise indicated, the term “alkoxy”, as used herein, includes straight-chain and branched alkoxy groups and includes for example methoxy, ethoxy, n-propoxy, i-propoxy, n-butoxy, i-butoxy, secbutoxy and t-butoxy.

Unless otherwise indicated, the term “alkylene”, as used herein, includes a divalent radical derived from straight-chain or branched alkane. Examples of alkylene radicals are methylene, ethylene (1,2-ethylene or 1,1-ethylene), trimethylene (1,3-propylene), tetramethylene (1,4-butylene), pentamethylene and hexamethylene.

Unless otherwise indicated, the term “cycloalkyl”, as used herein, unless otherwise indicated, includes non-aromatic saturated cyclic alkyl moieties wherein alkyl is as defined above. Examples of cycloalkyl include, but are not limited to, cyclopropyl, cyclobutyl, cyclopentyl, cyclohexyl, and cycloheptyl.

Unless otherwise indicated, the term “heterocycloalkyl”, as used herein, refer to non-aromatic cyclic groups containing one or more heteroatoms, preferably from one to four heteroatoms, each preferably selected from oxygen, sulfur and nitrogen. The heterocycloalkyl groups of this invention can also include ring systems substituted with one or more oxo moieties. Examples of non-aromatic heterocycloalkyl groups are aziridinyl, azetidinyl, pyrrolidinyl, piperidinyl, azepinyl, piperazinyl, 1,2,3,6-tetrahydropyridinyl, oxiranyl, oxetanyl, tetrahydrofuranyl, tetrahydrothienyl, tetrahydropyranyl, tetrahydrothiopyranyl, morpholino, thiomorpholino, thioxanyl, pyrrolinyl, indolinyl, 2H-pyranyl, 4H-pyranyl, dioxanyl, 1,3-dioxolanyl, pyrazolinyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, quinolizinyl, quinuclidinyl, 1,4-dioxaspiro[4.5]decyl, 1,4-dioxaspiro[4.4]nonyl, 1,4-dioxaspiro[4.3]octyl, and 1,4-dioxaspiro[4.2]heptyl.

Unless otherwise indicated, the term “aryl”, as used herein, includes and organic radical derived from an aromatic hydrocarbon by removal of one hydrogen, such as phenyl, napthyl, indenyl, and fluoroenyl. ‘Aryl’ encompasses fused ring groups wherein at least one ring is aromatic.

Unless otherwise indicated, the term “heteroaryl” as used herein, includes monocyclic or bicyclic heteroaryl groups having 5 to 9 and 9 to 14 ring members respectively, which contain 1, 2, 3 or 4 heteroatom(s) selected from nitrogen, oxygen and sulphur. The heteroaryl group can be unsubstituted, monosubstituted or disubstituted. Examples of heteroaryl groups include, but are not limited to thiophenyl, furanyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyranyl, pyridinyl, pyrazinyl, pyrimidinyl, pyridazinyl, triazinyl, thiadiazinyl, isobenzofuranyl, benzoturanyl, chromenyl, indolizinyl, isoindolyl, indolyl, indazolyl, purinyl, quinolinyl, isoquinolyl, cinnolinyl, phthalazinyl, naphthyridinyl, quinazolinyl, quinoxalinyl, benzoxazolyl, benzothiazolyl, benzimidazolyl, benzofuranyl, benzothiophenyl, pyrrolopyrazinyl, pyrrolopyridinyl, and imidazopyridinyl.

Unless otherwise indicated, the term “heterocyclic ring”, as used herein, refers to both heteroaryl and heterocycloalkyl groups, as defined above.

### DETAILED DESCRIPTION OF THE INVENTION

The compounds of the Formula I may be prepared by the methods described below, together with synthetic methods known in the art of organic chemistry, or modifications and derivatisations that are familiar to those of ordinary skill in the art. Preferred methods include, but are not limited to, those described below.

During any of the following synthetic sequences it may be necessary and/or desirable to protect sensitive or reactive groups on any of the molecules concerned. This can be achieved by means of conventional protecting groups, such as those described in T. W. Greene, Protective Groups in Organic Chemistry, John Wiley & Sons, 1981; and T. W. Greene and P. G. M. Wuts, Protective Groups in Organic Chemistry, John Wiley & Sons, 1991, which are hereby incorporated by reference.

Compounds of formula I, or their pharmaceutically acceptable salts, can be prepared according to the following reaction Schemes I through II as discussed herein below. Unless otherwise indicated X, Q, Y, Z and R₁ through R₅ are defined as above. Isolation and purification of the products is accomplished by standard procedures, which are known to a chemist of ordinary skill.

The following schemes are exemplary of the processes for making compounds of formula I.

Scheme I illustrates a method for the preparation of compounds having the basic structure of formula I, where R₁, R₂, R₃, Y, Q, Z and X are defined as above.

Referring to Scheme I, a compound of formula (II) can be prepared by treatment of a bromo-tetralone compound of formula (I) with an appropriately substituted amine reagent of formula (II) and a suitable reducing agent such as NaHB(OAc)₃ in a solvent such as CH₂Cl₂ or DCE, at temperatures ranging from −5° C. to room temperature, preferably at about room temperature, to produce the desired amine of formula (III). Other suitable reducing agents for this reaction include NaCNBH3 or NaBH₄, in solvents such as MeOH or EfOH. Other suitable conditions for this transformation include treatment of the corresponding tetralone of formula (I) with the amine reagent (II) in CH₂Cl₂ or DCE in the presence of 4 A molecular sieves and a base such as TEA at room temperature, followed by treatment with NaBH₄ or NaHB(OAc)₃. Compounds of formula (III) can then be treated with an appropriately substituted boronic acid of formula (IV), in the presence of a suitable palladium catalyst such as 1,1-bis(diphenylphosphino)ferrocene palladium (II) chloride and a suitable aqueous solution of an alkali base such as sodium carbonate and in solvents such as dimethoxy ethane, at temperatures ranging from room temperature to about 100° C., preferably at about 90° C., to produce the desired compound of formula (V). Other suitable conditions for this transformation include treatment of the compound of formula (III) and the appropriately substituted boronic acid of formula (IV) with tetrakis(triphenylphosphine)palladium(0) and sodium carbonate in ethanol/water mixture at temperatures ranging from 30° C. to 110° C., preferably at about the reflux temperature, to produce the corresponding compound of formula (V).

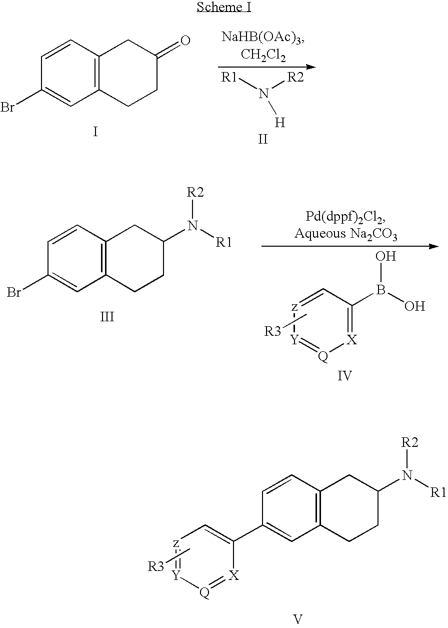

Scheme II illustrates an alternative method for the preparation of compounds having the basic structure of formula i, where R₅ is CONR₄R₅ and R₁, R₂, Y, Z, Q and X are defined as above. Coupling of the bromide (III) and a suitable boronic acid reagent of formula (VI) can be carried out as described above in scheme I to produce the desired compound of formula (VII). Treatment of the corresponding t-butyl ester derivative of formula (VII) with trifluoroacetic acid in methylene chloride at room temperature produces the corresponding carboxylic acid (not depicted). Treatment of the carboxylic acid with an amine of formula (VIII), in the presence of a suitable coupling reagent such as HOBT and EDCl, and a tertiary amine such as triethyl amine, can produce the desired compounds of formula (IX).

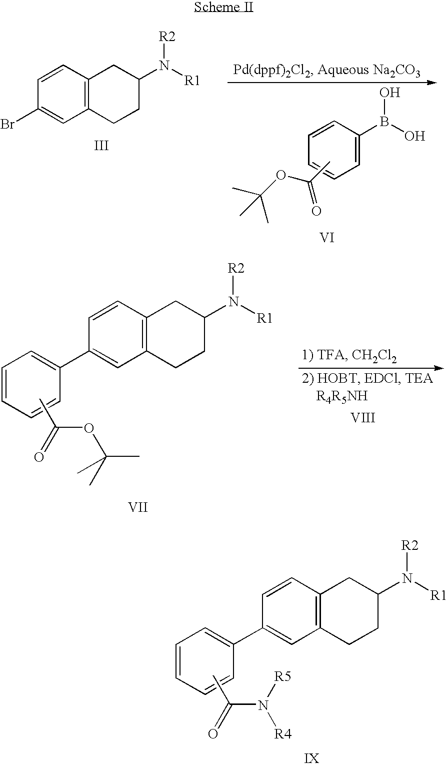

Alternatively, compounds of formula (IX) can also be prepared by treatment of the carboxylic acid and suitable amine of formula (VIII) with 2-chloro-1,3-dimethyl imidazoline chloride and a suitable base such as diisopropylethyl amine, in solvents such as methylene chloride.

The following examples and preparations illustrate the present invention. It is to be understood, however, that the invention, as fully described herein and as recited in the claims, TO is not intended to be limited by the details of the following examples.

### Preparation 1

### 1-(6-bromo-1,2,3,4-tetrahydronaphthalen-2-yl)pyrrolidine

To a solution of 10.0 g (43.84 mmol) 6-bromo-3,4-dihydronaphthalen-2-(1H)-one in 550 mL of methylene chloride in a round-bottomed flask was added dropwise 5.5 mL pyrrolidine (65.76 mmol) at room temperature. The solution became dark purple in color. After cooling the solution to 0° C., sodium triacetoxy borohydride (20.0 g, 87.68 mmol) was added in small portions. The reaction mixture was allowed to warm to room temperature and let stir overnight (15 hours). The reaction was then quenched with water (300 mL). Saturated sodium bicarbonate was added (200 mL) bringing the pH to 7. Solid sodium bicarbonate was added until the reaction became basic (pH 9). The organic layer was separated and washed with saturated sodium bicarbonate, water, then brine, and was dried (MgSO₄), filtered, and concentrated.

The crude product was then dissolved in ethyl acetate (200 mL). HCl in ethyl acetate was added and the reaction was stirred for a few minutes and then filtered to collect solid that crashed out. The solid product was washed with 50:50 hexanes/ethyl acetate and dried in vacuo to give 13.7 g, a 98.4% yield of 1-(6-bromo-1,2,3,4-tetrahydronaphthalen-2-yl)pyrrolidine. 400 MHz ¹H NMR (CD₃OD) δ 7.3 (s, 1H), 7.3 (d, J=8.29 Hz, 1H), 7.1 (d, J=8.29 Hz, 1H), 3.7 (m, 2H), 3.6 (m, 1H), 3.3 (m, 3H), 2.9-3.0 (m, 3H), 2.4 (m, 1H), 2.2 (m, 2H), 2.1 (m, 2H), 1.9 (m, 1H). MS (M+1) 280.3, 282.3. TLC (Silica Gel GF); R_{f}=0.50 in methylene chloride-methanol (4:1).

### Preparation 2

### Tert-butyl 3-(6-(pyrrolidin-1-yl)-5,6,7,8-tetrahydronaphthalen-2-yl)benzoate

To a solution of 1-(6-bromo-1,2,3,4-tetrahydronaphthalen-2-yl)pyrrolidine (9.0 g, 28.417 mmol) in dimethoxy ethane (165 mL) was added 3-t-butoxycarbonyl phenyl boronic acid (9.465 g, 42.6255 mmol). 2 M sodium carbonate solution (71 mL) and 1,1-bis(diphenylphosphino)ferrocene palladium (II) chloride (0.23 g, 0.248) were then added to the solution. The reaction was warmed to 90° C. and refluxed for 6 hours. LCMS and TLC analysis showed no starting material. The reaction was cooled to room temperature and concentrated. The reaction was diluted with ethyl acetate, washed with water ×3, brine, and was dried (MgSO₄), filtered, and concentrated in vacuo to give a crude yield of 13.45 g. The resulting solid was purified by flash column chromatography on 330 g silica gel, eluting with methylene chloride/methanol/NH₄OH (10:1:0.1). The pure fractions were collected and concentrated to yield 12.23 g Tert-butyl 3-(6-(pyrrolidin-1-yl)-5,6,7,8-tetrahydronaphthalen-2-yl)benzoate. 400 MHz ¹H NMR (CDCl₃) δ 8.2 (m, 1H), 7.9 (m, 1H), 7.7 (m, 1H), 7.4-7.5 (m, 1H), 7.3-7.4 (m, 2H) 7.1 (m, 1H), 2.7-3.1 (m, 6H), 2.5 (brs, 1H), 2.2 (m, 1H), 1.8 (m, 3H), 1.7-1.8 (m, 1H), 1.6 (s, 9H), 1.5 (m, 3H). MS (M+1) 378.3, 379.2.

### Preparation 3

### 3-(6-(Pyrrolidin-1-yl)-5,6,7,8-tetrahydronaphthalen-2-yl)benzoic Acid

Crude tert-butyl 3-(6-(pyrrolidin-1-yl)-5,6,7,8-tetrahydronaphthalen-2-yl)benzoate was diluted in methylene chloride Trifluoroacetic acid was added. The reaction stirred over night, LCMS showed no starting material, The reaction was concentrated to give a quantitative yield (14.0 g) of the desired TFA salt 3-(6-(pyrrolidin-1-yl)-5,6,7,8-tetrahydronaphthalen-2-yl)benzoic acid. 400 MHz ¹H NMR (CD₃OD) δ 8.2 (s, 1H), 8.0 (d, J=7.88 Hz, 1H), 7.8 (m, 1H), 7.5 (m, 1H), 7.4-7.5 (m, 2H), 7.3 (d, J=7.8 Hz, 1H), 3.8 (m, 2H), 3.6 (m, 1H), 3.3-3.4 (m, 1H), 3.2-3.3 (m, 2H), 3.0-3.1 (m, 3H), 2.4 (m, 1H), 2.2 (m, 2H), 2.0-2.1 (m, 2H), 1.9-2.0 (m, 1H). MS (M+1) 322.2, 323.2.

Other examples prepared according to the described procedure in preparation 3.

### 4-(6-Pyrrolidin-1-yl-5,6,7,8-tetrahydro-naphthalen-2-yl)-benzoic acid

400 MHz ¹H NMR (CD₃OD) δ 8.1 (d, J=8.3 Hz, 2H), 7.7 (d, J=8.7 Hz, 2H), 7.6 (m, 2H), 7.3 (d, J=8.7 Hz, 1H), 3.8 (m, 2H), 3.6 (m, 1H), 3.2-3.4 (m, 2H), 3.0-3.2 (m, 4H), 2.4 (m, 1H), 2.2 (m, 2H), 1.9-2.1 (m, 3H).

### General Procedure for Boronic Acid Coupling Reactions

To a solution of the bromo-amino-tetraline intermediate of formula (III) (1 equiv) in dimethoxy ethane (0.18 M) was added the boronic acid (1.5 equiv). 2 M sodium carbonate solution (5 equiv) and 1,1-bis(diphenylphosphino)ferrocene palladium (II) chloride (0.01 equiv) were then added to the solution. The reaction was warmed to 90° C. and refluxed for S6 hours. Reaction monitored by LCMS and TLC analysis. The reaction was cooled to room temperature and concentrated. The reaction was diluted with ethyl acetate, washed with water (×3), brine, and was dried (MgSO₄), filtered, and concentrated in vacuo to give the crude product. The resulting solid was purified by flash column chromatography eluting with methylene chloride/methanol/NH₄OH (10:1:0.1). The pure fractions were collected and concentrated to yield the desired product.

### Example 1

### N,N-dimethyl-3-(6-(pyrrolidin-1-Vi)-5,6,7,8-tetrahydronaphthalen-2-yl)benzamide

To a solution of 1-(6-bromo-1,2,3,4-tetrahydronaphthalen-2-yl)pyrrolidine (10.0 g, 31.575 mmol) in dimethoxy ethane (180 mL) was added the boronic acid (9.14, 47.36 mmol). 2 M sodium carbonate solution (78 mL, 157.88 mmol) and Pd(dppf)Cl₂ (0.316 g, 0.3157 mmol) were then added to the solution. The reaction was warmed to 100° C. and refluxed overnight (20 hours). LCMS and TLC analysis showed no starting material. The reaction was cooled to room temperature and concentrated. The reaction was diluted with ethyl acetate, washed with water (×3), brine, and was dried (MgSO₄), filtered, and concentrated in vacuo. The resulting solid was purified by flash column chromatography on 330 g silica gel, eluting with methylene chloride/methanol/NH₄OH (10:1:0.1). Fractions 30-65 gave 7.72 g pure N,N-dimethyl-3-(6-(pyrrolidin-1-yl)-5,6,7,8-tetrahydronaphthalen-2-yl)benzamide, a 70.3% yield. 400 MHz ¹H NMR (CDCl₃) δ 7.5-7.6 (m, 2H), 7.4 (m, 1H), 7.3 (m, 3H), 7.1 (d, J=7.9 Hz, 1H), 3.1 (s, 3H), 2.7-3.0 (m, 11H), 2.6 (m, 1H), 2.2 (m, 1H), 1.8-1.9 (m, 3H), 1.7-1.8 (m, 2H). MS (M+1) 349.3.

### Example 2

### N-Ethyl-N-methyl-3-(6-pyrrolidin-1-yl-5,6,7,8-tetrahydro-naphthalen-2-yl)-benzamide

To a solution of 3-(6-(pyrrolidin-1-yl)-5,6,7,8-tetrahydronaphthalen-2-yl)benzoic acid (prepared above, 1.5 g, 3.44 mmol) in 27.5 mL methylene chloride was added N-ethylmethyl amine (0.355 mL, 4.13 mmol) followed by HOBT (0.512 g, 3.79 mmol), EDCl (0.86 g, 4.478 mmol), and triethyl amine (2.4 mL, 17.224 mmol). The reaction stirred overnight at room temperature. The reaction was quenched with water and extracted (×3) with methylene chloride, dried (MgSO₄), filtered, and concentrated. The resulting solid was purified by flash column chromatography on 220 g silica gel, eluting with methylene chloride/methanol/NH₄OH (10:1:0.1). The pure fractions were collected and concentrated to give 775 mg N,N-dimethyl-3-(6-(pyrrolidin-1-yl)-5,6,7,8-tetrahydronaphthalen-2-yl)benzamide, a 62% yield. 400 MHz ¹H NMR (CDCl₃) δ 7.6 (m, 2H), 7.4 (m, 1H), 7.3 (m, 3H), 7.1 (d, J=7.8 Hz, 1H), 3.6 (m, 1H), 3.3 (m, 1H), 3.0-3.1 (s, 3H), 2.7-3.0 (m, 9H), 2.5 μm, 1H), 2.2 (m, 1H), 1.8-1.9 (m, 4H), 1.6-1.7 (m, 1H), 1.1-1.2 (m, 2H). MS (M+1) 363.3, 364.3. TLC (Silica Gel GF): R_{f}=0.25 in methylene chloride/methanol/NH₄OH (10:1:0.1).

### Alternative Amide Coupling Conditions for Preparation of N,N-dimethyl-3-(6-(pyrrolidin-1-yl)-5,6,7,8-tetrahydronaphthalen-2-yl)benzamide

To a solution of 3-(6-(pyrrolidin-1-yl)-5,6,7,8-tetrahydronaphthalen-2-yl)benzoic acid

(prepared above, 11.35 g, 26.06 mmol) in 130 mL of methylene chloride at a C (due to large scale of reaction) was added diisopropyl ethyl amine (22 mL, 130.3 mmol), followed by dimethyl amine HCL (3.19 g, 39.1 mmol). 2-chloro-1,3-dimethyl imidazoline chloride (4.4 g) was added to the reaction in portions, using methylene chloride to transfer the hydroscopic reagent. The reaction was removed from the ice bath and stirred at room temperature over night. (18 h). LCMS showed no starting material. The reaction was concentrated, then diluted with ethyl acetate and water. The organic layer was washed (3×) H₂O and brine, dried (MgSO₄), filtered, and concentrated to give 3.9 g crude N,N-dimethyl-3-(6-(pyrrolidin-1-yl)-5,6,7,8-tetrahydronaphthalen-2-yl)benzamide, 400 MHz ¹H NMR (CDCl₃) δ 7.5-7.6 (m, 2H), 7.4 (m, 1H), 7.3 (m, 3H), 7.1 (d, J=7.9 Hz, 1H), 3.1 (s, 3H), 2.7-3.0 (m, 11H), 2.6 (m, 1H), 2.2 (m, 1H), 1.8-1.9 (m, 3H), 1.7-1.8 (m, 2H); 349.3.

### General Salt Formation:

The free base was dissolved in ethyl acetate. Saturated HCl in ethyl acetate was added to the solution and allowed to stir for five minutes, then concentrated in vacuo giving the resulting HCL salt.

The following examples were prepared utilizing the procedures and examples described above.

### Example 3

### 3-(6-Pyrrolidin-1-yl-5,6,7,8-tetrahydro-naphthalen-2-yl)-benzamide

400 MHz ¹H NMR (CD₃OD) δ 8.1 (t, J=1.7 Hz, 1H), 7.8 μm, 2H), 7.5 (d, J=7.9 Hz, 1H), 7.4 (m, 2H), 7.2-7.3 (d, J=7.9 Hz, 1H), 3.7-3.8 (m, 2H), 3.6 (m, 1H), 3.3-3.4 (m, 1H), 3.2-3.3 (m, 2H), 3.0-3.1 (m, 3H), 2.4 (m, 1H), 2.2 (m, 2H), 1.9-2.1 (m, 3H).

### Example 4

### N-Isopropyl-4-(6-pyrrolidin-1-yl-5,6,7,8-tetrahydro-naphthalen-2-yl)-benzamide

400 MHz ¹H NMR (CD₃OD) δ 7.9 (t, J=1.7 Hz, 1H), 7.6-7.7 (m, 2H), 7.4-7.5 (t, J=7.6 Hz, 1H), 7.3-7.4 (m, 2H), 7.1 (d, J=7.9 Hz, 1H), 5.9-6.0 (m, 1H), 4.2-4.3 (m, 1H), 3.0-3.1 (m, 1H), 2.8-3.0 (m, 3H), 2.7-2.8 (m, 4H), 2.5 (brs, 1H), 2.2 (m, 1H), 1.8 (m, 4H), 1.7 (m, 1H), 1.2-1.3 (d, J=6.6 Hz, 6H).

### Example 5

### Azetidin-1-yl-[4-(6-pyrrolidin-yl-5,6,7,8-tetrahydro-naphthalen-2-yl)-phenyl]-methanone

400 MHz ¹H NMR (CD₃OD) δ 7.8 (t, J=1.7 Hz, 1H), 7.6 (m, 1H), 7.5 (m, 1H), 7.4 (t, J=7.6 Hz, 1H), 7.3 (m, 2H), 7.1 (d, J=7.9 Hz, 1H), 4.3 (t, J=7.6 Hz, 2H), 4.2 (t, J=7.6 Hz, 2H), 3.0-3.1 (m, 1H), 2.8-3.0 (m, 3H), 2.7 (m, 4H), 2.4-2.5 (m, 1H), 2.3-2.4 (m, 2H), 2.2-2.3 (m, 1H), 1.8-1.9 (m, 4H), 1.7 (m, 1H).

### Example 6

### N-Cyclobutyl-4-(6-pyrrolidin-1-yl-5,6,7,8-tetrahydro-naphthalen-2-yl)-benzamide

400 MHz ¹H NMR (CD₃OD) δ 7.9 (t, J=1.7 Hz, 1H), 7.6-7.7 (m, 2H), 7.4-7.5 (t, J=7.4 Hz, 1H), 7.3 (m, 2H), 7.1-7.2 (d, J=7.5 Hz, 1H), 6.3 (d, J=7.5 Hz, 1H), 4.6 (m, 1H), 2.8-3.1 (m, 4H), 2.7 (m, 4H), 2.4-2.5 (m, 3H), 2.2 (m, 1H), 1.7-2.0 (m, 8H).

### Example 7

### N-Isobutyl-4-(6-pyrrolidin-1-yl-5,6,7,8-tetrahydro-naphthalen-2-yl)-benzamide

400 MHz ¹H NMR (CD₃OD) δ 7.9 (t, J=1.7 Hz, 1H), 7.6-7.7 (m, 2H), 7.4-7.5 (t, J=7.9 Hz, 1H), 7.3-7.4 (m, 2H), 7.1-7.2 (d, J=7.5 Hz, 1H), 6.1 (m, 1H), 3.3 (m, 2H), 3.0-3.1 (dd, J=16.2, 3.7 Hz, 1H), 2.8-3.0 (m, 7H), 2.6 (brs, 1H), 2.2-2.3 (m, 1H), 1.8-1.9 (m, 6H), 1.2-1.3 (d, J=6.6 Hz, 6H).

### Example 8

### 4-(6-Pyrrolidin-1-yl-5,6,7,8-tetrahydro-naphthalen-2-yl)-pyridine

400 MHz H NMR (CDCl₃) δ 8.6 (m, 2H), 7.4-7.5 (m, 2H), 7.3-7.4 (m, 2H), 7.2 (d, J=7.9 Hz, 1H), 2.8-3.1 (m, 4H), 2.7 (m, 4H), 2.4-2.5 (m, 1H), 2.2 (m, 1H), 1.7-1.9 (m, 5H).

### Example 9

### N-(2-Methoxy-ethyl)-3-(6-pyrrolidin-1-yl-5,6,7,8-tetrahydro-naphthalen-2-yl)-benzamide

400 MHz ¹H NMR (CDCl₃) 8.0 (t, J=1.7 Hz, 1H), 7.6-7.7 (m, 2H), 7.4-7.5 (t, J=7.6 Hz, 1H), 7.3-7.4 (m, 2H), 7.1-7.2 (d, J=7.9 Hz, 1H), 6.6 (brs, 1H), 3.6-3.7 (m, 2H), 3.5-3.6 (m, 2H), 3.4 (s, 3H), 2.8-3.2 (m, 4H), 2.7-2.8 (m, 4H), 2.5 (m, 1H), 2.2 (m, 1H), 1.8 (m, 4H), 1.6-1.8 (m, 1H).

### Example 10

### (3-Fluoro-azetidin-1-yl)-[3-(6-pyrrolidin-1-yl-5,6,7,8-tetrahydro-naphthalen-2-yl)-phenyl]-methanone

400 MHz ¹H NMR (CDCl₃) δ 7.8 (t, J=1.4 Hz, 1H), 7.6-7.7 (m, 1H), 7.5 (m, 1H), 7.4-7.5 (t, J=7.7 Hz, 1H), 7.3 (m, 2H), 7.1-7.2 (d, J=7.9 Hz, 1H), 5.2-5.4 (m, 1H), 4.2-4.6 (m, 4H), 3.1 (m, 1H), 2.9-3.0 (m, 3H), 2.7-2.9 (m, 4H), 2.5 (m, 1H), 2.2 (m, 1H), 1.8 (m, 4H), 1.7-1.8 (m, 1H).

### Example 11

### N-(2-Hydroxy-ethyl)-N-methyl-3-(6-pyrrolidin-1-VI-5,6,7,8-tetrahydro-naphthalen-2-yl)-benzamide

400 MHz ¹H NMR (CDCl₃) δ 7.6 (m, 2H), 7.3-7.5 (m, 4H), 7.1-7.2 (d, J=7.9 Hz, 1H), 3.9 (brs, 1H), 3.7 (m, 2H), 3.3-3.5 (m, 1H), 2.7-3.1 (m, 10H), 2.5 (brs, 1H), 2.2 (m, 1H), 1.6-1.8 (m, 7H).

### Example 12

### N-Cyclopropyl-3-(6-pyrrolidin-1-yl-5,6,7,8-tetrahydro-naphthalen-2-yl)-benzamide

400 MHz ¹H NMR (CDCl₃) δ 7.9 (t, J=1.7 Hz, 1H), 7.6-7.7 (m, 2H), 7.4-7.5 (t, J=7.7 Hz, 1H), 7.3 (m, 2H), 7.1-7.2 (d, J=7.9 Hz, 1H), 6.3 (brs, 1H), 3.0-3.1 (m, 1H), 2.8-3.0 (m, 4H), 2.7-2.8 (m, 4H), 2.5 μm, 1H), 2.2-2.3 (m, 1H), 1.8-1.9 (m, 4H), 1.7-1.8 (m, 1H), 0.8-1.9 (m, 2H), 0.6-0.7 (m, 2H).

### Example 13

### 3-(6-Pyrrolidin-1-yl-5,6,7,8-tetrahydro-naphthalen-2-yloxy)-benzamide

400 MHz ¹H NMR (CDCl₃) δ 7.4-7.5 (m, 1H), 7.3-7.4 (m, 2H), 7.1 (m, 1H), 7.0 (d, J=8.3 Hz, 1H), 6.7-6.8 (2H), 6.1 (brs, 1H), 5.8 (brs, 1H), 2.7-3.0 (m, 8H), 2.4-2.5 (m, 1H), 2.1-2.2 (m, 1H), 1.8 (m, 4H), 1.6-1.7 (m, 1H).

### Example 14

### 5-(6-Pyrrolidin-1-yl-5,6,7,8-tetrahydro-naphthalen-2-yl)-oxazole

400 MHz ¹H NMR (CDCl₃) δ 7.9 (s, 1H), 7.4 (d, J=7.9 Hz, 2H), 7.3 (s, 1H), 7.0-7.1 (m, 1H), 2.7-3.1 (m, 4H), 2.6-2.7 (m, 4H), 2.4 (m, 1H), 2.2 (m, 1H), 1.8 (m, 4H), 1.6-1.7 (m, 1H).

### Example 15

### 1-[6-(4-Methanesulfonyl-phenoxy)-1,2,3,4-tetrahydro-naphthalen-2-yl]-pyrrolidine

400 MHz ¹H NMR (CDCl₃) δ 7.8-7.9 (m, 2H), 7.1 (d, J=8.2 Hz, 1H), 7.0-7.1 (m, 2H), 6.7-6.8 (m, 2H), 3.0 (s, 3H), 2.7-3.0 (m, 8H), 2.4-2.5 (m, 1H), 2.2 (m, 1H), 1.8-1:9 (m, 4H), 1.6-1.7 (m, 1H).

### Example 16

### N-(2-Hydroxy-ethyl)-N-methyl-4-(6-pyrrolidin-1-yl-5,6,7,8-tetrahydro-naphthalen-2-yl)-benzamide

400 MHz ¹H NMR (CDCl₃) δ 7.6 (m, 2H), 7.5 (m, 2H), 7.3 (m, 2H), 7.1-7.2 (d, J=7.9 Hz, 1H), 3.9 (brs, 1H), 3.7 (m, 2H), 3.4-3.5 (m, 1H), 3.0-3.1 (m, 3H), 2.8-3.0 (m, 8H), 2.6 (brs, 1H), 2.2-2.3 (m, 1H), 1.8-1.9 (m, 6H).

### Example 17

### 4-(6-Pyrrolidin-1-yl-5,6,7,8-tetrahydro-naphthalen-2-yl)-N-(tetrahydro-pyran-4-yl)-benzamide

400 MHz ¹H NMR (CDCl₃) 7.8 (m, 2H), 7.6 (m, 2H), 7.3-7.4 (m, 2H), 7.2 (d, J=7.9 Hz, 1H), 6.0 (d, J=7.5 Hz, 1H), 4.2 (m, 1H), 4.0 (m, 2H), 3.5-3.6 (m, 2H), 3.03 (dd, J=16.6, 4.2 Hz, 1H), 2.8-3.0 (m, 3H), 2.7 (m, 4H), 2.5 (m, 1H), 2.2 (m, 1H), 20 (dd, J=12.4, 2.5 Hz, 2H), 1.5-1.8 (m, 7H).

### Example 18

### N-(2-Methoxy-ethyl)-4-(6-pyrrolidin-1-yl-5,6,7,8-tetrahydro-naphthalen-2-yl)-benzamide

400 MHz ¹H NMR (CDCl₃) δ 7.8 (d, J=8.3 Hz, 2H), 7.6 (d, J=8.3 Hz, 2H), 7.4 (d, J=7.9 Hz, 1H), 7.3 (s, 1H), 7.1-7.2 (d, J=7.9 Hz, 1H), 6.6 (m, 1H), 3.6-3.7 (m, 2H), 3.5-3.6 (m, 2H), 3.4 (s, 3H), 2.9-3.4 (m, 8H), 2.4 (m, 1H), 2.1-2.2 (m, 5H), 1.8-1.9 (m, 1H).

### Example 19

### N-Isobutyl-4-(6-pyrrolidin-1-yl-5,6,7,8-tetrahydro-naphthalen-2-yl)-benzamide

400 MHz ¹H NMR (CDCl₃) δ 7.8 (m, 2H), 7.6 (m, 2H), 7.3-7.4 (m, 2H), 7.2 (d, J=7.9 Hz, 1H), 6.6 (t, J=5.8 Hz, 1H), 3.3 (t, J=7.9 Hz, 2H), 3.0-3.1 (m, 1H), 2.8-3.0 (m, 3H), 2.7 (m, 4H), 2.4-2.5 (m, 1H), 2.1-2.3 (m, 1H), 1.6-1.9 (m, 6H), 1.0 (d, J=6.6 Hz, 6H).

### Example 20

### 1-[6-(4-Methoxy-phenoxy)-1,2,3,4-tetrahydro-naphthalen-2-yl]-pyrrolidine

400 MHz ¹H NMR (CDCl₃) S 7.0 (m, 3H), 6.8-6.9 (m, 2H), 6.7 (m, 1H), 6.6 (m, 1H), 3.8 (s, 3H), 3.0 (m, 1H), 2.7-2.8 (m, 7H), 2.4-2.5 (m, 1H), 2.1-2.2 (m, 1H), 1.8-1.9 (m, 4H), 1.6-1.7 (m, 1H).

### Example 21

### N,N-dimethyl-4-(6-pyrrolidin-1-yl-5,6,7,8-tetrahydro-naphthalen-2-yl)-benzamide

400 MHz ¹H NMR (CDCl₃) δ 7.6 (m, 2H), 7.4-7.5 (m, 2H), 7.31 (m, 2H), 7.1-7.2 (d, J=7.5 Hz, 1H), 2.7-3.1 (m, 12H), 2.4 (m, 1H), 2.2 (m, 1H), 1.6-1.8 (m, 7H).

### Example 22

### Azetidin-1-yl-[4-(6-Pyrrolidin-1-yl-5,6,7,8-tetrahydro-naphthalen-2-yl)-phenyl]-methanone

400 MHz ¹H NMR (CDCl₃) δ 7.6-7.7 (m, 2H), 7.6 (m, 2H), 7.3 (m, 2H), 7.1-7.2 (d, J=7.9 Hz 1H), 4.3-4.4 (d, J=7.7 Hz, 2H), 4.2 (d, J=7.7 Hz, 2H), 3.0-3.1 (m, 1H), 2.8-3.0 (m, 4H), 2.7 (m, 4H), 2.4-2.5 (m, 1H), 2.2-2.4 (m, 2H), 2.2 (m, 1H), 1.7-1.8 (m, 5H).

### Example 23

### N-Ethyl-N-methyl-4-(6-pyrrolidin-1-yl-5,6,7,8-tetrahydro-naphthalen-2-yl)-benzamide

400 MHz ¹H NMR (CDCl₃) δ 7.5-7.6 (m, 2H), 7.4 μm, 2H), 7.3 (m, 2H), 7.1-7.2 (d, J=7.9 Hz, 1H), 3.6 (m, 1H), 3.3 (m, 1H), 2.6-3.1 (m, 10H), 2.4 (m, 1H), 2.2 (m, 1H), 1.8 (m, 4H), 1.6-1.7 (m, 2H), 1.1-1.3 (m, 3H).

### Example 24

### (+)-N-Ethyl-N-methyl-4-(6-pyrrolidin-1-yl-5,6,7,8-tetrahydro-naphthalen-2-yl)-benzamide

400 MHz ¹H NMR (CD₃OD) δ 7.7 (d, J=7.9 Hz, 1H), 7.6 (d, J=7.5 Hz, 1H), 7.5 (t, J=7.8 Hz, 1H), 7.4 (m, 2H), 7.3 (m, 1H), 7.2-7.3 (d, J=7.9 Hz, 1H), 3.7-3.8 (m, 2H), 3.6 (m, 2H), 3.2-3.3 (m, 3H), 3.0-3.1 (m, 6H), 2.4 (m, 1H), 2.2 (m, 2H), 1.9-2.0 (m, 3H), 1.2-1.3 (t, J=7.1 Hz, 1H), 1.1-1.2 (m, 3H). MS (M+1) 363.4. [_]=(+) 34.32. Chiralcel OJ, Mobile Phase 9515 Heptane/EtOH, T_{R}=15.162 min.

### Example 24

### (−)-N-Ethyl-N-methyl-4-(6-pyrrolidin-1-yl-5,6,7,8-tetrahydro-naphthalen-2-yl)-benzamide

400 MHz ¹H NMR (CD₃OD) δ 7.7 (d, J=7.9 Hz, 1H), 7.6 (d, J=7.5 Hz, 1H), 7.5 (t, J=7.8 Hz, 1H), 7.4 (m, 2H), 7.3 (m, 1H), 7.2-7.3 (d, J=7.9 Hz, 1H), 3.7-3.8 (m, 2H), 3.6 (m, 2H), 3.2-3.3 (m, 3H), 3.0-3.1 (m, 6H), 2.4 (m, 1H), 2.2 (m, 2H), 1.9-2.0 (m, 3H), 1.2-1.3 (m, 2H), 1.1-1.2 (m, 2H). MS (M+1) 363.4. [_]=(−) 31.56. Chiralcel OJ, Mobile Phase 95/5 Heptane/EtOH, T_{R}=18.131 min.

### Example 25

### 2-Methoxy-5-(6-pyrrolidin-1-yl-5,6,7,8-tetrahydro-naphthalen-2-yl)-pyridine

400 MHz ¹H NMR (CDCl₃) δ 8.3 (dd, J=2.5, 0.8 Hz, 1H), 7.7-7.8 (dd, J=8.3, 2.5 Hz, 1H), 7.2-7.3 (m, 2H), 7.2 (d, J=7.9 Hz, 1H), 6.8 (m, 1H), 4.0 (s, 3H), 3.0-3.1 (m, 1H), 2.8-3.0 (m, 3H), 2.7 (m, 4H), 2.5 (m, 1H), 2.2-2.3 (m, 1H), 1.8-1.9 (m, 4H), 1.7-1.8 (m, 1H).

### Example 26

### 3-(6-Pyrrolidin-1-yl-5,6,7,8-tetrahydro-naphthalen-2-yl)-pyridine

400 MHz ¹H NMR (CDCl₃) δ 8.8 (d, J=1.7 Hz, 1H), 8.5 (d, J=4.5 Hz, 1H), 7.8 (m, 1H), 7.3 (m, 3H), 7.2 (d, J=7.9 Hz, 1H), 3.0-3.1 (m, 1H), 2.7-3.0 (m, 7H), 2.5 (m, 1H), 2.2-2.3 (m, 1H), 1.7-1.9 (m, 5H).

### Example 27

### 2-Methoxy-3-(6-pyrrolidin-1-yl-5,6,7,8-tetrahydro-naphthalen-2-yl)-pyridine

400 MHz ¹H NMR (CDCl₃) δ 8.1 (m, 1H), 7.5-7.6 (m, 1H), 7.3 (m, 1H), 7.2 (d, J=1.7 Hz, 1H), 7.1 (d, J=7.9 Hz, 1H), 7.0 (m, 1H), 4.0 (s, 3H), 3.0-3.1 (m, 1H), 2.8-3.0 (m, 3H), 2.7 (m, 4H), 2.5 (m, 1H), 2.2 (m, 1H), 1.8-1.9 (m, 4H), 1.7-1.8 (m, 1H).

### Example 28

### 6-Methoxy-2-methyl-3-(6-pyrrolidin-1-yl-5,6,7,8-tetrahydro-naphthalen-2-yl)-pyridine

400 MHz ¹H NMR (CDCl₃) δ 7.4 (d, J=8.3 Hz, 1H), 7.1 (d, J=7.7 Hz, 1H), 7.0 (m, 2H), 6.6 (d, J=7.9 Hz, 1H), 3.9 (s, 3H), 3.1 (m, 1H), 2.8-3.0 (m, 3H), 2.7 (m, 4H), 2.5 (m, 1H), 2.4 (s, 3H), 2.2 (m, 1H), 1.8-1.9 (m, 4H), 1.7 (m, 1H).

### Example 29

### N-Isopropyl-4-(6-pyrrolidin-1-yl-5,6,7,8-tetrahydro-naphthalen-2-yl)-benzamide

400 MHz ¹H NMR (CDCl₃) δ 7.8 (d, J=8.3 Hz, 2H), 7.6 (d, J=8.3 Hz, 2H), 7.3 (m, 2H), 7.2 (d, J=7.9 Hz, 1H), 6.0 (d, J=7.5 Hz, 1H), 4.3 (m, 1H), 3.1 (dd, J=15.8, 4.1 Hz, 1H), 2.8-3.0 (m, 3H), 2.7 (m, 4H), 2.5 (m, 1H), 2.2 (m, 1H), 1.8 (m, 4H), 1.6-1.8 (m, 1H), 1.3 (d, J=6.6 Hz, 6H).

### Example 30

### N-Cyclobutyl-4-(6-pyrrolidin-1-yl-5,6,7,8-tetrahydro-naphthalen-2-yl)-benzamide

400 MHz ¹H NMR (CDCl₃) δ 7.8 (m, 2H), 7.6 (m, 2H), 7.3-7.4 (m, 2H), 7.2 (d, J=7.5 Hz, 1H), 6.2 (d, J=7.9 Hz, 1H), 4.6 (m, 1H), 3.0-3.1 (m, 1H), 2.8-3.0 (m, 3H), 2.7 (m, 4H), 2.4-2.5 (m, 2H), 2.2 (m, 1H), 1.6-2.0 (m, 10H).

### Example 31

### 4-(6-Pyrrolidin-1-yl-5,6,7,8-tetrahydro-naphthalen-2-yl)-phenol

400 MHz ¹H NMR (DMSO) δ 7.4 (m, 2H), 7.2 (m, 2H), (7.0 (m, 1H), 6.8 (m, 2H), 2.5-2.9 (m, 8H), 2.3 brs, 1H), 2.0 (brs, 1H), 1.5-1.6 (m, 5H).

### Example 32

### 1-[6-(4-Methoxy-2,6-dimethyl-phenyl)-1,2,3,4-tetrahydro-naphthalen-2-yl]-pyrrolidine

400 MHz ¹H NMR (CD₃OD) δ 7.2 (d, J=7.5 Hz, 1H), 6.9 (m, 2H), 6.6 (s, 2H), 3.7-3.8 (m, 5H), 3.6 (m, 1H), 3.4 (m, 1H), 3.0-3.1 (m, 4H), 2.4 (m, 1H), 2.2 (m, 2H), 2.1 (m, 2H), 1.9-2.0 (m, 8H). MS (M+1) 336.4.

### Example 33

### 1-[6-(4-Methanesulfonyl-phenyl)-1,2,3,4-tetrahydro-naphthalen-2-yl]-pyrrolidine

400 MHz ¹H NMR (CDCl₃) δ 8.0 (d, J=8.3 Hz, 2H), 7.7 (d, J=8.3 Hz, 2H), 7.3-7.4 (m, 2H), 7.2 (d, J=7.9 Hz, 1H), 3.1-3.2 (m, 1H), 3.1 (s, 3H), 2.8-3.0 (m, 3H), 2.8 (m, 4H), 2.5 (m, 1H), 2.2-2.3 (m, 1H), 1.7-1.9 (m, 5H). MS-(M+1) 356.3.

### Example 34

### (R)-1-[6-(4-Methanesulfonyl-phenyl)-1,2,3,4-tetrahydro-naphthalen-2-yl]-pyrrolidine

400 MHz ¹H NMR (CDCl₃) δ 8.0 (m, 2H), 7.7 (m, 2H), 7.3-7.4 (m, 2H), 7.2 (d, J=7.9 Hz, 1H), 3.1-3.2 (m, 1H), 3.1 (s, 3H), 2.8-3.0 (m, 3H), 2.8 (m, 4H), 2.6 (m, 1H), 2.2-2.3 (m, 1H), 1.7-1.9 (m, 5H); MS (M+1) 356.2. Chiralcel OJ, Mobile Phase 30170 Heptane/EtOH, T_{R}=9.431 min.

### Example 35

### (S)-1-[6-(4-Methanesulfonyl-phenyl)-1,2,3,4-tetrahydro-naphthalen-2-yl]-pyrrolidine

400 MHz ¹H NMR (CD₃OD) δ 8.0 (d, J=8.3 Hz, 1H), 7.8-7.9 (d, J=8.3 Hz, 1H), 7.5 (t, J=7.1 Hz, 1H), 7.3 (m, 3H), 7.1 (d, J=8.3 Hz, 1H), 3.3-3.8 (m, 5H), 2.9-3.2 (m, 7H), 2.4 (m, 1H), 1.8-2.2 (m, 5H); MS (M+1) 356.4. Chiralcel OJ, Mobile Phase 30170 Heptane/EtOH, T_{R}=13.911 min.

### Example 36

### 3-(6-Pyrrolidin-1-yl-5,6,7,8-tetrahydro-naphthalen-2-yl)-benzamide

400 MHz ¹H NMR (CDCl₃) δ 8.1 (s, 1H), 7.7-7.8 (m, 2H), 7.5 (m, 1H), 7.4 (m, 2H), 7.2 (d, J=8-3 Hz, 1H), 3.1 (m, 1H), 2.7-2.9 (m, 7H), 2.5 (m, 1H), 2.2 (m, 1H), 1.8 (m, 4H), 1.6-1.7 (m, 1H); MS (M+1) 321.3, 322.4.

### Example 37

### (S)-3-(6-Pyrrolidin-1-yl-5,6,7,8-tetrahydro-naphthalen-2-yl)-benzamide

Chiralpak AS, Mobile Phase 80/20 Heptane/EtOH T_{R}=14.006 min. MS (M+1) 321.4.

### Example 38

### (R)-3-(6-Pyrrolidin-1-yl-5,6,7,8-tetrahydro-naphthalen-2-yl)-benzamide

Chiralpak AS, Mobile Phase 80/20 Heptane/EtOH T_{R}=9.253 min, MS (M+1) 321.4.

### Example 39

### 1-[6-(4-Methoxy-phenyl)-1,2,3,4-tetrahydro-naphthalen-2-yl]-pyrrolidine

400 MHz ¹H NMR (CDCl₃) δ 7.5 (m, 2H), 7.2-7.3 (m, 2H), 7.1 (d, J=7.9 Hz, 1H), 6.9-7.0 (m, 2H), 3.8 (s, 3H), 3.0-3.1 (dd, J=16.2, 3.7 Hz, 1H), 2.8-3.0 (m, 3H), 2.7 (m, 4H), 2.4 (m, 1H), 2.2 (m, 1H), 1.8 (m, 4H), 1.6-1.7 (m, 1H). MS (M+1) 308.2.

### Example 40

### (R)-1-[6-(4-Methoxy-phenyl)-1,2,3,4-tetrahydro-naphthalen-2-yl]-pyrrolidine

Chiralpak AS, Mobile Phase 80/20 Heptane/EtOH T_{R}=10.248 min. MS (M+1) 308.3.

### Example 41

### (S)-1-[6-(4-Methoxy-phenyl)-1,2,3,4-tetrahydro-naphthalen-2-yl]-pyrrolidine

Chiralpak AS, Mobile Phase 80/20 Heptane/EtOH T_{R}=12.360 min. MS (M+1) 308.2.

### Example 42

### 1-Isopropyl-4-[6-(4-methoxy-phenyl)-1,2,3,4-tetrahydro-naphthalen-2-yl]-piperazine

400 MHz ¹H NMR (CDCl₃) δ 7.5 (m, 2H), 7.3 (d, J=7.9 Hz, 2H), 7.1 (d, J=7.9 Hz, 1H), 7.0 (m, 2H), 3.8 (s, 3H), 2.6-3.0 (m, 13H), 2.2 (m, 1H); 1.6 (m, 2H), 1.0 (d, J=6.2 Hz 6H). MS (M+1) 365.3.

### Example 43

### (S)-(−)-1-[6-(4-Chloro-phenyl)-1,2,3,4-tetrahydro-naphthalen-2-yl]-pyrrolidine

400 MHz ¹H NMR (CD₃OD) δ 7.6 (d, J=8.3 Hz, 2H), 7.4 (d, J=8.7 Hz, 4H), 7.2 (d, J=7.5 Hz, 1H), 3.8 (m, 2H), 3.6 (m, 1H), 3.2-3.3 (m, 3H), 3.0-3.1 (m, 4H), 2.4 (m, 1H), 2.2 (m, 2H), 2.0 (m, 2H); MS (M+1) 312.3, 313.4. [_]=(−) 41.39. Chiralpak OJ, Mobile Phase 90110 Heptane/IPO, T_{R}=6.488 min.

### Example 44

### (R)-(+)-1-[6-(4-Chloro-phenyl)-1,2,3,4-tetrahydro-naphthalen-2-yl]-pyrrolidine

400 MHz ¹H NMR (CD₃OD) δ 7.6 (m, 2H), 7.3-7.4 (m, 4H), 7.2 (d, J=7.9 Hz, 1H), 3.8 (m, 2H), 3.6 (m, 1H), 3.2-3.3 (m, 3H), 3.0-3.1 (m, 4H), 2.4 (m, 1H), 2.2 (m, 2H), 2.0 (m, 2H): MS (M+1) 312.3, 313.4, [_]=(−) 39.58. Chiralpak OJ, Mobile Phase 90110 Heptane/IPO, T_{R}=5.573 min.

### Example 45

### 3-Fluoro-1-[6-(4-methoxy-phenyl)-1,2,3,4-tetrahydro-naphthalen-2-yl]-pyrrolidine

400 MHz ¹H NMR (CD₃OD) δ 7.5 (d, J=8.7 Hz, 2H), 7.3-7.4 (m, 2H), 7.2 (d, J=7.9 Hz, 1H), 7.0 (d, J=8.7 Hz, 2H), 5.4-5.6 (m, 1H), 3.9-4.1 (m, 2H), 3.5-3.7 (m, 3H), 3.3 (m, 4H), 3.0-3.1 (m, 3H), 2.3-2.6 (m, 3H), 1.9-2.0 (m, 1H); MS (M+1) 326.3.

### Example 46

### 1-[4-(6-Pyrrolidin-1-yl-5,6,7,8-tetrahydro-naphthalen-2-yl)-phenyl]-ethanone

400 MHz ¹H NMR (CD₃OD) δ 8.0-8.1 (d, J=8.7 Hz, 2H), 7.7 (d, J=8.3 Hz, 2H), 7.5 (d, J=8.3 Hz, 2H), 7.3 (J=7.5 Hz, 1H), 3.6 (m, 2H), 3.4-3.5 (dd, J=14.1, 7.1 Hz, 1H), 3.0-3.3 (m, 6H), 2.4-2.6 (m, 4H), 1.9-2.1 (m, 4H), 2.0 (m, 1H). MS (M+1) 320.4.

### Example 47

### 3,4-Difluoro-1-[6-(4-methoxy-phenyl)-1,2,3,4-tetrahydro-naphthalen-2-yl]-pyrrolidine

400 MHz ¹H NMR (CD₃OD) δ 7.5 (m, 2H), 7.4 (d, J=7.9 Hz, 1H), 7.3 (s, 1H), 7.2 (d, J=7.9 Hz, 1H), 7.0 (m, 2H), 5.6 (m, 1H), 5.5 (m, 1H), 3.8 (s, 3H), 3.7 (m, 1H), 3.2-3.3 (m, 6H), 3.0-3.1 (m, 2H), 2.4 (m, 1H), 2.0 (m, 1H). MS (M+1) 344.3.

### Example 48

### 1-[6-(4-Methoxy-phenyl)-1,2,3,4-tetrahydro-naphthalen-2-yl]-2-methyl-pyrrolidine

400 MHz ¹H NMR (CD₃OD) δ 7.5 (d, J=8.7 Hz, 2H), 7.3-7.4 (m, 2H), 7.2 (d, J=7.9 Hz, 1H), 7.0 (d, J=8.7 Hz, 2H), 3.8-3.9 (m, 1H), 3.7 (s, 3H), 3.6 (m, 1H), 3.3-3.4 (m, 3H), 3.0-3.1 (m, 3H), 2.3-2.4 (m, 2H), 2.0-2.1 (m, 2H), 1.8-2.0 (m, 2H), 1.5 (m, 3H). MS (M+1) 322.4.

### Example 49

### (R,R)-1-[6-(4-Methoxy-phenyl)-1,2,3,4-tetrahydro-naphthalen-2-yl]-2-methyl-pyrrolidine

400 MHz ¹H NMR (CD₃OD) δ 7.5 (d, J=8.7 Hz, 2H), 7.3-7.4 (m, 2H), 7.2 (d, J=7.9 Hz, 1H), 7.0 (d, J=8.7 Hz, 2H), 3.9 (m, 1H), 3.8 (s, 3H), 3.7 (m, 1H), 3.6 (m, 1H), 3.2-3.4 (m, 2H), 3.0-3.1 (m, 3H), 2.4 (m, 2H), 2.0-2.1 (m, 2H), 1.8-2.0 (m, 2H), 1.5 (dd, J=6.6, 2.1 Hz, 3H). MS (M+1) 322.4. Chiralcel OJ, Mobile Phase 85115, Heptane/EtOH, T_{R}=13.213 min.

### Example 50

### (S,R)-1-[6-(4-Methoxy-phenyl)-1,2,3,4-tetrahydro-naphthalen-2-yl]-2-methyl-pyrrolidine

Chiralcel OJ, Mobile Phase 85/15 Heptane/EtOH, T_{R}=13.659 min, MS (M+1) 322.4

### Example 51

### (R)-1-(6-bromo-1,2,3,4-tetrahydronaphthalen-2-yl)pyrrolidine

Chiralcel OD, Mobile Phase 80120 Heptane/IPA, T_{R}=9.378 min.

### Example 52

### (S)-1-(6-bromo-1,2,3,4-tetrahydronaphthalen-2-yl)pyrrolidine

Chiralcel OD, Mobile Phase 80120 Heptane/IPA, T_{R}=14.325 min.

### Example 53

### (R,R)-1-(6-Bromo-1,2,3,4-tetrahydro-naphthalen-2-yl)-2-methyl-pyrrolidine

Chiralcel OD, Mobile Phase 85/15 Heptane/IPO, T_{R}=19.591 min.

### Example 54

(S,R)-1-(6-Bromo-1,2,3,4-tetrahydro-naphthalen-2-yl)-2-methyl-pyrrolidine

400 MHz ¹H NMR (CDCl₃) δ 7.2 (m, 2H), 6.9 (d, J=7.9 Hz, 1H), 2.6-3.0 (m, 8H), 1.5-2.1 (m 5H), 1.4 (m, 1H), 1.0-1.1 (m, 3H). GCMS 293.0. Chiralcel OD, Mobile Phase 85/15 Heptane/IPO, T_{R}=24.109 min.

### Example 55

### (R,S)-1-(6-Bromo-1,2,3,4-tetrahydro-naphthalen-2-yl)-2-methyl-pyrrolidine

Chiralcel OD, Mobile Phase 85/15 Heptane/IPO, T_{R}=18.050 min.

### Example 56

### (S,S)-1-(6-Bromo-1,2,3,4-tetrahydro-naphthalen-2-yl)-2-methyl-pyrrolidine

400 MHz ¹H NMR (CDCl₃) δ 7.2 (m, 2H), 6.9 (d, J=7.9 Hz, 1H), 2.6-3.0 (m, 8H), 1.5-2.1 (m 5H), 1.4 (m, 1H), 1.0-1.1 (m, 3H). GCMS 293.0. Chiralcel OD, Mobile Phase 85/15 Heptane/IPO, T_{R}=20.109 min.

### Example 57

### (R)-N,N-Dimethyl-3-(6-pyrrolidin-1-yl-5,6,7,8-tetrahydro-naphthalen-2-yl)-benzamide

Chiralcel OJ, Mobile Phase 90/10 Heptane/EtOH, T_{R}=11.814 min. MS (M+1) 349.3.

### Example 58

### (R)-N,N-Dimethyl-3-(6-pyrrolidin-1-yl-5,6,7,8-tetrahydro-naphthalen-2-yl)-benzamide

Chiralcel OJ, Mobile Phase 90/10 Heptane/EtOH, T_{R}=13.677 min. MS (M+1) 349.3

### Example 59

### (S,R)-3-[6-(2-Methyl-Pyrrolidin-1-yl)-5,6,7,8-tetrahydro-naphthalen-2-yl]-pyridine

400 MHz ¹H NMR (CD₃OD) δ 9.0 (d, J=1.2 Hz, 1H), 8.6-8.7 (m, 2H), 7.9 (m, 1H), 7.6 (m, 2H), 7.4 (d, J=8.3 Hz, 1H), 3.9 (m, 1H), 3.8 (m, 1H), 3.6 (m, 1H), 3.4 (m, 1H), 3.0-3.2 (m, 4H), 2.3-2.4 (m, 2H), 1.8-2.1 (m, 4H), 1.5 (d, J=6.6 Hz, 3H). MS (M+1) 293.4. Chiralpak AD, Mobile Phase 85/15 Heptane/EtOH, T_{R}=8.512 min.

### Example 60

### (R,R)-4-[6-(2-Methyl-pyrrolidin-1-yl)-5,6,7,8-tetrahydro-naphthalen-2-yl]-pyridine

400 MHz ¹H NMR (CD₃OD) δ 9.0 (d, J=1.2 Hz, 1H), 8.6-8.7 (m, 2H), 7.9 (m, 1H), 7.6 (m, 2H), 7.4 (d, J=8.3 Hz, 1H), 3.9 (m, 1H), 3.8 (m, 1H), 3.6 (m, 1H), 3.4 (m, 1H), 3.0-3.2 (m, 4H), 2.3-2.4 (m, 2H), 1.8-2.1 (m, 4H), 1.5 (d, J=6.6 Hz, 3H). MS (M+1) 293.4. Chiralpak AD, Mobile Phase 85/15 Heptane/EtOH, T_{R}=6.445 min.

### Example 61

### 1-[6-(3,4-Dimethoxy-phenyl)-1,2,3,4-tetrahydro-naphthalen-2-yl]-2-methyl-pyrrolidine

400 MHz ¹H NMR (CD₃OD) δ 7.4 (m, 2H), 7.2 (d, J=7.9 Hz, 1H), 7.1 (m, 2H), 7.0 (d, J=8.7 Hz, 1H), 3.9 (s, 3H), 3.8 (s, 3H), 3.3-3.8 (m, 4H), 3.0 (m, 4H), 2.4 (m, 2H), 2.1 (m, 2H), 1.8-2.0 (m, 2H), 1.5 (m, 3H). MS (M+1) 352.1.

### Example 62

### 1-[6-(3-Fluoro-4-methoxy-phenyl)-1,2,3,4-tetrahydronaphthalen-2-yl]-2-methyl-pyrrolidine

400 MHz ¹H NMR (CD₃OD) δ 7.3-7.4 (m, 3H), 7.2 (d, 7.9 Hz, 1H), 7.1 (m, 2H), 3.9 (s, 3H), 3.8 (m, 1H), 3.6 (m, 1H), 3.2-3.4 (m, 2H), 3.0-3.1 (m, 4H), 2.3-2.4 (m, 2H), 1.8-2.2 (m, 4H), 1.5 (m, 3H). MS (M+1) 340.1.

### Example 63

### N-Methyl-4-(6-pyrrolidin-1-yl-5,6,7,8-tetrahydroaphthalen-2-yl)-benzamide

400 MHz ¹H NMR (CD₃OD) δ 7.8-7.9 (d, J=8.7 Hz, 2H), 7.7 id, J=8.7 Hz, 2H), 7.5 (m, 2H), 7.2-7.3 (d, J=7.9 Hz, 1H), 3.8 (m, 2H), 3.6 (m, 1H), 3.3-3.4 (dd, J=16.2, 4.2 Hz, 1H), 3.2-3.3 (m, 2H), 3.0-3.1 (m, 3H), 2.9 (s, 3H), 2.4 (m, 1H), 2.2 (m, 2H), 1.9-2.2 (m, 3H). MS (M+1) 335.4.

### Example 64

### 4-[6-(2-Methyl-pyrrolidin-1-yl)-5,6,7,8-tetrahydro-naphthalen-2-yl]-benzamide

400 MHz 7H NMR (CD₃OD) δ 8.0 (d, J=8.3 Hz, 2H), 7.7 (d, J=8.3 Hz, 2H), 7.4 (m, 2H), 7.2 (d, J=7.5 Hz, 1H), 2.8-3.5 (m, 8H), 2.3 (m, 1H), 2.2 (m, 1H), 2.0 (m, 2H), 1.8 (m, 1H), 1.6 (m, 1H), 1.3 (brs, 3H). MS (M+1) 335.4.

### Example 65

### 3-[6-(2-Methyl-pyrrolidin-1-yl)-5,6,7,8-tetrahydro-naphthalen-2-yl]-benzamide

400 MHz ¹H NMR (CD₃OD) δ 8.1-8.2 (m, 1H), 7.8-7.9 (m, 2H), 7.4-7.6 (m, 3H), 7.2-7.3 (m, 1H), 3.9 (m, 1H), 3.8 (m, 1H), 3.6 (m, 1H), 3.2-3.4 (m, 1H), 3.0-3.2 (m, 4H), 2.3-2.4 (m, 2H), 1.8-2.2 (m, 4H), 1.5 (dd, J=6.6, 2.9 Hz, 3H). MS (M+1) 335.4.

### Example 66

### (R,R)-3-[6-(2-Methyl-pyrrolidin-1-yl)-5,6,7,8-tetrahydro-naphthalen-2-yl]-benzamide

Chiralpak AD, Mobile Phase 85/15 Heptane/EtOH, T_{R}=10.934 min. MS (M+1) 335.4.

### Example 67

### (S,R)-3-[6-(2-Methyl-pyrrolidin-1-yl)-5,6,7,8-tetrahydro-naphthalen-2-yl]-benzamide

Chiralpak AD, Mobile Phase 85/15 Heptane/EtOH, T_{R}=14.219 min. MS (M+1) 335.4.

### Example 68

### 1-[6-(4-Methanesulfonyl-phenyl)-1,2,3,4-tetrahydro-naphthalen-2-yl]-2-methyl-pyrrolidine

400 MHz ¹H NMR (CD₃OD) δ 8.0 (d, J=8.3 Hz, 2H), 7.8-7.9 (d, J=8.7 Hz, 2H), 7.5 (m, 2H), 7.3 (d, J=7.9 Hz, 1H), 3.7-3.9 (m, 2H), 3.6 (m, 1H), 3.4 (m, 1H), 3.0-3.2 (m, 7H), 2.3-2.4 (m, 2H), 1.8-2.1 (m, 4H), 1.5 (m, 3H). MS (M+1) 369.3. GCMS 369.0.

The following tables of examples were also prepared according to the procedures and examples described above.

| | | Experimental | |
|---|---|---|---|
| | | MS | |
| Ex- | | Observed | MW |
| ample | IUPAC | (M + 1) | calc. |
| 69 | 1-[6-(2,5-Dimethyl-phenyl)-1,2,3,4- | 306.137 | 305.21 |
| | tetrahydro-naphthalen-2-yl]- | | |
| | pyrrolidine | | |
| 70 | 2-(6-Pyrrolidin-1-yl-5,6,7,8- | 306.139 | 305.18 |
| | tetrahydro-naphthalen-2-yl)- | | |
| | benzaldehyde | | |
| 71 | 1-[6-(2-Phenoxy-phenyl)-1,2,3,4- | 370.124 | 369.21 |
| | tetrahydro-naphthalen-2-yl]- | | |
| | pyrrolidine | | |
| 72 | 1-[6-(4-Fluoro-3-methyl-phenyl)- | 310.115 | 309.19 |
| | 1,2,3,4-tetrahydro-naphthalen-2-yl]- | | |
| | pyrrolidine | | |
| 73 | 1-[6-(4-Methoxy-3,5-dimethyl- | 336.176 | 335.22 |
| | phenyl)-1,2,3,4-tetrahydro- | | |
| | naphthalen-2-yl]-pyrrolidine | | |
| 74 | 1-[6-(3-Fluoro-4-methoxy-phenyl)- | 326.137 | 325.18 |
| | 1,2,3,4-tetrahydro-naphthalen-2-yl]- | | |
| | pyrrolidine | | |
| 75 | 1-[6-(2,3,4-Trimethoxy-phenyl)- | 368.131 | 367.21 |
| | 1,2,3,4-tetrahydro-naphthalen-2-yl]- | | |
| | pyrrolidine | | |
| 76 | 1-(6-Phenoxathiin-4-yl-1,2,3,4- | 400.12 | 399.17 |
| | tetrahydro-naphthalen-2-yl)- | | |
| | pyrrolidine | | |
| 77 | [3-(6-Pyrrolidin-1-yl-5,6,7,8- | 308.134 | 307.19 |
| | tetrahydro-naphthalen-2-yl)-phenyl]- | | |
| | methanol | | |
| 78 | 3-(6-Pyrrolidin-1-yl-5,6,7,8- | 303.104 | 302.18 |
| | tetrahydro-naphthalen-2-yl)- | | |
| | benzonitrile | | |
| 79 | 1-(6-Phenyl-1,2,3,4-tetrahydro- | 278.107 | 277.18 |
| | naphthalen-2-yl)-pyrrolidine | | |
| 80 | N,N-Diisopropyl-2-(6-pyrrolidin-1-yl- | 405.177 | 404.28 |
| | 5,6,7,8-tetrahydro-naphthalen-2-yl)- | | |
| | benzamide | | |
| 81 | 1-[6-(4-Methylsulfanyl-phenyl)- | 324.1 | 323.17 |
| | 1,2,3,4-tetrahydro-naphthalen-2-yl]- | | |
| | pyrrolidine | | |
| 82 | 1-(6-m-Tolyl-1,2,3,4-tetrahydro- | 292.137 | 291.2 |
| | naphthalen-2-yl)-pyrrolidine | | |
| 83 | 1-[6-(3-Chloro-4-fluoro-phenyl)- | 330.069 | 329.13 |
| | 1,2,3,4-tetrahydo-naphthalen-2-yl]- | | |
| | pyrrolidine | | |
| 84 | 1-[6-(3-Nitro-phenyl)-1,2,3,4- | 323.087 | 322.17 |
| | tetrahydro-naphthalen-2-yl]- | | |
| | pyrrolidine | | |
| 85 | 1-[6-(3,5-Dimethyl-phenyl)-1,2,3,4- | 306.141 | 305.21 |
| | tetrahydro-naphthalen-2-yl]- | | |
| | pyrrolidine | | |
| 86 | 1-[6-(3-Fluoro-phenyl)-1,2,3,4- | 296.109 | 295.17 |
| | tetrahydro-naphthalen-2-yl]- | | |
| | pyrrolidine | | |
| 87 | 1-[6-(3,4-Dichloro-phenyl)-1,2,3,4- | 346.063 | 345.11 |
| | tetrahydro-naphthalen-2-yl]- | | |
| | pyrrolidine | | |
| 88 | 1-[6-(2-Methoxy-phenyl)-1,2,3,4- | 308.12 | 307.19 |
| | tetrahydro-naphthalen-2-yl]- | | |
| | pyrrolidine | | |
| 89 | 1-[6-(3-Ethoxy-phenyl)-1,2,3,4- | 322.136 | 321.21 |
| | tetrahydro-naphthalen-2-yl]- | | |
| | pyrrolidine | | |
| 90 | 1-[6-(3,4-Difluoro-phenyl)-1,2,3,4- | 314.106 | 313.16 |
| | tetrahydro-naphthalen-2-yl]- | | |
| | pyrrolidine | | |
| 91 | 1-[6-(4-Fluoro-phenyl)-1,2,3,4- | 296.095 | 295.17 |
| | tetrahydro-naphthalen-2-yl]- | | |
| | pyrrolidine | | |
| 92 | 1-[6-(4-Trifluoromethoxy-phenyl)- | 362.117 | 361.17 |
| | 1,2,3,4-tetrahydro-naphthalen-2-yl]- | | |
| | pyrrolidine | | |
| 93 | 1-(6-Benzo[1,3]dioxol-5-yl-1,2,3,4- | 322.121 | 321.17 |
| | tetrahydro-naphthalen-2-yl)- | | |
| | pyrrolidine | | |
| 94 | 1-(5,6,7,8-Tetrahydro- | 328.128 | 327.2 |
| | [2,2′]binaphthalenyl-6-yl)-pyrrolidine | | |
| 95 | 1-[6-(2-Chloro-phenyl)-1,2,3,4- | 312.078 | 311.14 |
| | tetrahydro-naphthalen-2-yl]- | | |
| | pyrrolidine | | |
| 96 | 1-[6-(4-Ethyl-phenyl)-1,2,3,4- | 306.162 | 305.21 |
| | tetrahydro-naphthalen-2-yl]- | | |
| | pyrrolidine | | |
| 97 | 1-[6-(2-Ethoxy-phenyl)-1,2,3,4- | 322.148 | 321.21 |
| | tetrahydro-naphthalen-2-yl]- | | |
| | pyrrolidine | | |
| 98 | 1-[4-(6-Pyrrolidin-1-yl-5,6,7,8- | INPAT | 319.19 |
| | tetrahydro-naphthalen-2-yl)-phenyl]- | | |
| | ethanone | | |
| 99 | 1-[4-(6-Pyrrolidin-1-yl-5,6,7,8- | 320.143 | 319.19 |
| | tetrahydro-naphthalen-2-yl)-phenyl]- | | |
| | ethanone | | |
| 100 | 1-[6-(2,6-Dimethyl-phenyl)-1,2,3,4- | 306.161 | 305.21 |
| | tetrahydro-naphthalen-2-yl]- | | |
| | pyrrolidine | | |
| 101 | 1-[6-(4-Ethoxy-phenyl)-1,2,3,4- | 322.137 | 321.21 |
| | tetrahydro-naphthalen-2-yl]- | | |
| | pyrrolidine | | |
| 102 | 1-[3-(6-Pyrrolidin-1-yl-5,6,7,8- | 320.148 | 319.19 |
| | tetrahydro-naphthalen-2-yl)-phenyl]- | | |
| | ethanone | | |
| 103 | 1-[6-(2-Methoxy-5-methyl-phenyl)- | 322.151 | 321.21 |
| | 1,2,3,4-tetrahydro-naphthalen-2-yl]- | | |
| | pyrrolidine | | |
| 104 | Dimethyl-[4-(6-pyrrolidin-1-yl-5,6,7,8- | 161.077 | 320.23 |
| | tetrahydro-naphthalen-2-yl)-phenyl]- | | |
| | amine | | |
| 105 | [4-(6-Pyrrolidin-1-yl-5,6,7,8- | 308.141 | 307.19 |
| | tetrahydro-naphthalen-2-yl)-phenyl]- | | |
| | methanol | | |
| 106 | 1-[6-(2-Fluoro-3-methoxy-phenyl)- | 326.1 | 325.18 |
| | 1,2,3,4-tetrahydro-naphthalen-2-yl]- | | |
| | pyrrolidine | | |
| 107 | [2-(6-Pyrrolidin-1-yl-5,6,7,8- | 308.135 | 307.19 |
| | tetrahydro-naphthalen-2-yl)-phenyl]- | | |
| | methanol | | |
| 108 | 1-(4-Methyl-5′,6′,7′,8′-tetrahydro- | 342.129 | 341.21 |
| | [1,2′]binaphthalenyl-6′-yl)-pyrrolidine | | |
| 109 | N-[3-(6-Pyrrolidin-1-yl-5,6,7,8- | 335.142 | 334.2 |
| | tetrahydro-naphthalen-2-yl)-phenyl]- | | |
| | acetamide | | |
| 110 | 3-(6-Pyrrolidin-1-yl-5,6,7,8- | 350.162 | 349.2 |
| | tetrahydro-naphthalen-2-yl)-benzoic | | |
| | acid ethyl ester | | |
| 111 | 1-[6-(2-Benzyloxy-phenyl)-1,2,3,4- | 384.129 | 383.22 |
| | tetrahydro-naphthalen-2-yl]- | | |
| | pyrrolidine | | |
| 112 | 1-[6-(3-Methylsulfanyl-phenyl)- | 324.088 | 323.17 |
| | 1,2,3,4-tetrahydro-naphthalen-2-yl]- | | |
| | pyrrolidine | | |
| 113 | 1-[6-(4-Methoxy-3-methyl-phenyl)- | 322.122 | 321.21 |
| | 1,2,3,4-tetrahydro-naphthalen-2-yl]- | | |
| | pyrrolidine | | |
| 114 | 4-(6-Pyrrolidin-1-yl-5,6,7,8- | 303.11 | 302.18 |
| | tetrahydro-naphthalen-2-yl)- | | |
| | benzonitrile | | |
| 115 | 1-[6-(3,4-Dimethyl-phenyl)-1,2,3,4- | 306.132 | 305.21 |
| | tetrahydro-naphthalen-2-yl]- | | |
| | pyrrolidine | | |
| 116 | 1-(6-Biphenyl-2-yl-1,2,3,4-tetrahydro- | 354.123 | 353.21 |
| | naphthalen-2-yl)-pyrrolidine | | |
| 117 | 1-[6-(4-Benzyloxy-phenyl)-1,2,3,4- | 384.132 | 383.22 |
| | tetrahydro-naphthalen-2-yl]- | | |
| | pyrrolidine | | |
| 118 | 1-[6-(2-Fluoro-biphenyl-4-yl)-1,2,3,4- | 372.114 | 371.2 |
| | tetrahydro-naphthalen-2-yl]- | | |
| | pyrrolidine | | |
| 119 | 1-[6-(3,4,5-Trimethoxy-phenyl)- | 368.122 | 367.21 |
| | 1,2,3,4-tetrahydro-naphthalen-2-yl]- | | |
| | pyrrolidine | | |
| 120 | 1-[6-(3-Benzyloxy-phenyl)-1,2,3,4- | 384.134 | 383.22 |
| | tetrahydro-naphthalen-2-yl]- | | |
| | pyrrolidine | | |
| 121 | 1-[6-(4-Fluoro-2-methyl-phenyl)- | 310.103 | 309.19 |
| | 1,2,3,4-tetrahydro-naphthalen-2-yl]- | | |
| | pyrrolidine | | |
| 122 | 1-[6-(2-Ethyl-phenyl)-1,2,3,4- | 306.167 | 305.21 |
| | tetrahydro-naphthalen-2-yl]- | | |
| | pyrrolidine | | |
| 123 | 4-(6-Pyrrolidin-1-yl-5,6,7,8- | 294.101 | 293.18 |
| | tetrahydro-naphthalen-2-yl)-phenol | | |
| 124 | 1-[6-(2-Methylsulfanyl-phenyl)- | 324.101 | 323.17 |
| | 1,2,3,4-tetrahydro-naphthalen-2-yl]- | | |
| | pyrrolidine | | |
| 125 | 1-[6-(4-Benzyloxy-2-fluoro-phenyl)- | 402.13 | 401.22 |
| | 1,2,3,4-tetrahydro-naphthalen-2-yl]- | | |
| | pyrrolidine | | |
| 126 | 1-[6-(4-Isopropoxy-phenyl)-1,2,3,4- | 336.162 | 335.22 |
| | tetrahydro-naphthalen-2-yl]- | | |
| | pyrrolidine | | |
| 127 | 3,5-Dimethyl-1-[3-(6-pyrrolidin-1-yl- | 186.585 | 371.24 |
| | 5,6,7,8-tetrahydro-naphthalen-2-yl)- | | |
| | phenyl]-1H-pyrazole | | |
| 128 | 1-(5′,6′,7′,8′-Tetrahydro- | 328.112 | 327.2 |
| | [1,2′]binaphthalenyl-6′-yl)-pyrrolidine | | |
| 129 | 1-[6-(2,3-Dihydro-benzo[1,4]dioxin-6- | 336.102 | 335.19 |
| | yl)-1,2,3,4-tetrahydro-naphthalen-2- | | |
| | yl]-pyrrolidine | | |
| 130 | 1-{6-[4-(1-Methoxy-ethyl)-phenyl]- | 336.166 | 335.22 |
| | 1,2,3,4-tetrahydro-naphthalen-2-yl}- | | |
| | pyrrolidine | | |
| 131 | 1-[6-(3-Chloro-phenyl)-1,2,3,4- | 312.062 | 311.14 |
| | tetrahydro-naphthalen-2-yl]- | | |
| | pyrrolidine | | |
| 132 | 1-[6-(2,3-Dihydro-benzofuran-5-yl)- | 320.137 | 319.19 |
| | 1,2,3,4-tetrahydro-naphthalen-2-yl]- | | |
| | pyrrolidine | | |
| 133 | 1-[3-(6-Pyrrolidin-1-yl-5,6,7,8- | 344.113 | 343.2 |
| | tetrahydro-naphthalen-2-yl)-phenyl]- | | |
| | 1H-pyrazole | | |
| 134 | 1-[6-(3,4-Dihydro-2H- | 350.111 | 349.2 |
| | benzo[b][1,4]dioxepin-7-yl)-1,2,3,4- | | |
| | tetrahydro-naphthalen-2-yl]- | | |
| | pyrrolidine | | |
| 135 | 1-[6-(3-Methoxy-phenyl)-1,2,3,4- | 308.146 | 307.19 |
| | tetrahydro-naphthalen-2-yl]- | | |
| | pyrrolidine | | |
| 136 | 2-Isopropyl-5-(6-pyrrolidin-1-yl- | 181.08 | 360.26 |
| | 5,6,7,8-tetrahydro-naphthalen-2-yl)- | | |
| | 2,3-dihydro-1H-isoindole | | |
| 137 | 1-[6-(4-Chloro-phenyl)-1,2,3,4- | 312.1 | 311.14 |
| | tetrahydro-naphthalen-2-yl]- | | |
| | pyrrolidine | | |
| 138 | 1-[6-(5-Chloro-2-fluoro-phenyl)- | 330.074 | 329.13 |
| | 1,2,3,4-tetrahydro-naphthalen-2-yl]- | | |
| | pyrrolidine | | |
| 139 | 1-[6-(5-Chloro-2-methoxy-phenyl)- | 356.028 | 355.17 |
| | 1,2,3,4-tetrahydro-naphthalen-2-yl]- | | |
| | 2-methyl-pyrrolidine | | |
| 140 | 4-Methoxy-3-[6-(2-methyl-pyrrolidin- | 350.056 | 349.2 |
| | 1-yl)-5,6,7,8-tetrahydro-naphthalen- | | |
| | 2-yl]-benzaldehyde | | |
| 141 | 3-[6-(2-Methyl-pyrrolidin-1-yl)- | 317.064 | 316.19 |
| | 5,6,7,8-tetrahydro-naphthalen-2-yl]- | | |
| | benzonitrile | | |
| 142 | 1-(6-Biphenyl-3-yl-1,2,3,4-tetrahydro- | 368.078 | 367.23 |
| | naphthalen-2-yl)-2-methyl-pyrrolidine | | |
| 143 | N-tert-Butyl-2-[6-(2-methyl-pyrrolidin- | 427.061 | 426.23 |
| | 1-yl)-5,6,7,8-tetrahydro-naphthalen- | | |
| | 2-yl]-benzenesulfonamide | | |
| 144 | 2-Methyl-1-(6-phenyl-1,2,3,4- | 292.071 | 291.2 |
| | tetrahydro-naphthalen-2-yl)- | | |
| | pyrrolidine | | |
| 145 | 1-[6-(2,5-Dimethoxy-phenyl)-1,2,3,4- | 352.073 | 351.22 |
| | tetrahydro-naphthalen-2-yl]-2- | | |
| | methyl-pyrrolidine | | |
| 146 | 2-Methyl-1-(6-thianthren-1-yl-1,2,3,4- | 429.99 | 429.16 |
| | tetrahydro-naphthalen-2-yl)- | | |
| | pyrrolidine | | |
| 147 | 2-Methyl-1-(6-p-tolyl-1,2,3,4- | 306.079 | 305.21 |
| | tetrahydro-naphthalen-2-yl)- | | |
| | pyrrolidine | | |
| 148 | 2-Methyl-1-[6-(3-nitro-phenyl)- | 337.043 | 336.18 |
| | 1,2,3,4-tetrahydro-naphthalen-2-yl]- | | |
| | pyrrolidine | | |
| 149 | 1-[6-(5-Fluoro-2-methoxy-phenyl)- | 340.051 | 339.2 |
| | 1,2,3,4-tetrahydro-naphthalen-2-yl]- | | |
| | 2-methyl-pyrrolidine | | |
| 150 | N,N-Diisopropyl-2-methoxy-6-[6-(2- | 449.133 | 448.31 |
| | methyl-pyrrolidin-1-yl)-5,6,7,8- | | |
| | tetrahydro-naphthalen-2-yl]- | | |
| | benzamide | | |
| 151 | 1-[6-(2,6-Dimethoxy-phenyl)-1,2,3,4- | 352.073 | 351.22 |
| | tetrahydro-naphthalen-2-yl]-2- | | |
| | methyl-pyrrolidine | | |
| 152 | N,N-Diisopropyl-2-[6-(2-methyl- | 419.122 | 418.3 |
| | pyrrolidin-1-yl)-5,6,7,8-tetrahydro- | | |
| | naphthalen-2-yl]-benzamide | | |
| 153 | 1-[6-(4-Fluoro-phenyl)-1,2,3,4- | 310.053 | 309.19 |
| | tetrahydro-naphthalen-2-yl]-2- | | |
| | methyl-pyrrolidine | | |
| 154 | 1-[6-(3,5-Bis-trifluoromethyl-phenyl)- | 427.998 | 427.17 |
| | 1,2,3,4-tetrahydro-naphthalen-2-yl]- | | |
| | 2-methyl-pyrrolidine | | |
| 155 | 2-Methyl-1-(6-m-tolyl-1,2,3,4- | 306.083 | 305.21 |
| | tetrahydro-naphthalen-2-yl)- | | |
| | pyrrolidine | | |
| 156 | 1-{6-[4-(4-Methoxy-phenoxy)- | 414.069 | 413.24 |
| | phenyl]-1,2,3,4-tetrahydro- | | |
| | naphthalen-2-yl}-2-methyl-pyrrolidine | | |
| 157 | 1-[6-(3-Chloro-4-fluoro-phenyl)- | 344.007 | 343.15 |
| | 1,2,3,4-tetrahydro-naphthalen-2-yl]- | | |
| | 2-methyl-pyrrolidine | | |
| 158 | 2-Methyl-1-[6-(4-trifluoromethyl- | 360.041 | 359.19 |
| | phenyl)-1,2,3,4-tetrahydro- | | |
| | naphthalen-2-yl]-pyrrolidine | | |
| 159 | 2-Methyl-1-[6-(2,3,4-trimethoxy- | 382.069 | 381.23 |
| | phenyl)-1,2,3,4-tetrahydro- | | |
| | naphthalen-2-yl]-pyrrolidine | | |
| 160 | 1-[6-(3,5-Dichloro-phenyl)-1,2,3,4- | 359.98 | 359.12 |
| | tetrahydro-naphthalen-2-yl]-2- | | |
| | methyl-pyrrolidine | | |
| 161 | 2-Methyl-1-(6-o-tolyl-1,2,3,4- | 306.084 | 305.21 |
| | tetrahydro-naphthalen-2-yl)- | | |
| | pyrrolidine | | |
| 162 | 1-[6-(2-Methoxy-phenyl)-1,2,3,4- | 322.084 | 321.21 |
| | tetrahydro-naphthalen-2-yl]-2- | | |
| | methyl-pyrrolidine | | |
| 163 | 2-Methyl-1-(6-thiophen-3-yl-1,2,3,4- | 298.034 | 297.16 |
| | tetrahydro-naphthalen-2-yl)- | | |
| | pyrrolidine | | |
| 164 | 2-Methyl-1-[6-(4-phenoxy-phenyl)- | 384.074 | 383.22 |
| | 1,2,3,4-tetrahydro-naphthalen-2-yl]- | | |
| | pyrrolidine | | |
| 165 | 1-[6-(3,5-Dimethyl-phenyl)-1,2,3,4- | 320.099 | 319.23 |
| | tetrahydro-naphthalen-2-yl]-2- | | |
| | methyl-pyrrolidine | | |
| 166 | 2-Methyl-1-(5,6,7,8-tetrahydro- | 342.085 | 341.21 |
| | [2,2′]binaphthalenyl-6-yl)-pyrrolidine | | |
| 167 | 1-[6-(2-Ethoxy-phenyl)-1,2,3,4- | 336.092 | 335.22 |
| | tetrahydro-naphthalen-2-yl]-2- | | |
| | methyl-pyrrolidine | | |
| 168 | 2-Methyl-1-[6-(3-trifluoromethyl- | 360.042 | 359.19 |
| | phenyl)-1,2,3,4-tetrahydro- | | |
| | naphthalen-2-yl]-pyrrolidine | | |
| 169 | 1-[6-(3-Ethoxy-phenyl)-1,2,3,4- | 336.096 | 335.22 |
| | tetrahydro-naphthalen-2-yl]-2- | | |
| | methyl-pyrrolidine | | |
| 170 | 1-[6-(3,4-Difluoro-phenyl)-1,2,3,4- | 328.066 | 327.18 |
| | tetrahydro-naphthalen-2-yl]-2- | | |
| | methyl-pyrrolidine | | |
| 171 | 1-[6-(3-Fluoro-phenyl)-1,2,3,4- | 310.066 | 309.19 |
| | tetrahydro-naphthalen-2-yl]-2- | | |
| | methyl-pyrrolidine | | |
| 172 | 2-Methyl-1-[6-(2-trifluoromethyl- | 360.044 | 359.19 |
| | phenyl)-1,2,3,4-tetrahydro- | | |
| | naphthalen-2-yl]-pyrrolidine | | |
| 173 | 1-[6-(4-Ethyl-phenyl)-1,2,3,4- | 320.103 | 319.23 |
| | tetrahydro-naphthalen-2-yl]-2- | | |
| | methyl-pyrrolidine | | |
| 174 | 1-[6-(2-Chloro-phenyl)-1,2,3,4- | 326.033 | 325.16 |
| | tetrahydro-naphthalen-2-yl]-2- | | |
| | methyl-pyrrolidine | | |
| 175 | 1-[6-(3,4-Dichloro-phenyl)-1,2,3,4- | 360.002 | 359.12 |
| | tetrahydro-naphthalen-2-yl]-2- | | |
| | methyl-pyrrolidine | | |
| 176 | 1-(6-Benzo[1,3]dioxol-5-yl-1,2,3,4- | 336.073 | 335.19 |
| | tetrahydro-naphthalen-2-yl)-2- | | |
| | methyl-pyrrolidine | | |
| 177 | 1-[6-(4-Ethoxy-phenyl)-1,2,3,4- | 336.103 | 335.22 |
| | tetrahydro-naphthalen-2-yl]-2- | | |
| | methyl-pyrrolidine | | |
| 178 | 1-[6-(2-Fluoro-phenyl)-1,2,3,4- | 310.064 | 309.19 |
| | tetrahydro-naphthalen-2-yl]-2- | | |
| | methyl-pyrrolidine | | |
| 179 | 1-{3-[6-(2-Methyl-pyrrolidin-1-yl)- | 334.097 | 333.21 |
| | 5,6,7,8-tetrahydro-naphthalen-2-yl]- | | |
| | phenyl}-ethanone | | |
| 180 | {4-[6-(2-Methyl-pyrrolidin-1-yl)- | 322.091 | 321.21 |
| | 5,6,7,8-tetrahydro-naphthalen-2-yl]- | | |
| | phenyl}-methanol | | |
| 181 | 1-[6-(4-tert-Butyl-phenyl)-1,2,3,4- | 348.134 | 347.26 |
| | tetrahydro-naphthalen-2-yl]-2- | | |
| | methyl-pyrrolidine | | |
| 182 | 1-[6-(2,6-Dimethyl-phenyl)-1,2,3,4- | 320.108 | 319.23 |
| | tetrahydro-naphthalen-2-yl]-2- | | |
| | methyl-pyrrolidine | | |
| 183 | 2-Methyl-1-[6-(4-trifluoromethoxy- | 376.049 | 375.18 |
| | phenyl)-1,2,3,4-tetrahydro- | | |
| | naphthalen-2-yl]-pyrrolidine | | |
| 184 | 1-[6-(2,5-Difluoro-phenyl)-1,2,3,4- | 328.065 | 327.18 |
| | tetrahydro-naphthalen-2-yl]-2- | | |
| | methyl-pyrrolidine | | |
| 185 | 1-[6-(4-Fluoro-3-methyl-phenyl)- | 324.1 | 323.2 |
| | 1,2,3,4-tetrahydro-naphthalen-2-yl]- | | |
| | 2-methyl-pyrrolidine | | |
| 186 | {3-[6-(2-Methyl-pyrrolidin-1-yl)- | 322.103 | 321.21 |
| | 5,6,7,8-tetrahydro-naphthalen-2-yl]- | | |
| | phenyl}-methanol | | |
| 187 | 1-[6-(2,4-Difluoro-phenyl)-1,2,3,4- | 328.062 | 327.18 |
| | tetrahydro-naphthalen-2-yl]-2- | | |
| | methyl-pyrrolidine | | |
| 188 | 1-[6-(4-Methoxy-3,5-dimethyl- | 350.128 | 349.24 |
| | phenyl)-1,2,3,4-tetrahydro- | | |
| | naphthalen-2-yl]-2-methyl-pyrrolidine | | |
| 189 | 1-[6-(2,3-Dimethyl-phenyl)-1,2,3,4- | 320.12 | 319.23 |
| | tetrahydro-naphthalen-2-yl]-2- | | |
| | methyl-pyrrolidine | | |
| 190 | 2-Methyl-1-[6-(2-trifluoromethoxy- | 376.057 | 375.18 |
| | phenyl)-1,2,3,4-tetrahydro- | | |
| | naphthalen-2-yl]-pyrrolidine | | |
| 191 | 2-Methyl-1-[6-(3-trifluoromethoxy- | 376.053 | 375.18 |
| | phenyl)-1,2,3,4-tetrahydro- | | |
| | naphthalen-2-yl]-pyrrolidine | | |
| 192 | 1-[6-(4-Isobutyl-phenyl)-1,2,3,4- | 348.143 | 347.26 |
| | tetrahydro-naphthalen-2-yl]-2- | | |
| | methyl-pyrrolidine | | |
| 193 | 2-Methyl-1-[6-(2-phenoxy-phenyl)- | 384.1 | 383.22 |
| | 1,2,3,4-tetrahydro-naphthalen-2-yl]- | | |
| | pyrrolidine | | |
| 194 | 1-[6-(3,5-Difluoro-phenyl)-1,2,3,4- | 328.067 | 327.18 |
| | tetrahydro-naphthalen-2-yl]-2- | | |
| | methyl-pyrrolidine | | |
| 195 | 4-[6-(2-Methyl-pyrrolidin-1-yl)- | 378.112 | 377.24 |
| | 5,6,7,8-tetrahydro-naphthalen-2-yl]- | | |
| | benzoic acid isopropyl ester | | |
| 196 | 1-[6-(2,5-Dimethyl-phenyl)-1,2,3,4- | 320.12 | 319.23 |
| | tetrahydro-naphthalen-2-yl]-2- | | |
| | methyl-pyrrolidine | | |
| 197 | 1-{2-[6-(2-Methyl-pyrrolidin-1-yl)- | 334.102 | 333.21 |
| | 5,6,7,8-tetrahydro-naphthalen-2-yl]- | | |
| | phenyl}-ethanone | | |
| 198 | 4-[6-(2-Methyl-pyrrolidin-1-yl)- | 335.096 | 334.2 |
| | 5,6,7,8-tetrahydro-naphthalen-2-yl]- | | |
| | benzamide | | |
| 199 | 3-[6-(2-Methyl-pyrrolidin-1-yl)- | 308.094 | 307.19 |
| | 5,6,7,8-tetrahydro-naphthalen-2-yl]- | | |
| | phenol | | |
| 200 | 2-Methyl-1-(4-methyl-5′,6′,7′,8′- | 356.136 | 355.23 |
| | tetrahydro-[1,2′]binaphthalenyl-6′-yl)- | | |
| | pyrrolidine | | |
| 201 | 1-[6-(2-Benzyloxy-phenyl)-1,2,3,4- | 398.135 | 397.24 |
| | tetrahydro-naphthalen-2-yl]-2- | | |
| | methyl-pyrrolidine | | |
| 202 | 3-[6-(2-Methyl-pyrrolidin-1-yl)- | 293.115 | 292.19 |
| | 5,6,7,8-tetrahydro-naphthalen-2-yl]- | | |
| | pyridine | | |
| 203 | {2-[6-(2-Methyl-pyrrolidin-1-yl)- | 322.121 | 321.21 |
| | 5,6,7,8-tetrahydro-naphthalen-2-yl]- | | |
| | phenyl}-methanol | | |
| 204 | 1-[6-(2-Fluoro-3-methoxy-phenyl)- | 340.106 | 339.2 |
| | 1,2,3,4-tetrahydro-naphthalen-2-yl]- | | |
| | 2-methyl-pyrrolidine | | |
| 205 | 3-[6-(2-Methyl-pyrrolidin-1-yl)- | 364.121 | 363.22 |
| | 5,6,7,8-tetrahydro-naphthalen-2-yl]- | | |
| | benzoic acid ethyl ester | | |
| 206 | 1-(6-Furan-3-yl-1,2,3,4-tetrahydro- | 282.095 | 281.18 |
| | naphthalen-2-yl)-2-methyl-pyrrolidine | | |
| 207 | 2-Methyl-1-[6-(3-methylsulfanyl- | 338.086 | 337.19 |
| | phenyl)-1,2,3,4-tetrahydro- | | |
| | naphthalen-2-yl]-pyrrolidine | | |
| 208 | 1-[6-(4-Methoxy-3-methyl-phenyl)- | 336.132 | 335.22 |
| | 1,2,3,4-tetrahydro-naphthalen-2-yl]- | | |
| | 2-methyl-pyrrolidine | | |
| 209 | {4-[6-(2-Methyl-pyrrolidin-1-yl)- | 441.14 | 440.25 |
| | 5,6,7,8-tetrahydro-naphthalen-2-yl]- | | |
| | phenyl}-carbamic acid benzyl ester | | |
| 210 | 2-Methyl-1-[6-(5-methyl-furan-2-yl)- | 293.998 | 295.19 |
| | 1,2,3,4-tetrahydro-naphthalen-2-yl]- | | |
| | pyrrolidine | | |
| 211 | 4-[6-(2-Methyl-pyrrolidin-1-yl)- | 364.116 | 363.22 |
| | 5,6,7,8-tetrahydro-naphthalen-2-yl]- | | |
| | benzoic acid ethyl ester | | |
| 212 | 2-Methoxy-5-[6-(2-methyl-pyrrolidin- | 323.118 | 322.2 |
| | 1-yl)-5,6,7,8-tetrahydro-naphthalen- | | |
| | 2-yl]-pyridine | | |
| 213 | 1-(6-Biphenyl-2-yl-1,2,3,4-tetrahydro- | 368.123 | 367.23 |
| | naphthalen-2-yl)-2-methyl-pyrrolidine | | |
| 214 | N-{3-[6-(2-Methyl-pyrrolidin-1-yl)- | 349.118 | 348.22 |
| | 5,6,7,8-tetrahydro-naphthalen-2-yl]- | | |
| | phenyl}-acetamide | | |
| 215 | 1-[6-(2,6-Dichloro-phenyl)-1,2,3,4- | 360.026 | 359.12 |
| | tetrahydro-naphthalen-2-yl]-2- | | |
| | methyl-pyrrolidine | | |
| 216 | 1-(6-Dibenzothiophen-1-yl-1,2,3,4- | 398.08 | 397.19 |
| | tetrahydro-naphthalen-2-yl)-2- | | |
| | methyl-pyrrolidine | | |
| 217 | 1-[6-(2-Methoxy-5-methyl-phenyl)- | 336.127 | 335.22 |
| | 1,2,3,4-tetrahydro-naphthalen-2-yl]- | | |
| | 2-methyl-pyrrolidine | | |
| 218 | 2-Methyl-1-[6-(3,4,5-trimethoxy- | 382.127 | 381.23 |
| | phenyl)-1,2,3,4-tetrahydro- | | |
| | naphthalen-2-yl]-pyrrolidine | | |
| 219 | 1-[6-(4-Benzyloxy-3-fluoro-phenyl)- | 416.117 | 415.23 |
| | 1,2,3,4-tetrahydro-naphthalen-2-yl]- | | |
| | 2-methyl-pyrrolidine | | |
| 220 | 1-[6-(3,4-Dimethyl-phenyl)-1,2,3,4- | 320.135 | 319.23 |
| | tetrahydro-naphthalen-2-yl]-2- | | |
| | methyl-pyrrolidine | | |
| 221 | 4-[6-(2-Methyl-pyrrolidin-1-yl)- | 426.128 | 425.24 |
| | 5,6,7,8-tetrahydro-naphthalen-2-yl]- | | |
| | benzoic acid benzyl ester | | |
| 222 | 1-[6-(2-Fluoro-biphenyl-4-yl)-1,2,3,4- | 386.118 | 385.22 |
| | tetrahydro-naphthalen-2-yl]-2- | | |
| | methyl-pyrrolidine | | |
| 223 | 4-[6-(2-Methyl-pyrrolidin-1-yl)- | 317.105 | 316.19 |
| | 5,6,7,8-tetrahydro-naphthalen-2-yl]- | | |
| | benzonitrile | | |
| 224 | 1-[6-(4-Isopropyl-phenyl)-1,2,3,4- | 334.15 | 333.25 |
| | tetrahydro-naphthalen-2-yl]-2- | | |
| | methyl-pyrrolidine | | |
| 225 | 1-[6-(2,3-Difluoro-phenyl)-1,2,3,4- | 328.089 | 327.18 |
| | tetrahydro-naphthalen-2-yl]-2- | | |
| | methyl-pyrrolidine | | |
| 226 | 1-[6-(5-Isopropyl-2-methoxy-phenyl)- | 364.155 | 363.26 |
| | 1,2,3,4-tetrahydro-naphthalen-2-yl]- | | |
| | 2-methyl-pyrrolidine | | |
| 227 | 2-Methyl-1-[6-(4-pentyl-phenyl)- | 362.177 | 361.28 |
| | 1,2,3,4-tetrahydro-naphthalen-2-yl]- | | |
| | pyrrolidine | | |
| 228 | 1-[6-(2,3-Dichloro-phenyl)-1,2,3,4- | 360.028 | 359.12 |
| | tetrahydro-naphthalen-2-yl]-2- | | |
| | methyl-pyrrolidine | | |
| 229 | 1-[6-(4-Methanesulfonyl-phenyl)- | 370.078 | 369.18 |
| | 1,2,3,4-tetrahydro-naphthalen-2-yl]- | | |
| | 2-methyl-pyrrolidine | | |
| 230 | 4-[6-(2-Methyl-pyrrolidin-1-yl)- | 308.107 | 307.19 |
| | 5,6,7,8-tetrahydro-naphthalen-2-yl]- | | |
| | phenol | | |
| 231 | 2-Methyl-1-[6-(2-methylsulfanyl- | 338.091 | 337.19 |
| | phenyl)-1,2,3,4-tetrahydro- | | |
| | naphthalen-2-yl]-pyrrolidine | | |
| 232 | 1-[6-(2,4-Bis-trifluoromethyl-phenyl)- | 428.059 | 427.17 |
| | 1,2,3,4-tetrahydro-naphthalen-2-yl]- | | |
| | 2-methyl-pyrrolidine | | |
| 233 | 1-[6-(3-Benzyloxy-phenyl)-1,2,3,4- | 398.14 | 397.24 |
| | tetrahydro-naphthalen-2-yl]-2- | | |
| | methyl-pyrrolidine | | |
| 234 | 1-[6-(4-Fluoro-2-methyl-phenyl)- | 324.113 | 323.2 |
| | 1,2,3,4-tetrahydro-naphthalen-2-yl]- | | |
| | 2-methyl-pyrrolidine | | |
| 235 | 3-[6-(2-Methyl-pyrrolidin-1-yl)- | 336.1 | 335.19 |
| | 5,6,7,8-tetrahydro-naphthalen-2-yl]- | | |
| | benzoic acid | | |
| 236 | 1-[6-(2-Ethyl-phenyl)-1,2,3,4- | 320.142 | 319.23 |
| | tetrahydro-naphthalen-2-yl]-2- | | |
| | methyl-pyrrolidine | | |
| 237 | 1-[6-(4-Benzyloxy-phenyl)-1,2,3,4- | 398.139 | 397.24 |
| | tetrahydro-naphthalen-2-yl]-2- | | |
| | methyl-pyrrolidine | | |
| 238 | 1-[6-(4-Benzyloxy-2-fluoro-phenyl)- | 416.124 | 415.23 |
| | 1,2,3,4-tetrahydro-naphthalen-2-yl]- | | |
| | 2-methyl-pyrrolidine | | |
| 239 | 1-[6-(4-Butyl-phenyl)-1,2,3,4- | 348.172 | 347.26 |
| | tetrahydro-naphthalen-2-yl]-2- | | |
| | methyl-pyrrolidine | | |
| 240 | 2-Methyl-1-(5′,6′,7′,8′-tetrahydro- | 342.124 | 341.21 |
| | [1,2′]binaphthalenyl-6′-yl)-pyrrolidine | | |
| 241 | 1-[6-(3-Chloro-phenyl)-1,2,3,4- | 326.073 | 325.16 |
| | tetrahydro-naphthalen-2-yl]-2- | | |
| | methyl-pyrrolidine | | |
| 242 | 1-[6-(4-Ethanesulfonyl-phenyl)- | 384.1 | 383.19 |
| | 1,2,3,4-tetrahydro-naphthalen-2-yl]- | | |
| | 2-methyl-pyrrolidine | | |
| 243 | 1-[6-(2,4-Dichloro-phenyl)-1,2,3,4- | 360.034 | 359.12 |
| | tetrahydro-naphthalen-2-yl]-2- | | |
| | methyl-pyrrolidine | | |
| 244 | 1-{3-[6-(2-Methyl-pyrrolidin-1-yl)- | 358.124 | 357.22 |
| | 5,6,7,8-tetrahydro-naphthalen-2-yl]- | | |
| | phenyl}-1H-pyrazole | | |
| 245 | 3-[6-(2-Methyl-pyrrolidin-1-yl)- | 335.112 | 334.2 |
| | 5,6,7,8-tetrahydro-naphthalen-2-yl]- | | |
| | benzamide | | |
| 246 | 1-(6-Dibenzofuran-4-yl-1,2,3,4- | 382.109 | 381.21 |
| | tetrahydro-naphthalen-2-yl)-2- | | |
| | methyl-pyrrolidine | | |
| 247 | 1-[6-(4-Chloro-phenyl)-1,2,3,4- | 326.075 | 325.16 |
| | tetrahydro-naphthalen-2-yl]-2- | | |
| | methyl-pyrrolidine | | |
| 248 | 1-[6-(4-Isopropoxy-phenyl)-1,2,3,4- | 350.144 | 349.24 |
| | tetrahydro-naphthalen-2-yl]-2- | | |
| | methyl-pyrrolidine | | |
| 249 | 3-Chloro-4-[6-(2-methyl-pyrrolidin-1- | 327.068 | 326.15 |
| | yl)-5,6,7,8-tetrahydro-naphthalen-2- | | |
| | yl]-pyridine | | |
| 250 | 1-[6-(3-Methoxy-phenyl)-1,2,3,4- | 322.115 | 321.21 |
| | tetrahydro-naphthalen-2-yl]-2- | | |
| | methyl-pyrrolidine | | |
| 251 | 2-Methyl-1-[6-(4-methyl-3-nitro- | 351.108 | 350.2 |
| | phenyl)-1,2,3,4-tetrahydro- | | |
| | naphthalen-2-yl]-pyrrolidine | | |
| 252 | 2-Methyl-1-(6-methyl-1,2,3,4- | 230.113 | 229.18 |
| | tetrahydro-naphthalen-2-yl)- | | |
| | pyrrolidine | | |
| 253 | 1-[6-(3,4-Dihydro-2H- | 364.115 | 363.22 |
| | benzo[b][1,4]dioxepin-7-yl)-1,2,3,4- | | |
| | tetrahydro-naphthalen-2-yl]-2- | | |
| | methyl-pyrrolidine | | |
| 254 | 1-[6-(2,3-Dihydro-benzo[1,4]dioxin-6- | 350.105 | 349.2 |
| | yl)-1,2,3,4-tetrahydro-naphthalen-2- | | |
| | yl]-2-methyl-pyrrolidine | | |
| 255 | 3,5-Dimethyl-1-{3-[6-(2-methyl- | 193.569 | 385.25 |
| | pyrrolidin-1-yl)-5,6,7,8-tetrahydro- | | |
| | naphthalen-2-yl]-phenyl}-1H- | | |
| | pyrazole | | |
| 256 | 5-[6-(2-Methyl-pyrrolidin-1-yl)- | 294.106 | 293.19 |
| | 5,6,7,8-tetrahydro-naphthalen-2-yl]- | | |
| | pyrimidine | | |
| 257 | 1-[6-(4-Methoxy-phenyl)-1,2,3,4- | 322.122 | 321.21 |
| | tetrahydro-naphthalen-2-yl]-2- | | |
| | methyl-pyrrolidine | | |
| 258 | 1-[6-(4-Methoxy-2,6-dimethyl- | 350.134 | 349.24 |
| | phenyl)-1,2,3,4-tetrahydro- | | |
| | naphthalen-2-yl]-2-methyl-pyrrolidine | | |
| 259 | 1-[6-(2,3-Dihydro-benzofuran-5-yl)- | 334.111 | 333.21 |
| | 1,2,3,4-tetrahydro-naphthalen-2-yl]- | | |
| | 2-methyl-pyrrolidine | | |
| 260 | 1-[6-(4-Ethylsulfanyl-phenyl)-1,2,3,4- | 352.101 | 351.2 |
| | tetrahydro-naphthalen-2-yl]-2- | | |
| | methyl-pyrrolidine | | |
| 261 | 1-{6-[4-(1-Methoxy-ethyl)-phenyl]- | 350.136 | 349.24 |
| | 1,2,3,4-tetrahydro-naphthalen-2-yl}- | | |
| | 2-methyl-pyrrolidine | | |
| 262 | 1-[6-(2-Chloro-5-fluoro-phenyl)- | 344.043 | 343.15 |
| | 1,2,3,4-tetrahydro-naphthalen-2-yl]- | | |
| | 2-methyl-pyrrolidine | | |
| 263 | 1-Benzenesulfonyl-3-[6-(2-methyl- | 471.077 | 470.2 |
| | pyrrolidin-1-yl)-5,6,7,8-tetrahydro- | | |
| | naphthalen-2-yl]-1H-indole | | |
| 264 | 1-[6-(5-Chloro-2-fluoro-phenyl)- | 344.049 | 343.15 |
| | 1,2,3,4-tetrahydro-naphthalen-2-yl]- | | |
| | 2-methyl-pyrrolidine | | |
| 265 | 2-Isopropyl-5-[6-(2-methyl-pyrrolidin- | 188.087 | 374.27 |
| | 1-yl)-5,6,7,8-tetrahydro-naphthalen- | | |
| | 2-yl]-2,3-dihydro-1H-isoindole | | |
| 266 | 2-[Ethyl-(6-pyridin-3-yl-1,2,3,4- | 296.189 | 296.19 |
| | tetrahydro-naphthalen-2-yl)-amino]- | | |
| | ethanol | | |
| 267 | [1-(6-Pyridin-3-yl-1,2,3,4-tetrahydro- | 322.205 | 322.2 |
| | naphthalen-2-yl)-piperidin-3-yl]- | | |
| | methanol | | |
| 268 | 3-(6-Azetidin-1-yl-5,6,7,8-tetrahydro- | 264.163 | 264.16 |
| | naphthalen-2-yl)-pyridine | | |
| 269 | 4-(6-Azetidin-1-yl-5,6,7,8-tetrahydro- | 307 | 306.17 |
| | naphthalen-2-yl)-benzamide | | |
| 270 | 1-[6-(6-Methoxy-2-methyl-pyridin-3- | 394.67 | 393.24 |
| | yl)-1,2,3,4-tetrahydro-naphthalen-2- | | |
| | yl]-pyrrolidine-2-carboxylic acid | | |
| | dimethylamide | | |
| 271 | 1-[6-(4-Methanesulfonyl-phenyl)- | 427.62 | 426.2 |
| | 1,2,3,4-tetrahydro-naphthalen-2-yl]- | | |
| | pyrrolidine-2-carboxylic acid | | |
| | dimethylamide | | |
| 272 | 1-[6-(4-Methanesulfonyl-phenyl)- | 342.59 | 341.14 |
| | 1,2,3,4-tetrahydro-naphthalen-2-yl]- | | |
| | azetidine | | |
| 273 | 1-[6-(4-Ethanesulfonyl-phenyl)- | 441.64 | 440.21 |
| | 1,2,3,4-tetrahydro-naphthalen-2-yl]- | | |
| | pyrrolidine-2-carboxylic acid | | |
| | dimethylamide | | |
| 274 | 1-[6-(4-Methanesulfonyl-phenyl)- | 400.59 | 399.19 |
| | 1,2,3,4-tetrahydro-naphthalen-2-yl]- | | |
| | 2-methoxymethyl-pyrrolidine | | |
| 275 | 1-[6-(4-Ethanesulfonyl-phenyl)- | 414.65 | 413.2 |
| | 1,2,3,4-tetrahydro-naphthalen-2-yl]- | | |
| | 2-methoxymethyl-pyrrolidine | | |
| 276 | 1-[6-(4-Ethanesulfonyl-phenyl)- | 356.58 | 355.16 |
| | 1,2,3,4-tetrahydro-naphthalen-2-yl]- | | |
| | azetidine | | |
| 277 | 1-[6-(6-Methoxy-pyridin-3-yl)-1,2,3,4- | 380.67 | 379.23 |
| | tetrahydro-naphthalen-2-yl]- | | |
| | pyrrolidine-2-carboxylic acid | | |
| | dimethylamide | | |
| 278 | 1-[6-(3-Methanesulfonyl-phenyl)- | 400.2 | 399.19 |
| | 1,2,3,4-tetrahydro-naphthalen-2-yl]- | | |
| | 2-methoxymethyl-pyrrolidine | | |
| 279 | 2-Methoxy-5-[6-(2-methoxymethyl- | 353.68 | 352.22 |
| | pyrrolidin-1-yl)-5,6,7,8-tetrahydro- | | |
| | naphthalen-2-yl]-pyridine | | |
| 280 | 3-[6-(2-Methoxymethyl-pyrrolidin-1- | 393.67 | 392.25 |
| | yl)-5,6,7,8-tetrahydro-naphthalen-2- | | |
| | yl]-N,N-dimethyl-benzamide | | |
| 281 | 1-[6-(3-Methanesulfonyl-phenyl)- | 342.2 | 341.14 |
| | 1,2,3,4-tetrahydro-naphthalen-2-yl]- | | |
| | azetidine | | |
| 282 | 4-[6-(2-Methoxymethyl-pyrrolidin-1- | 393.67 | 392.25 |
| | yl)-5,6,7,8-tetrahydro-naphthalen-2- | | |
| | yl]-N,N-dimethyl-benzamide | | |
| 283 | 3-[6-(2-Methoxymethyl-pyrrolidin-1- | 365.62 | 364.22 |
| | yl)-5,6,7,8-tetrahydro-naphthalen-2- | | |
| | yl]-benzamide | | |
| 284 | 4-[6-(2-Methoxymethyl-pyrrolidin-1- | 379.67 | 378.23 |
| | yl)-5,6,7,8-tetrahydro-naphthalen-2- | | |
| | yl]-N-methyl-benzamide | | |
| 285 | 1-[6-(4-Dimethylcarbamoyl-phenyl)- | 420.3 | 419.26 |
| | 1,2,3,4-tetrahydro-naphthalen-2-yl]- | | |
| | pyrrolidine-2-carboxylic acid | | |
| | dimethylamide | | |
| 286 | 3-[6-(2-Phenyl-pyrrolidin-1-yl)- | 397.62 | 396.22 |
| | 5,6,7,8-tetrahydro-naphthalen-2-yl]- | | |
| | benzamide | | |
| 287 | 3-(6-Azetidin-1-yl-5,6,7,8-tetrahydro- | 307.57 | 306.17 |
| | naphthalen-2-yl)-benzamide | | |
| 288 | 1-Isopropyl-4-[6-(6-methoxy-2- | 380.71 | 379.26 |
| | methyl-pyridin-3-yl)-1,2,3,4- | | |
| | tetrahydro-naphthalen-2-yl]- | | |
| | piperazine | | |
| 289 | 1-[6-(4-Methanesulfonyl-phenyl)- | 370.61 | 369.18 |
| | 1,2,3,4-tetrahydro-naphthalen-2-yl]- | | |
| | piperidine | | |
| 290 | 1-Isopropyl-4-[6-(4-methanesulfonyl- | 413.66 | 412.22 |
| | phenyl)-1,2,3,4-tetrahydro- | | |
| | naphthalen-2-yl]-piperazine | | |
| 291 | 1-[6-(6-Methoxy-2-methyl-pyridin-3- | 353.52 | 352.22 |
| | yl)-1,2,3,4-tetrahydro-naphthalen-2- | | |
| | yl]-piperidin-3-ol | | |
| 292 | 1-[6-(4-Ethanesulfonyl-phenyl)- | 427.68 | 426.23 |
| | 1,2,3,4-tetrahydro-naphthalen-2-yl]- | | |
| | 4-isopropyl-piperazine | | |
| 293 | 1-[6-(4-Methanesulfonyl-phenyl)- | 386.65 | 385.17 |
| | 1,2,3,4-tetrahydro-naphthalen-2-yl]- | | |
| | piperidin-3-ol | | |
| 294 | 1-[6-(4-Ethanesulfonyl-phenyl)- | 384.63 | 383.19 |
| | 1,2,34-tetrahydro-naphthalen-2-yl]- | | |
| | piperidine | | |
| 295 | 4-[6-(6-Methoxy-2-methyl-pyridin-3- | 355.59 | 354.18 |
| | yl)-1,2,3,4-tetrahydro-naphthalen-2- | | |
| | yl]-thiomorpholine | | |
| 296 | [6-(4-Ethanesulfonyl-phenyl)-1,2,3,4- | 372.62 | 371.19 |
| | tetrahydro-naphthalen-2-yl]-diethyl- | | |
| | amine | | |
| 297 | 1-[6-(4-Ethanesulfonyl-phenyl)- | 400.60 | 399.19 |
| | 1,2,3,4-tetrahydro-naphthalen-2-yl]- | | |
| | piperidin-3-ol | | |
| 298 | 6-Methoxy-2-methyl-3-(6-piperidin-1- | 337.64 | 336.22 |
| | yl-5,6,7,8-tetrahydro-naphthalen-2- | | |
| | yl)-pyridine | | |
| 299 | Diethyl-[6-(4-methanesulfonyl- | 358.62 | 357.18 |
| | phenyl)-1,2,3,4-tetrahydro- | | |
| | naphthalen-2-yl]-amine | | |
| 300 | 4-[6-(4-Methanesulfonyl-phenyl)- | 386.61 | 385.17 |
| | 1,2,3,4-tetrahydro-naphthalen-2-yl]- | | |
| | 3-methyl-morpholine | | |
| 301 | Diethyl-(6-pyrimidin-5-yl-1,2,3,4- | 282.54 | 281.19 |
| | tetrahydro-naphthalen-2-yl)-amine | | |
| 302 | 4-[6-(4-Ethanesulfonyl-phenyl)- | 402.57 | 401.15 |
| | 1,2,3,4-tetrahydro-naphthalen-2-yl]- | | |
| | thiomorpholine | | |
| 303 | 4-[6-(6-Methoxy-2-methyl-pyridin-3- | 353.64 | 352.22 |
| | yl)-1,2,3,4-tetrahydro-naphthalen-2- | | |
| | yl]-3-methyl-morpholine | | |
| 304 | 4-[6-(4-Ethanesulfonyl-phenyl)- | 400.65 | 399.19 |
| | 1,2,3,4-tetrahydro-naphthalen-2-yl]- | | |
| | 3-methyl-morpholine | | |
| 305 | 1-Isopropyl-4-[6-(6-methoxy-pyridin- | 366.74 | 365.25 |
| | 3-yl)-1,2,3,4-tetrahydro-naphthalen- | | |
| | 2-yl]-piperazine | | |
| 306 | 4-[6-(6-Methoxy-pyridin-3-yl)-1,2,3,4- | 341.60 | 340.16 |
| | tetrahydro-naphthalen-2-yl]- | | |
| | thiomorpholine | | |
| 307 | 5-[6-(4-Isopropyl-piperazin-1-yl)- | 337.64 | 336.23 |
| | 5,6,7,8-tetrahydro-naphthalen-2-yl]- | | |
| | pyrimidine | | |
| 308 | 4-[6-(4-Isopropyl-piperazin-1-yl)- | 378.73 | 377.25 |
| | 5,6,7,8-tetrahydro-naphthalen-2-yl]- | | |
| | benzamide | | |
| 309 | 1-(6-Pyrimidin-5-yl-1,2,3,4- | 310.52 | 309.18 |
| | tetrahydro-naphthalen-2-yl)- | | |
| | piperidin-3-ol | | |
| 310 | 1-Isopropyl-4-[6-(3-methanesulfonyl- | 413.66 | 412.22 |
| | phenyl)-1,2,3,4-tetrahydro- | | |
| | naphthalen-2-yl]-piperazine | | |
| 311 | 1-[6-(6-Methoxy-pyridin-3-yl)-1,2,3,4- | 339.62 | 338.2 |
| | tetrahydro-naphthalen-2-yl]-piperidin- | | |
| | 3-ol | | |
| 312 | 5-(6-Piperidin-1-yl-5,6,7,8- | 294.62 | 293.19 |
| | tetrahydro-naphthalen-2-yl)- | | |
| | pyrimidine | | |
| 313 | 4-[6-(3-Hydroxy-piperidin-1-yl)- | 351.64 | 350.2 |
| | 5,6,7,8-tetrahydro-naphthalen-2-yl]- | | |
| | benzamide | | |
| 314 | 2-Methoxy-5-(6-piperidin-1-yl- | 323.66 | 322.2 |
| | 5,6,7,8-tetrahydro-naphthalen-2-yl)- | | |
| | pyridine | | |
| 315 | 1-[6-(3-Methanesulfonyl-phenyl)- | 386.61 | 385.17 |
| | 1,2,3,4-tetrahydro-naphthalen-2-yl]- | | |
| | piperidin-3-ol | | |
| 316 | 4-(6-Thiomorpholin-4-yl-5,6,7,8- | 3.53.57 | 352.16 |
| | tetrahydro-naphthalen-2-yl)- | | |
| | benzamide | | |
| 317 | 3-[6-(4-Isopropyl-piperazin-1-yl)- | 406.66 | 405.28 |
| | 5,6,7,8-tetrahydro-naphthalen-2-yl]- | | |
| | N,N-dimethyl-benzamide | | |
| 318 | 3-[6-(3-Hydroxy-piperidin-1-yl)- | 379.68 | 378.23 |
| | 5,6,7,8-tetrahydro-naphthalen-2-yl]- | | |
| | N,N-dimethyl-benzamide | | |
| 319 | 3-[6-(2-Isopropyl-pyrrolidin-1-yl)- | 365.39 | 364.25 |
| | 5,6,7,8-tetrahydro-naphthalen-2-yl]- | | |
| | 6-methoxy-2-methyl-pyridine | | |
| 320 | 1-[6-(4-Ethanesulfonyl-phenyl)- | ND | 383.19 |
| | 1,2,3,4-tetrahydro-naphthalen-2-yl]- | | |
| | 2-methyl-pyrrolidine | | |
| 321 | 2-[6-(4-Ethanesulfonyl-phenyl)- | 418.26 | 417.18 |
| | 1,2,3,4-tetrahydro-naphthalen-2-yl]- | | |
| | 2,3-dihydro-1H-isoindole | | |
| 322 | 2-[6-(6-Methoxy-2-methyl-pyridin-3- | 371.32 | 370.2 |
| | yl)-1,2,3,4-tetrahydro-naphthalen-2- | | |
| | yl]-2,3-dihydro-1H-isoindole | | |
| 323 | 1-[6-(4-Ethanesulfonyl-phenyl)- | 412.32 | 411.22 |
| | 1,2,3,4-tetrahydro-naphthalen-2-yl]- | | |
| | 2-isopropyl-pyrrolidine | | |
| 324 | 5-[6-(2-Isopropyl-pyrrolidin-1-yl)- | 322.36 | 321.22 |
| | 5,6,7,8-tetrahydro-naphthalen-2-yl]- | | |
| | pyrimidine | | |
| 325 | 4-[6-(4-Methanesulfonyl-phenyl)- | 372.28 | 371.16 |
| | 1,2,3,4-tetrahydro-naphthalen-2-yl]- | | |
| | morpholine | | |
| 326 | 4-[6-(4-Ethanesulfonyl-phenyl)- | 386.28 | 385.17 |
| | 1,2,3,4-tetrahydro-naphthalen-2-yl]- | | |
| | morpholine | | |
| 327 | 4-[6-(6-Methoxy-2-methyl-pyridin-3- | 339.34 | 338.2 |
| | yl)-1,2,3,4-tetrahydro-naphthalen-2- | | |
| | yl]-morpholine | | |
| 328 | 4-(6-Pyrimidin-5-yl-1,2,3,4- | 296.31 | 295.17 |
| | tetrahydro-naphthalen-2-yl)- | | |
| | morpholine | | |
| 329 | 3-[6-(2-Isopropyl-pyrrolidin-1-yl)- | 391.37 | 390.27 |
| | 5,6,7,8-tetrahydro-naphthalen-2-yl]- | | |
| | N,N-dimethyl-benzamide | | |
| 330 | 1-[6-(4-Methanesulfonyl-phenyl)- | 372.27 | 371.16 |
| | 1,2,3,4-tetrahydro-naphthalen-2-yl]- | | |
| | pyrrolidin-3-ol | | |
| 331 | 4-[6-(1,3-Dihydro-isoindol-2-yl)- | 397.35 | 396.22 |
| | 5,6,7,8-tetrahydro-naphthalen-2-yl]- | | |
| | N,N-dimethyl-benzamide | | |
| 332 | 2-Methoxy-5-[6-(3-phenyl-pyrrolidin- | 385.33 | 384.22 |
| | 1-yl)-5,6,7,8-tetrahydro-naphthalen- | | |
| | 2-yl]-pyridine | | |
| 333 | 3-[6-(1,3-Dihydro-isoindol-2-yl)- | 397.35 | 396.22 |
| | 5,6,7,8-tetrahydro-naphthalen-2-yl]- | | |
| | N,N-dimethyl-benzamide | | |
| 334 | 4-[6-(6-Methoxy-pyridin-3-yl)-1,2,3,4- | 325.32 | 324.18 |
| | tetrahydro-naphthalen-2-yl]- | | |
| | morpholine | | |
| 335 | 3-[6-(3-Phenyl-pyrrolidin-1-yl)- | 397.34 | 396.22 |
| | 5,6,7,8-tetrahydro-naphthalen-2-yl]- | | |
| | benzamide | | |
| 336 | 5-[6-(2-Isopropyl-pyrrolidin-1-yl)- | 351.37 | 350.24 |
| | 5,6,7,8-tetrahydro-naphthalen-2-yl]- | | |
| | 2-methoxy-pyridine | | |
| 337 | 4-[6-(Benzyl-methyl-amino)-5,6,7,8- | 399.35 | 398.24 |
| | tetrahydro-naphthalen-2-yl]-N,N- | | |
| | dimethyl-benzamide | | |
| 338 | 4-[6-(3-Methanesulfonyl-phenyl)- | 372.27 | 371.16 |
| | 1,2,3,4-tetrahydro-naphthalen-2-yl]- | | |
| | morpholine | | |
| 339 | 1-[6-(3-Methanesulfonyl-phenyl)- | 432.28 | 431.19 |
| | 1,2,3,4-tetrahydro-naphthalen-2-yl]- | | |
| | 3-phenyl-pyrrolidine | | |

The composition of the present invention may be a composition comprising a compound of formula I and optionally a pharmaceutically acceptable carrier. The composition of the present invention may also be a composition comprising a compound of formula I, a histamine H₁ antagonist and optionally a pharmaceutically acceptable carrier. The composition of the present invention may also be a composition comprising a compound of formula I, a neurotransmitter re-uptake blocker and optionally a pharmaceutically acceptable carrier.

The composition of the present invention may be formulated in a conventional manner using one or more pharmaceutically acceptable carriers. The composition may be formulated for oral, buccal, intranasal, parenteral (e.g., intravenous, intramuscular, intraperitoneal, or subcutaneous or through an implant) nasal, vaginal, sublingual, rectal or topical administration or in a form suitable for administration by inhalation or insufflation.

Pharmaceutically acceptable salts of compounds of formula I may be prepared by one or more of three methods: (i) by reacting the compound of formula I with the desired acid or base; (ii) by removing an acid- or base-labile protecting group from a suitable precursor of the compound of formula I or by ring-opening a suitable cyclic precursor, for example, a lactone or lactam, using the desired acid or base; or (iii) by converting one salt of the compound of formula I to another by reaction with an appropriate acid or base or by means of a suitable ion exchange column.

All three reactions are typically carried out in solution. The resulting salt may precipitate out and be collected by filtration or may be recovered by evaporation of the solvent. The degree of ionisation in the resulting salt may vary from completely ionised to almost non-ionised.

Also included within the scope of the invention are metabolites of compounds of formula I, that is, compounds formed in vivo upon administration of the drug. Some examples of metabolites in accordance with the invention include: (i) where the compound of formula (I) contains a methyl group, an hydroxymethyl derivative thereof (—CH₃—CH₂OH); (ii) where the compound of formula (I) contains an alkoxy group, an hydroxy derivative thereof (—OR→—OH); (iii) where the compound of formula (I) contains a tertiary amino group, a secondary amino derivative thereof (—NR^{a}R^{b}→—NHR^{a} or —NHR^{b}); (iv) where the compound of formula (I) contains a secondary amino group, a primary derivative thereof (—NHR^{a}→—NH₂); (v) where the compound of formula (I) contains an amide group, a carboxylic acid derivative thereof (—CONR^{c}R^{d}→—COOH).

Isotopically labeled compounds of formula I of this invention can generally be prepared by carrying out the procedures disclosed in the preceeding Schemes and/or in the Examples and Preparations, by substituting a readily available isotopically labeled reagent for a non-isotopically labeled reagent.

For oral administration, the pharmaceutical composition may take the form of, for example, tablets or capsules prepared by conventional means with pharmaceutically acceptable excipients such as binding agents such as pregelatinized maize starch, polyvinylpyrrolidone or hydroxypropyl methylcellulose; fillers such as lactose, microcrystalline cellulose or calcium phosphate; lubricants such as magnesium stearate, talc or silica; disintegrants such as potato starch or sodium starch glycolate; or wetting agents such as sodium lauryl sulphate. The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents such as sorbitol syrup, methyl cellulose or hydrogenated edible fats; emulsifying agents such as lecithin or acacia, non-aqueous vehicles such as almond oil, oily esters or ethyl alcohol; and preservatives such as methyl or propyl p-hydroxybenzoates or sorbic acid.

For buccal administration, the composition may take the form of tablets or lozenges formulated in conventional manner.

The composition of the invention may be formulated for parenteral administration by injection, including using conventional catheterization techniques or infusion. Formulations for injection may be presented in unit dosage form, for example, in ampoules or in multi-dose containers, with an added preservative. The composition may take such forms as suspensions, solutions or emulsions in oily or aqueous vehicles, and may contain formulating agents such as suspending, stabilizing and/or dispersing agents. Alternatively, the active ingredient or ingredients in a composition may be in powder form for reconstitution with a suitable vehicle, for example, sterile pyrogen-free water, before use. The term “active ingredient” as used herein refers to a compound of the formula I, a histamine H₁ antagonist, or a neurotransmitter re-uptake blocker.

The composition of the invention may also be formulated in a rectal composition such as suppositories or retention enemas, for example, containing conventional suppository bases such as cocoa butter or other glycerides. A composition for vaginal administration is preferably a suppository that may contain, in addition to the active ingredient or ingredients, excipients such as cocoa butter or a suppository wax. A composition for nasal or sublingual administration is also prepared with standard excipients well known in the art.

For intranasal administration or administration by inhalation, the composition may be conveniently delivered in the form of a solution or suspension from a pump spray container that is squeezed or pumped by the patient or as an aerosol spray presentation from a pressurized container or a nebulizer, with the use of a suitable propellant, for example, dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active ingredient or ingredients. Capsules and cartridges, made, for example, from gelatin, for use in an inhaler or insufflator may be formulated containing a powder mix of an active ingredient or ingredients and a suitable powder base such as lactose or starch. The active ingredient or ingredients in the composition may range in size from nanoparticles to microparticles.

An exemplary dose of the composition of the invention comprising a compound of formula I for oral, parenteral or buccal administration to the average adult human for the treatment of the conditions referred to herein is about 0.01 to about 1000 mg of the compound of formula I per unit dose which could be administered, for example, 1 to 3 times per day.

An exemplary dose of the composition of the invention comprising a compound of formula I and a histamine H₁ antagonist or a neurotransmitter re-uptake blocker for oral, parenteral or buccal administration to the average adult human for the treatment of the conditions referred to herein is about 0.01 to about 500 mg of the compound of formula I and of about 0.01 mg to about 500 mg of the histamine H₁ antagonist or the neurotransmitter re-uptake blocker per unit dose which could be administered, for example, 1 to 3 times per day.

Aerosol formulations for treatment of the conditions referred to herein in the average adult human are preferably arranged so that each metered dose or “puff” of aerosol contains about 20 μg to about 1000 μg of the compound of formula I. The overall daily dose with an aerosol will be within the range about 100 μg to about 10 mg. Administration may be several times daily, for example 2, 3, 4 or 8 times, giving for example, 1, 2 or 3 doses each time. Aerosol formulations containing a compound of formula I and a histamine H₁ antagonist or a neurotransmitter re-uptake blocker are preferably arranged so that each metered dose or “puff” of aerosol contains about 100 μg to about 10,000 μg of the compound of formula I and about 100 μg to about 30,000 μg of the histamine H₁ antagonist or the neurotransmitter re-uptake blocker. Administration may be several times daily, for example 1, 3, 4 or 8 times, giving for example, 1, 2 or 3 doses each time. The composition of the invention comprising a compound of formula I and a histamine H₁ antagonist or a neurotransmitter re-uptake blocker may optionally contain a pharmaceutically acceptable carrier and may be administered in both single and multiple dosages as a variety of different dosage forms, such as tablets, capsules, lozenges, troches, hard candies, powders, sprays, aqueous suspension, injectable solutions, elixirs, syrups; and the like. The pharmaceutically acceptable carriers include solid diluents or fillers, sterile aqueous media and various non-toxic organic solvents, etc. Oral pharmaceutical formulations can be suitably sweetened and/or flavored by means of various agents of the type commonly employed for such purposes. In general, the compound of formula I is present in such dosage forms at concentration levels ranging from about 0.1% to about 99.9% by weight of the total composition, i.e., in amounts which are sufficient to provide the desired unit dosage, and the histamine H₁ antagonist or the neurotransmitter re-uptake blocker is present in such dosage forms at concentration levels ranging from about 0.1% to about 99.9% by weight of the total composition, i.e., in amounts which are sufficient to provide the desired unit dosage.

The compound of formula I and the histamine H₁ antagonist may be administered together or separately. When administered separately, the compound of formula I and the histamine H₁ antagonist may be administered in either order, provided that after administration of the first of the two active ingredients, the second active ingredient is administered within 24 hours or less, preferably 12 hours or less.

The compound of formula I and the neurotransmitter re-uptake blocker may be administered together or separately. When administered separately, the compound of formula I and the neurotransmitter re-uptake blocker may be administered in either order, provided that after administration of the first of the two active ingredients, the second active ingredient is administered within 24 hours or less, preferably 12 hours or less.

A preferred dose ratio of compound of formula I to the histamine H₁ antagonist or to the neurotransmitter re-uptake blocker for oral, parenteral or buccal administration to the average adult human for the treatment of the conditions referred to herein is from about 0.001 to about 1000, preferably from about 0.01 to about 100.

The composition may be homogeneous, wherein by homogeneous it is meant that the active ingredient or ingredients are dispersed evenly throughout the composition so that the composition may be readily subdivided into equally effective unit dosage forms such as tablets, pills and capsules. This solid composition is then subdivided into unit dosage forms of the type described herein containing from about 0.1 to about 1000 mg of the active ingredient or ingredients. Typical unit dosage forms contain from about 1 to about 300 mg, for example about 1, 2, 5, 10, 25, 50 or 100 mg, of the active ingredient or ingredients. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The dosage of the active ingredient or ingredients in the composition and methods of this invention may be varied; however, it is necessary that the amount of the active ingredient or ingredients in such a composition be such that a suitable dosage form is obtained. The selected dosage depends upon the desired therapeutic effect, on the route of administration, the particular compounds administered, the duration of the treatment, and other factors. All dosage ranges and dosage levels mentioned herein refer to each active ingredient present in the pharmaceutical composition of the present invention, as well as those used in the methods of the present invention. Generally, dosage levels of between about 0.01 and about 100 mg/kg of body weight daily are administered to humans and other mammals. A preferred dosage range in humans is about 0.1 to about 50 mg/kg of body weight daily which can be administered as a single dose or divided into multiple doses. A preferred dosage range in mammals other than humans is about 0.01 to about 10.0 mg/kg of body weight daily which can be administered as a single dose or divided into multiple doses. A more preferred dosage range in mammals other than humans is about 0.1 to about 5.0 mg/kg of body weight daily which can be administered as a single dose or divided into multiple doses.

The pharmaceutical composition comprising the compound of formula I and the histamine H₁ antagonist or the neurotransmitter re-uptake blocker may be administered at dosages of a therapeutically effective amount of the compound of formula I and of the second active ingredient in single or divided doses.

The specific therapeutically effective dose level for any particular patient will depend upon a variety of factors including the disorder being treated and the severity of the disorder; activity of the specific compound employed; the specific composition employed; the age. However, some variation in dosage will necessarily occur depending upon the condition of the subject being treated. The person responsible for administration will, in any event, determine the appropriate dose for the individual subject.

The dosage amounts set forth in this description and in the appended claims may be used, for example, for an average human subject having a weight of about 65 kg to about 70 kg. The skilled practitioner will readily be able to determine any variation in the dosage amount that may be required for a subject whose weight falls outside the about 65 kg to about 70 kg range based upon the medical history of the subject. The pharmaceutical combinations may be administered on a regimen of up to 6 times per day, preferably 1 to 3 times per day, such as 2 times per day or once daily.

### Determination of Biological Activity

The in vitro affinity of the compounds in the present invention at the rat or human histamine H3 receptors can be determined according to the following procedure. Frozen rat frontal brain or frozen human post-mortem frontal brain is homogenized in 20 volumes of cold 50 mM Tris HCl containing 2 mM MgCl₂ (pH to 7.4 at 4° C.). The homogenate is then centrifuged at 45,000 G for 10 minutes. The supernatant is decanted and the membrane pellet resuspended by Polytron in cold 50 mM Tris HCl containing 2 mM MgCl₂ (pH to 7.4 at 4° C.) and centrifuged again. The final pellet is resuspended in 50 mM Tris HCl containing 2 mM MgCl₂ (pH to 7.4 at 25° C.) at a concentration of 12 mg/mL. Dilutions of compounds are made in 10% DMSO/50 mM Tris buffer (pH 7.4) (at 10× final concentration, so that the final DMSO concentration is 1%). Incubations are initiated by the addition of membranes (200 microliters) to 96 well V-bottom polypropylene plates containing 25 microliters of drug dilutions and 25 microliters of radioligand (1 nM final concentration 3H-N-methyl-histamine). After a 1 hour incubation, assay samples are rapidly filtered through Whatman GF/B filters and rinsed with ice-cold 50 mM Tris buffer (pH 7.4) using a Skatron cell harvester. Radioactivity is quantified using a BetaPlate scintillation counter. The percent inhibition of specific binding can then be calculated,

A person of ordinary skill in the art could adapt the above procedure to other assays.

We claim:

1. A compound according to formula I, 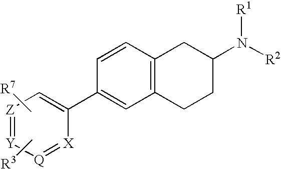
or a pharmaceutically acceptable salt thereof, wherein
Z, Y, Q, X are independently nitrogen or carbon;
R¹ and R² are independently hydrogen, (C₁-C₈)alkyl optionally substituted with 1 to 4 halogens, or (C₃-C₇)cycloalkyl-(C₀-C₄)alkyl, wherein each (C₀-C₄) is optionally substituted with one to four (C₁-C₄)alkyl;
or optionally R¹ and R², together with the nitrogen to which they are attached, form a 4 to 7-membered heterocycloalkyl ring, wherein one of the carbons of said heterocycloalkyl ring that is separated by at least two atoms from said nitrogen in said heterocycloalkyl ring is optionally replaced by O, S, NR⁶, or C═O, wherein R⁶ is hydrogen, (C₁-C₃)alkyl, or —C(═O)(C₁-C₃)alkyl; and wherein said heterocycloalkyl ring is optionally substituted with halo, one or two (C₁-C₄)alkyl, phenyl, (C₁-C₈)alkyl optionally substituted with 1 to 4 halogens, or (C₃-C₇)cycloalkyl-(C₀-C₄)alkyl, and wherein each (C₀-C₄)alkyl is optionally substituted with one to four (C₁-C₄)alkyl;
R₃ is hydrogen, (C₁-C₆)alkyl, (C₁-C₆)alkoxy, halo, 5 to 6-membered aryl, 5 to 6 membered heteroaryl, hydroxyl, methylene hydroxyl, —(C═O)NR₄R₅, and S(O)ₚ(C₁-C₄)alkyl, where p is 1 or 2;
wherein R⁴ and R⁵ are independently selected from the group consisting of hydrogen;
(C₁-C₈)alkyl optionally substituted with 1 to 4 halogens;
(C₁-C₄)alkyl group optionally substituted with a substituent selected from the group consisting of OH, 1 to 4 (C₁-C₄)alkyl, (C₃-C₇)cycloalkyl, (C₁-C₄)dialkylamino, (C₆-C₁₄)aryl optionally substituted with a halogen and optionally substituted with (C₆-C₁₀)aryloxy optionally substituted with 1 to 2 halogens, and 5 to 10-membered heteroaryl optionally substituted with a (C₆-C₁₀)aryl group and optionally substituted with 1 to 3 (C₁-C₄)alkyl groups;
(C₃-C₇)cycloalkyl;
(C₆-C₁₄)aryl;
-(C₂-C₃)alkyl-O—(C₁-C₃)alkyl optionally substituted with (C₁-C₃)alkyl;
-(C₁-C₃)alkyl-C(═O)O—(C₁-C₃)alkyl;
3-8-membered heterocycloalkyl optionally substituted with one or more (C₁-C₄)alkyl-carbonyl groups;
(C₈-C₁₀)arylsulfonyl optionally substituted with one or more (C₁-C₂)alkyl;
5-10-membered heteroaryl; and
(C₆-C₄)aryl-(C₀-C₄)alkylene-O—(C₀-C₄)alkyl, wherein each (C₀-C₄)alkyl and each (C₀-C₄)alkylene is optionally substituted with 1 to 4 (C₁-C₄ alkyl);
or optionally R⁴ and R⁵, together with the nitrogen to which they attached, form a 4 to 6-membered heterocyclic ring, wherein one of the carbons of said heterocyclic ring that is separated by at least two atoms from said nitrogen in said heterocyclic ring is optionally replaced by O or NR⁶, wherein R⁸ is hydrogen, (C₁-C₃)alkyl, or —C(═O)(C₁-C₃)alkyl; and wherein said heterocyclic ring is optionally substituted with halo, (C₁-C₃)alkyl, or hydroxyl;
R⁷ is hydrogen;
or optionally R³ and R⁷ together with two adjacent atoms in the ring comprising Z, Y, Q and X to which they are attached, form a 5 or 6-membered heterocyclic ring, wherein one of the carbons of said heterocyclic ring that is separated by at least two atoms from said nitrogen in said heterocyclic ring is optionally replaced by O or NR⁶; wherein R⁸ is hydrogen or (C₁-C₃)alkyl.

2. A compound of formula I, according to claim 1, wherein Y and X are carbon; Q and Z are carbon or nitrogen; R⁷ is hydrogen; R¹ and R² together form a 5-membered heterocycloalkyl ring, optionally substituted with (C₁-C₄)alkyl; and R³ is selected from the group consisting of methoxy,
—(C═O)NR₄R⁵, and S(O)ₚ(C₁-C₄)alkyl; wherein R⁴ and R⁵ are independently hydrogen or (C₁-C₄)alkyl; and wherein p is 1 or 2.

3. A compound of formula I, according to claim 1, wherein Z, Y, X, and Q are carbon;
R¹ and R² together with the nitrogen to which they are attached form a 5-membered heterocycloalkyl ring optionally substituted with methyl;
R^{a7} is hydrogen;
R³ is —(C═O)NR₄R₅;
wherein R⁴ and R⁵ are independently selected from the group consisting of hydrogen;
(C₁-C₈)alkyl optionally substituted with 1 to 4 halogens;
(C₁-C₄)alkyl group optionally substituted with a substituent selected from the group consisting of OH, 1 to 4 (C₁-C₄)alkyl, (C₃-C₇)cycloalkyl, (C₁-C₄)dialkylamino, (C₆-C₁₄)aryl optionally substituted with a halogen and optionally substituted with (C₆-C₁₀)aryloxy optionally substituted with 1 to 2 halogens, and 5 to 10-membered heteroaryl optionally substituted with a (C₆-C₁₀)aryl group and optionally substituted with 1 to 3 (C₁-C₄)alkyl groups;
(C₃-C₇)cycloalkyl;
(C₆-C₁₄)aryl;
-(C₂-C₃)alkyl-O—(C₁-C₃)alkyl optionally substituted with (C₁-C₃)alkyl,
—(C₁-C₃)alkyl-C(═O)O—(C₁-C₃)alkyl;
3-8-membered heterocycloalkyl optionally substituted with one or more (C₁-C₄)alkyl-carbonyl groups;
(C₆-C₁₀)arylsulfonyl optionally substituted with one or more (C₁-C₂)alkyl;
5-10-membered heteroaryl; and
(C₆-C₁₄)aryl-(C₀-C₄)alkylene-O—(C₀₋₄)alkyl, wherein each (C₀-C₄)alkyl and each (C₀-C₄)alkylene is optionally substituted with 1 to 4 (C₁-C₄ alkyl);
or optionally R⁴ and R⁵, together with the nitrogen to which they attached, form a 4 to 6-membered heterocyclic ring, wherein one of the carbons of said heterocycloalkyl ring that is separated by at least two atoms from said nitrogen in said heterocycloalkyl ring is optionally replaced by O or NR⁸, wherein R⁸ is hydrogen or (C₁-C₃)alkyl; and wherein said heterocycloalkyl ring is optionally substituted with halo, (C₁-C₃)alkyl, or hydroxyl.

4. A compound of claim 3, wherein R⁴ and R⁵ are independently selected from the group consisting of hydrogen, (C₁-C₅)alkyl, (C₃-C₅)cycloalkyl.

5. A compound of claim 3, wherein R⁴ and R⁵, together with the nitrogen to which they are attached, form a 4 to 6-membered heterocycloalkyl ring, and wherein said heterocycloalkyl ring is optionally substituted with halo, hydroxy, or (C₁-C₅)alkyl.

6. A compound of formula I, according to claim 1, wherein X, Y, Z are carbon; Q is nitrogen;
R¹ and R² together with the nitrogen to which they are attached form a 5-membered heterocyclic ring optionally substituted with methyl; R³ is selected from the group consisting of hydrogen, methyl, ethyl, methoxy, and ethoxy; and R⁷ is hydrogen.

7. A compound of formula I, according to claim 1, wherein X is carbon; Z and Q are nitrogen; R³ is selected from the group consisting of hydrogen, methyl, ethyl, methoxy, and ethoxy; and R⁷ is hydrogen.

8. A compound of formula I, according to claim 1, wherein said compound has the following structure:

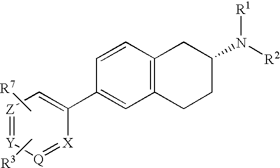

9. A compound of formula I, according to claim 1, wherein said compound has the following structure:

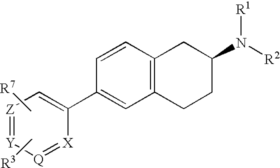

10. A compound of formula I, according to claim 1, selected from the group consisting of
N,N-dimethyl-3-(6-(pyrrolidin-1-yl)-5,6,7,8-tetrahydronaphthalen-2-yl)benzamide;
N-ethyl-N-methyl-3-(6-pyrrolidin-1-yl-5,6,7,8-tetrahydro-naphthalen-2-yl)-benzamide;
1-[6-(2,5-Dimethyl-phenyl)-1,2,3,4-tetrahydro-naphthalen-2-yl]-pyrrolidine;
2-(6-Pyrrolidin-1-yl-5,6,7,8-tetrahydro-naphthalen-2-yl)-benzaldehyde;
1-[6-(2-Phenoxy-phenyl)-1,2,3,4-tetrahydro-naphthalen-2-yl]-pyrrolidine:
1-[6-(4-Fluoro-3-methyl-phenyl)-1,2,3,4-tetrahydro-naphthalen-2-yl]-pyrrolidine;
1-[6-(4-Methoxy-3,5-dimethyl-phenyl)-1,2,3,4-tetrahydro-naphthalen-2-yl]-pyrrolidine;
1-[6-(3-Fluoro-4-methoxy-phenyl)-1,2,3,4-tetrahydro-naphthalen-2-yl]-pyrrolidine;
1-[6-(2,3,4-Trimethoxy-phenyl)-1,2,3,4-tetrahydro-naphthalen-2-yl]-pyrrolidine;
1-(6-Phenoxathiin-4-yl-1,2,3,4-tetrahydro-naphthalen-2-yl)-pyrrolidine;
[3-(6-Pyrrolidin-1-yl-5,6,7,8-tetrahydro-naphthalen-2-yl)-phenyl]-methanol;
3-[6-Pyrrolidin-1-yl-5,6,7,8-tetrahydro-naphthalen-2-yl]-benzonitrile;
1-(6-Phenyl-1,2,3,4-tetrahydro-naphthalen-2-yl)-pyrrolidine;
N,N-Diisopropyl-2-(6-pyrrolidin-1-yl-5,6,7,8-tetrahydro-naphthalen-2-yl)-benzamide;
1-[6-(4-Methylsulfanyl-phenyl)-1,2,3,4-tetrahydro-naphthalen-2-yl]-pyrrolidine;
1-(6-m-Tolyl-1,2,3,4-tetrahydro-naphthalen-2-yl)-pyrrolidine;
1-[6-(3-Chloro-4-fluoro-phenyl)-1,2,3,4-tetrahydro-naphthalen-2-yl]-pyrrolidine;
1-[6-(3-Nitro-phenyl)-1,2,3,4-tetrahydro-naphthalen-2-yl]-pyrrolidine;
1-[6-(3,5-Dimethyl-phenyl)-1,2,3,4-tetrahydro-naphthalen-2-yl]-pyrrolidine;
1-[6-(3-Fluoro-phenyl)-1,2,3,4-tetrahydro-naphthalen-2-yl]-pyrrolidine;
1-[6-(3,4-Dichloro-phenyl)-1,2,3,4-tetrahydro-naphthalen-2-yl]-pyrrolidine;
1-[6-(2-Methoxy-phenyl)-1,2,3,4-tetrahydronaphthalen-2-yl]-pyrrolidine;
1-[6-(3-Ethoxy-phenyl)-1,2,3,4-tetrahydro-naphthalen-2-yl]-pyrrolidine;
1-[6-(3,4-Difluoro-phenyl)-1,2,3,4-tetrahydro-naphthalen-2-yl]-pyrrolidine;
1-[6-(4-Fluoro-phenyl)-1,2,3,4-tetrahydro-naphthalen-2-yl]-pyrrolidine;
1-[6-(4-Trifluoromethoxy-phenyl)-1,2,3,4-tetrahydro-naphthalen-2-yl]-pyrrolidine;
1-(6-Benzo[1,3]dioxol-5-yl-1,2,3,4-tetrahydro-naphthalen-2-yl)-pyrrolidine;
1-(5,6,78-Tetrahydro-[2,2′]binaphthalenyl-6-yl)-pyrrolidine;
1-[6-(2-Chloro-phenyl)-1,2,3,4-tetrahydro-naphthalen-2-yl]-pyrrolidine;
1-[6-(4-Ethyl-phenyl)-1,2,3,4-tetrahydro-naphthalen-2-yl]-pyrrolidine;
1-[6-(2-Ethoxy-phenyl)-1,2,3,4-tetrahydro-naphthalen-2-yl]-pyrrolidine;
1-[4-(6-Pyrrolidin-1-yl-5,6,7,8-tetrahydro-naphthalen-2-yl)-phenyl]-ethanone;
1-[4-(6-Pyrrolidin-1-yl-5,6,7,8-tetrahydro-naphthalen-2-yl)-phenyl]-ethanone;
1-[6-(2,6-Dimethyl-phenyl)-1,2,3,4-tetrahydro-naphthalen-2-yl]-pyrrolidine;
1-[6-(4-Ethoxy-phenyl)-1,2,3,4-tetrahydro-naphthalen-2-yl]-pyrrolidine:
1-[3-(6-Pyrrolidin-1-yl-5,6,7,8-tetrahydro-naphthalen-2-yl)-phenyl]-ethanone;
1-[6-(2-Methoxy-5-methyl-phenyl)-1,2,3,4-tetrahydro-naphthalen-2-yl]-pyrrolidine;
Dimethyl-[4-(6-pyrrolidin-1-yl-5,6,7,8-tetrahydro-naphthalen-2-yl)-phenyl]-amine;
[4-(6-Pyrrolidin-1-yl-5,6,7,8-tetrahydro-naphthalen-2-yl)-phenyl]-methanol;
1-[6-(2-Fluoro-3-methoxy-phenyl)-1,2,3,4-tetrahydro-naphthalen-2-yl]-pyrrolidine;
[2-(6-Pyrrolidin-1-yl-5,6,7,8-tetrahydro-naphthalen-2-yl)-phenyl]-methanol;
1-(4-Methyl-5′,6′,7′,8′-tetrahydro-[1,2′]binaphthalenyl-6′-yl)-pyrrolidine;
N-[3-(6-Pyrrolidin-1-yl-5,6,7,8-tetrahydro-naphthalen-2-yl)-phenyl]-acetamide;
3-(6-Pyrrolidin-1-yl-5,6,7,8-tetrahydro-naphthalen-2-yl)-benzoic acid ethyl ester;
1-[6-(2-Benzyloxy-phenyl)-1,2,3,4-tetrahydro-naphthalen-2-yl]-pyrrolidine;
1-[6-(3-Methylsulfanyl-phenyl)-1,2,3,4-tetrahydro-naphthalen-2-yl]-pyrrolidine;
1-[6-(4-Methoxy-3-methyl-phenyl)-1,2,3,4-tetrahydro-naphthalen-2-yl]-pyrrolidine;
4-(6-Pyrrolidin-1-yl-5,6,7,8-tetrahydro-naphthalen-2-yl)-benzonitrile;
1-[6-(3,4-Dimethyl-phenyl)-1,2,3,4-tetrahydro-naphthalen-2-yl]-pyrrolidine;
1-(6-Biphenyl-2-yl-1,2,3,4-tetrahydro-naphthalen-2-yl)-pyrrolidine;
1-[6-(4-Benzyloxy-phenyl)-1,2,3,4-tetrahydro-naphthalen-2-yl]-pyrrolidine;
1-[6-(2-Fluoro-biphenyl-4-yl)-1,2,3,4-tetrahydro-naphthalen-2-yl]-pyrrolidine;
1-[6-(3,4,5-Trimethoxy-phenyl)-1,2,3,4-tetrahydro-naphthalen-2-yl]-pyrrolidine;
1-[6-(3-Benzyloxy-phenyl)-1,2,3,4-tetrahydro-naphthalen-2-yl]-pyrrolidine;
1-[6-(4-Fluoro-2-methyl-phenyl)-1,2,3,4-tetrahydro-naphthalen-2-yl]-pyrrolidine;
1-[6-(2-Ethyl-phenyl)-1,2,3,4-tetrahydro-naphthalen-2-yl]-pyrrolidine;
4-(6-Pyrrolidin-1-yl-5,6,7,8-tetrahydro-naphthalen-2-yl)-phenol;
1-[6-(2-Methylsulfanyl-phenyl)-1,2,3,4-tetrahydro-naphthalen-2-yl]-pyrrolidine;
1-[6-(4-Benzyloxy-2-fluoro-phenyl)-1,2,3,4-tetrahydro-naphthalen-2-yl]-pyrrolidine;
1-[6-(4-Isopropoxy-phenyl)-1,2,3,4-tetrahydro-naphthalen-2-yl]-pyrrolidine;
3,5-Dimethyl-1-[3-(6-pyrrolidin-1-yl-5,6,7,8-tetrahydro-naphthalen-2-yl)-phenyl]-1H-pyrazole
1-(5′,6′,7′,8′-Tetrahydro-[1,2]binaphthalenyl-6′-yl)-pyrrolidine;
1-[6-(2,3-Dihydro-benzo[1,4]dioxin-6-yl)-1,2,3,4-tetrahydro-naphthalen-2-yl]-pyrrolidine;
1-{6-[4-(1-Methoxy-ethyl)-phenyl]-1,2,3,4-tetrahydro-naphthalen-2-yl}-pyrrolidine;
1-[6-(3-Chloro-phenyl)-1,2,3,4-tetrahydro-naphthalen-2-yl]-pyrrolidine;
1-[6-(2,3-Dihydro-benzofuran-5-yl)-1,2,3,4-tetrahydro-naphthalen-2-yl]-pyrrolidine;
1-[3-(6-Pyrrolidin-1-yl-5,6,7,8-tetrahydro-naphthalen-2-yl)-phenyl]-1H-pyrazole
1-[6-(3,4-Dihydro-2H-benzo[b][1,4]dioxepin-7-yl)-1,2,3,4-tetrahydro-naphthalen-2-yl]-pyrrolidine;
1-[6-(3-Methoxy-phenyl)-1,2,3,4-tetrahydro-naphthalen-2-yl]-pyrrolidine;
2-isopropyl-5-(6-pyrrolidin-1-yl-5,6,7,8-tetrahydro-naphthalen-2-yl)-2,3-dihydro-1H-isoindole;
1-[6-(4-Chloro-phenyl)-1,2,3,4-tetrahydro-naphthalen-2-yl]-pyrrolidine;
1-[6-(5-Chloro-2-fluoro-phenyl)-1,2,3,4-tetrahydro-naphthalen-2-yl]-pyrrolidine;
1-[6-(5-Chloro-2-methoxy-phenyl)-1,2,3,4-tetrahydro-naphthalen-2-yl]-2-methyl-pyrrolidine
4-Methoxy-3-[6-(2-methyl-pyrrolidin-1-yl)-5,6,7,8-tetrahydro-naphthalen-2-yl]-benzaldehyde;
3-[6-(2-Methyl-pyrrolidin-1-yl)-5,6,7,8-tetrahydro-naphthalen-2-yl]-benzonitrile;
1-(6-Biphenyl-3-yl-1,2,3,4-tetrahydro-naphthalen-2-yl)-2-methyl-pyrrolidine;
N-tert-Butyl-2-[6-(2-methyl-pyrrolidin-1-yl)-5,6,7,8-tetrahydro-naphthalen-2-yl]-benzenesulfonamide;
2-Methyl-1-(6-phenyl-1,2,3,4-tetrahydro-naphthalen-2-yl)-pyrrolidine;
1-[6-(2,5-Dimethoxy-phenyl)-1,2,3,4-tetrahydro-naphthalen-2-yl]-2-methyl-pyrrolidine;
2-Methyl-1-(6-thianthren-1-yl-1,2,3,4-tetrahydro-naphthalen-2-yl)-pyrrolidine;
2-Methyl-1-(6-p-tolyl-1,2,3,4-tetrahydro-naphthalen-2-yl)-pyrrolidine;
2-Methyl-1-[6-(3-nitro-phenyl)-1,2,34-tetrahydro-naphthalen-2-yl]-pyrrolidine;
1-[6-(5-Fluoro-2-methoxy-phenyl)-1,2,3,4-tetrahydro-naphthalen-2-yl]-methyl-pyrrolidine;
N,N-Diisopropyl-2-methoxy-6-[6-(2-methyl-pyrrolidin-1-yl)-5,6,7,8-tetrahydro-naphthalen-2-yl]-benzamide;
1-[6-(2,6-Dimethoxy-phenyl)-1,2,3,4-tetrahydro-naphthalen-2-yl]-2-methyl-pyrrolidine;
N,N-Diisopropyl-2-[6-(2-methyl-pyrrolidin-1-yl)-5,6,78-tetrahydro-naphthalen-2-yl]-benzamide;
1-[6-(4-Fluoro-phenyl)-1,2,3,4-tetrahydro-naphthalen-2-yl]-2-methyl-pyrrolidine
1-[6-(3,5-Bis-trifluoromethyl-phenyl)-1,2,3,4-tetrahydro-naphthalen-2-yl]-2-methyl-pyrrolidine;
2-Methyl-1-(6-m-tolyl-1,2,3,4-tetrahydro-naphthalen-2-yl)-pyrrolidine;
1-{6-[4-(4-Methoxy-phenoxy)-phenyl]-1,2,3,4-tetrahydro-naphthalen-2-yl}-2-methyl-pyrrolidine;
1-[6-(3-Chloro-4-fluoro-phenyl)-1,2,3,4-tetrahydro-naphthalen-2-yl]-2-methyl-pyrrolidine;
2-Methyl-1-[6-(4-trifluoromethyl-phenyl)-1,2,3,4-tetrahydro-naphthalen-2-yl]-pyrrolidine;
2-Methyl-1-[6-(2,3,4-trimethoxy-phenyl)-1,2,3,4-tetrahydro-naphthalen-2-yl]-pyrrolidine;
1-[6-(3,5-Dichloro-phenyl)-1,2,3,4-tetrahydro-naphthalen-2-yl]-2-methyl-pyrrolidine;
2-Methyl-1-(6-o-tolyl-1,2,3,4-tetrahydro-naphthalen-2-yl)-pyrrolidine;
1-[6-(2-Methoxy-phenyl)-1,2,3,4-tetrahydro-naphthalen-2-yl]-2-methyl-pyrrolidine;
2-Methyl-1-(6-thiophen-3-yl-1,2,3,4-tetrahydro-naphthalen-2-yl)-pyrrolidine;
2-Methyl-1-[6-(4-phenoxy-phenyl)-1,2,3,4-tetrahydro-naphthalen-2-yl]-pyrrolidine;
1-[6-(3,5-Dimethyl-phenyl)-1,2,3,4-tetrahydro-naphthalen-2-yl]-2-methyl-pyrrolidine;
2-Methyl-1-(6,6,7,8-tetrahydro-[2,2′]binaphthalenyl-6-yl)-pyrrolidine;
1-[6-(2-Ethoxy-phenyl)-1,2,3,4-tetrahydro-naphthalen-2-yl]-2-methyl-pyrrolidine;
2-Methyl-1-[6-(3-trifluoromethyl-phenyl)-1,2,3,4-tetrahydro-naphthalen-2-yl]-pyrrolidine;
1-[6-(3-Ethoxy-phenyl)-1,2,3,4-tetrahydro-naphthalen-2-yl]-2-methyl-pyrrolidine;
1-[6-(3,4-Difluoro-phenyl)-1,2,3,4-tetrahydro-naphthalen-2-yl]-2-methyl-pyrrolidine;
1-[6-(3-Fluoro-phenyl)-1,2,3,4-tetrahydro-naphthalen-2-yl]-2-methyl-pyrrolidine;
2-Methyl-1-[6-(2-trifluoromethyl-phenyl)-1,2,3,4-tetrahydro-naphthalen-2-yl]-pyrrolidine;
1-[6-(4-Ethyl-phenyl)-1,2,3,4-tetrahydro-naphthalen-2-yl]-2-methyl-pyrrolidine;
1-[6-(2-Chloro-phenyl)-1,2,3,4-tetrahydro-naphthalen-2-yl]-2-methyl-pyrrolidine;
1-[6-(3,4-Dichloro-phenyl)-1,2,3,4-tetrahydro-naphthalen-2-yl]-2-methyl-pyrrolidine;
1-(6-Benzo[1,3]dioxol-5-yl-1,2,3,4-tetrahydro-naphthalen-2-yl)-2-methyl-pyrrolidine;
1-[6-(4-Ethoxy-phenyl)-1,2,3,4-tetrahydro-naphthalen-2-yl]-2-methyl-pyrrolidine;
1-[6-(2-Fluoro-phenyl)-1,2,3,4-tetrahydro-naphthalen-2-yl]-2-methyl-pyrrolidine;
{3-[6-(2-Methyl-pyrrolidin-1-yl-5,6,7,8-tetrahydro-naphthalen-2-yl]-phenyl}-ethanone;
{4-[6-(2-Methyl-pyrrolidin-1-yl)-5,6,7,8-tetrahydro-naphthalen-2-yl]-phenyl}methanol;
1-[6-(4-tert-Butyl-phenyl)-1,2,3,4-tetrahydro-naphthalen-2-yl]-2-methyl-pyrrolidine;
1-[6-(2,6-Dimethyl-phenyl)-1,2,3,4-tetrahydro-naphthalen-2-yl]-2-methyl-pyrrolidine;
2-Methyl-1-[6-(4-trifluoromethoxy-phenyl-1,2,3,4-tetrahydro-naphthalen-2-yl]-pyrrolidine;
1-[6-(2,5-Difluoro-phenyl)-1,2,3,4-tetrahydro-naphthalen-2-yl]-2-methyl-pyrrolidine;
1-[6-(4-Fluoro-3-methyl-phenyl)-1,2,3,4-tetrahydro-naphthalen-2-yl]-2-methyl-pyrrolidine;
{3-[6-(2-Methyl-pyrrolidin-1-yl)-5,6,7,8-tetrahydro-naphthalen-2-yl]-phenyl}-methanol;
1-[6-(2,4-Difluoro-phenyl)-1,2,3,4-tetrahydro-naphthalen-2-yl]-2-methyl-pyrrolidine;
1-[6-(4-Methoxy-3,5-dimethyl-phenyl)-1,2,3,4-tetrahydronaphthalen-2-yl]-2-methyl-pyrrolidine;
1-[6-(2,3-Dimethyl-phenyl)-1,2,3,4-tetrahydro-naphthalen-2-yl]-2-methyl-pyrrolidine;
2-Methyl-1-[6-(2-trifluoromethoxy-phenyl-1,2,3,4-tetrahydro-naphthalen-2-yl]-pyrrolidine;
2-Methyl-1-[6-(3-trifluoromethoxy-phenyl)-1,2,3,4-tetrahydro-naphthalen-2-yl]-pyrrolidine;
1-[6-(4-Isobutyl-phenyl)-1,2,3,4-tetrahydro-naphthalen-2-yl]-2-methyl-pyrrolidine-2-Methyl-1-[6-(2-phenoxy-phenyl)-1,2,3,4-tetrahydro-naphthalen-2-yl]-pyrrolidine;
1-[6-(3,5-Difluoro-phenyl)-1,2,3,4-tetrahydro-naphthalen-2-yl]-2-methyl-pyrrolidine;
4-[6-(2-Methyl-pyrrolidin-1-yl)-5,6,7,8-tetrahydro-naphthalen-2-yl]-benzoic acid isopropyl ester;
1-[6-(2,5-Dimethyl-phenyl)-1,2,3,4-tetrahydro-naphthalen-2-yl]-2-methyl-pyrrolidine;
1-{2-[6-(2-Methyl-pyrrolidin-1-yl)-5,6,7,8-tetrahydro-naphthalen-2-yl]-phenyl}-ethanone;
4-[6-(2-Methyl-pyrrolidin-1-yl)-5,6,7,8-tetrahydro-naphthalen-2-yl]-benzamide;
3-[6-(2-Methyl-pyrrolidin-1-yl)-5,6,7,8-tetrahydro-naphthalen-2-yl]-phenol;
2-Methyl-1-(4-methyl-5′,6′,7′,8′-tetrahydro-[1,2′]binaphthalenyl-6′-yl)-pyrrolidine;
1-[6-(2-Benzyloxy-phenyl)-1,2,3,4-tetrahydro-naphthalen-2-yl]-2-methyl-pyrrolidine;
3-[6-(2-Methyl-pyrrolidin-1-yl)-5,6,7,8-tetrahydro-naphthalen-2-yl]-pyridine;
{2-[6-(2-Methyl-pyrrolidin-1-yl)-5,6,7,8-tetrahydro-naphthalen-2-yl]-phenyl}-methanol;
1-[6-(2-Fluoro-3-methoxy-phenyl)-1,2,3,4-tetrahydro-naphthalen-2-yl]-2-methyl-pyrrolidine;
3-[6-(2-Methyl-pyrrolidin-1-yl)-5,6,7,8-tetrahydro-naphthalen-2-yl]-benzoic acid ethyl ester;
1-(6-Furan-3-yl-1,2,3,4-tetrahydro-naphthalen-2-yl)-2-methyl-pyrrolidine;
2-Methyl-1-[6-(3-methylsulfanyl-phenyl)-1,2,3,4-tetrahydro-naphthalen-2-yl]-pyrrolidine;
1-[6-(4-Methoxy-3-methyl-phenyl)-1,2,3,4-tetrahydro-naphthalen-2-yl]-2-methyl-pyrrolidine;
{4-[6-(2-Methyl-pyrrolidin-1-yl)-5,6,7,8-tetrahydro-naphthalen-2-yl]-phenyl}-carbamic acid benzyl ester;
2-Methyl-1-[6-(5-methyl-furan-2-yl)-1,2,3,4-tetrahydro-naphthalen-2-yl]-pyrrolidine;
4-[6-(2-Methyl-pyrrolidin-1-yl)-5,6,7,8-tetrahydro-naphthalen-2-yl]-benzoic acid ethyl ester;
2-Methoxy-5-[6-(2-methyl-pyrrolidin-1-yl)-5,6,7,8-tetrahydro-naphthalen-2-yl]-pyridine;
1-(6-Biphenyl-2-yl-1,2,3,4-tetrahydro-naphthalen-2-yl)-2-methyl-pyrrolidine;
N-{3-[6-(2-Methyl-pyrrolidin-1-yl)-5,6,7,8-tetrahydro-naphthalen-2-yl]-phenyl}-acetamide,
1-[6-(2,6-Dichloro-phenyl)-1,2,3,4-tetrahydro-naphthalen-2-yl]-2-methyl-pyrrolidine;
1-(6-Dibenzothiophen-1-yl-1,2,3,4-tetrahydro-naphthalen-2-yl)-2-methyl-pyrrolidine;
1-[6-(2-Methoxy-5-methyl-phenyl)-1,2,3,4-tetrahydro-naphthalen-2-yl]-2-methyl-pyrrolidine;
2-Methyl-1-[6-(3,4,5-trimethoxy-phenyl)-1,2,3,4-tetrahydro-naphthalen-2-yl]-pyrrolidine;
1-[6-(4-Benzyloxy-3-fluoro-phenyl)-1,2,3,4-tetrahydro-naphthalen-2-yl]-2-methyl-pyrrolidine;
1-[6-(3,4-Dimethyl-phenyl)-1,2,3,4-tetrahydro-naphthalen-2-yl]-2-methyl-pyrrolidine;
4-[6-(2-Methyl-pyrrolidin-1-yl)-5,6,7,8-tetrahydro-naphthalen-2-yl]-benzoic acid benzyl ester;
1-[6-(2-Fluoro-biphenyl-4-yl)-1,2,3,4-tetrahydro-naphthalen-2-yl]-2-methyl-pyrrolidine;
4-[6-(2-Methyl-pyrrolidin-1-yl)-5,6,7,8-tetrahydro-naphthalen-2-yl]-benzonitrile;
1-[6-(4-Isopropyl-phenyl)-1,2,34-tetrahydro-naphthalen-2-yl]-2-methyl-pyrrolidine;
1-[2,3-Difluoro-phenyl)-1,2,3,4-tetrahydro-naphthalen-2-yl]-2-methyl-pyrrolidine;
1-[6-(5-Isopropyl-2-methoxy-phenyl)-1,2,3,4-tetrahydro-naphthalen-2-yl]-2-methyl-pyrrolidine;
2-Methyl-1-[6-(4-pentyl-phenyl)-1,2,3,4-tetrahydro-naphthalen-2-yl]-pyrrolidine;
1-[6-(2,3-Dichloro-phenyl)-1,2,3,4-tetrahydronaphthalen-2-yl]-2-methyl-pyrrolidine;
1-[6-(4-Methanesulfonyl-phenyl)-1,2,3,4-tetrahydro-naphthalen-2-yl]-2-methyl-pyrrolidine;
4-[6-(2-Methyl-pyrrolidin-1-yl)-5,6,7,8-tetrahydro-naphthalen-2-yl]-phenol;
2-Methyl-1-[6-(2-methylsulfanyl-phenyl)-1,2,3,4-tetrahydro-naphthalen-2-yl]-pyrrolidine;
1-[6-(2,4-Bis-trifluoromethyl-phenyl)-1,2,3,4-tetrahydro-naphthalen-2-yl]-2-methyl-pyrrolidine;
1-[6-(3-Benzyloxy-phenyl)-1,2,3,4-tetrahydro-naphthalen-2-yl]-2-methyl-pyrrolidine;
1-[6-(4-Fluoro-2-methyl-phenyl)-1,2,3,4-tetrahydro-naphthalen-2-yl]-2-methyl-pyrrolidine;
3-[6-(2-Methyl-pyrrolidin-1-yl)-5,6,7,8-tetrahydro-naphthalen-2-yl]-benzoic acid;
1-[6-(2-Ethyl-phenyl)-1,2,3,4-tetrahydro-naphthalen-2-yl]-2-methyl-pyrrolidine;
1-[6-(4-Benzyloxy-phenyl)-1,2,3,4-tetrahydro-naphthalen-2-yl]-2-methyl-pyrrolidine;
1-[6-(4-Benzyloxy-2-fluoro-phenyl)-1,2,34-tetrahydro-naphthalen-2-yl]-2-methyl-pyrrolidine;
1-[6-(4-Butyl-phenyl)-1,2,3,4-tetrahydro-naphthalen-2-yl]-2-methyl-pyrrolidine;
2-Methyl-1-(5′,6′,7′,8′-tetrahydro-[1,2′]binaphthalenyl-6′-yl)-pyrrolidine;
1-[6-(4-Chloro-phenyl)-1,2,3,4-tetrahydro-naphthalen-2-yl]-2-methyl-pyrrolidine;
1-[6-(4-Ethanesulfonyl-phenyl)-1,2,3,4-tetrahydro-naphthalen-2-yl]-2-methyl-pyrrolidine;
1-[6-(2,4-Dichloro-phenyl)-1,2,3,4-tetrahydronaphthalen-2-yl]-2-methyl-pyrrolidine;
1-{3-[6-(2-Methyl-pyrrolidin-1-yl)-5,6,7,8-tetrahydro-naphthalen-2-yl]-phenyl}-1H-pyrazole;
3-[6-(2-Methyl-pyrrolidin-1-yl)-5,6,7,8-tetrahydro-naphthalen-2-yl]-benz-amide;
1-(6-Dibenzofuran-4-yl-1,2,3,4-tetrahydro-naphthalen-2-yl)-2-methyl-pyrrolidine;
1-[6-(4-Chloro-phenyl)-1,2,3,4-tetrahydro-naphthalen-2-yl]-2-methyl-pyrrolidine;
1-[6-(4-Isopropoxy-phenyl)-1,2,3,4-tetrahydro-naphthalen-2-yl]-2-methyl-pyrrolidine;
3-Chloro-4-[6-(2-methyl-pyrrolidin-1-yl)-5,6,7,8-tetrahydro-naphthalen-2-yl]-pyridine;
1-[6-(3-Methoxy-phenyl)-1,2,3,4-tetrahydro-naphthalen-2-yl]-2-methyl-pyrrolidine;
2-Methyl-1-[6-(4-methyl-3-nitro-phenyl)-1,2,3,4-tetrahydro-naphthalen-2-yl]-pyrrolidine;
2-Methyl-1-(6-methyl-1,2,3,4-tetrahydro-naphthalen-2-yl)-pyrrolidine;
1-[6-(3,4-Dihydro-2H-benzo[b][1,4]dioxepin-7-yl)-1,2,3,4-tetrahydro-naphthalen-2-yl]-2-methyl-pyrrolidine;
1-[6-(2,3-Dihydro-benzo[1,4]dioxin-6-yl)-1,2,3,4-tetrahydro-naphthalen-2-yl]-2-methyl-pyrrolidine;
3,5-Dimethyl-1-{3-[6-(2-methyl-pyrrolidin-1-yl)-5,6,7,8-tetrahydro-naphthalen-2-yl]-phenyl}-1H-pyrazole;
5-[6-(2-Methyl-pyrrolidin-1-yl)-5,6,7,8-tetrahydro-naphthalen-2-yl]-pyrimidine;
1-[6-(4-Methoxy-phenyl)-1,2,3,4-tetrahydro-naphthalen-2-yl]-2-methyl-pyrrolidine;
1-[6-(4-Methoxy-2,6-dimethyl-phenyl)-1,2,3,4-tetrahydro-naphthalen-2-yl]-2-methyl-pyrrolidine;
1-[6-(2,3-Dihydro-benzofuran-5-yl)-1,2,3,4-tetrahydro-naphthalen-2-yl]-2-methyl-pyrrolidine;
1-[6-(4-Ethylsulfanyl-phenyl)-1,2,3,4-tetrahydro-naphthalen-2-yl]-2-methyl-pyrrolidine;
1-{6-[4-(1-Methoxy-ethyl)-phenyl]-1,2,3,4-tetrahydro-naphthalen-2-yl}-2-methyl-pyrrolidine;
1-[6-(2-Chloro-5-fluoro-phenyl)-1,2,3,4-tetrahydro-naphthalen-2-yl]-2-methyl-pyrrolidine,
1-Benzenesulfonyl-3-[6-(2-methyl-pyrrolidin-1-yl)-5,6,7,8-tetrahydro-naphthalen-2-yl]-1H-indole;
1-[6-(5-Chloro-2-fluoro-phenyl)-1,2,3,4-tetrahydro-naphthalen-2-yl]-2-methyl-pyrrolidine;
2-Isopropyl-5-[6-(2-methyl-pyrrolidin-1-yl-5,6,7,8-tetrahydro-naphthalen-2-yl]-2,3-dihydro-1H-isoindole;
2-[Ethyl-(6-pyridin-3-yl-1,2,3,4-tetrahydro-naphthalen-2-yl)-amino]-ethanol;
[1-(6-Pyridin-3-yl-1,2,3,4-tetrahydro-naphthalen-2-yl)-piperidin-3-yl]-methanol;
3-(6-Azetidin-1-yl-5,6,7,8-tetrahydro-naphthalen-2-yl)-pyridine;
4-(6-Azetidin-1-yl-5,6,7,8-tetrahydro-naphthalen-2-yl)-benzamide;
1-[6-(6-Methoxy-2-methyl-pyridin-3-yl)-1,2,3,4-tetrahydro-naphthalen-2-yl]-pyrrolidine-2-carboxylic acid dimethylamide;
1-[6-(4-Methanesulfonyl-phenyl)-1,2,3,4-tetrahydro-naphthalen-2-yl]-pyrrolidine-2-carboxylic acid dimethylamide;
1-[6-(4-Methanesulfonyl-phenyl)-1,2,3,4-tetrahydro-naphthalen-2-yl]-azetidine;
1-[6-(4-Ethanesulfonyl-phenyl)-1,2,3,4-tetrahydro-naphthalen-2-yl]-pyrrolidine-2-carboxylic acid dimethylamide;
1-[6-(4-Methanesulfonyl-phenyl)-1,2,3,4-tetrahydro-naphthalen-2-yl]-2-methoxymethyl-pyrrolidine;
1-[6-(4-Ethanesulfonyl-phenyl)-1,2,3,4-tetrahydro-naphthalen-2-yl]-2-methoxymethyl-pyrrolidine;
1-[6-(4-Ethanesulfonyl-phenyl)-1,2,3,4-tetrahydro-naphthalen-2-yl]-azetidine;
1-[6-(4-Methoxy-pyridin-3-yl)-1,2,3,4-tetrahydro-naphthalen-2-yl]-pyrrolidine-2-carboxylic acid dimethylamide;
1-[6-(3-Methanesulfonyl-phenyl)-1,2,3,4-tetrahydro-naphthalen-2-yl]-2-methoxymethyl-pyrrolidine;
2-Methoxy-5-[6-(2-methoxymethyl-pyrrolidin-1-yl)-5,6,7,8-tetrahydro-naphthalen-2-yl]-pyridine;
3-[6-(2-Methoxymethyl-pyrrolidin-1-yl)-5,6,7,8-tetrahydro-naphthalen-2-yl]-N,N-dimethyl-benzamide;
1-[6-(3-Methanesulfonyl-phenyl)-1,2,3,4-tetrahydro-naphthalen-2-yl]-azetidine;
4-[6-(2-Methoxymethyl-pyrrolidin-1-yl)-5,6,7,8-tetrahydro-naphthalen-2-yl]-N,N-dimethyl-benzamide;
3-[6-(2-Methoxymethyl-pyrrolidin-1-yl)-5,6,7,8-tetrahydro-naphthalen-2-yl]-benzamide;
4-[6-(2-Methoxymethyl-pyrrolidin-1-yl)-5,6,7,8-tetrahydro-naphthalen-2-yl]-N-methyl-benzamide;
1-[6-(4-Dimethylcarbamoyl-phenyl)-1,2,314-tetrahydro-naphthalen-2-yl]-pyrrolidine-2-carboxylic acid dimethylamide;
3-[6-(2-Phenyl-pyrrolidin-1-yl)-5,6,7,8-tetrahydro-naphthalen-2-yl]-benzamide;
3-(6-Azetidin-1-yl-5,6,7,8-tetrahydro-naphthalen-2-yl)-benzamide;
1-Isopropyl-4-[6-(6-methoxy-2-methyl-pyridin-3-yl)-1,2,3,4-tetrahydro-naphthalen-2-yl]-piperazine;
1-[6-(4-Methanesulfonyl-phenyl)-1,2,3,4-tetrahydro-naphthalen-2-yl]-piperidine;
1-isopropyl-4-[6-(4-methanesulfonyl-phenyl)-1,2,3,4-tetrahydro-naphthalen-2-yl]-piperazine;
1-[6-(4-Methoxy-2-methyl-pyridin-3-yl)-1,2,3,4-tetrahydro-naphthalen-2-yl]-piperidin-3-ol;
1-[6-(4-Ethanesulfonyl-phenyl)-1,2,3,4-tetrahydro-naphthalen-2-yl]-4-isopropyl-piperazine;
1-[6-(4-Methanesulfonyl-phenyl)-1,2,3,4-tetrahydro-naphthalen-2-yl]-piperidin-3-ol;
1-[6-(4-Ethanesulfonyl-phenyl)-1,2,3,4-tetrahydro-naphthalen-2-yl]-piperidine;
4-[6-(6-Methoxy-2-methyl-pyridin-3-yl-1,2,3,4-tetrahydro-naphthalen-2-yl]-thiomorpholine;
[6-(4-Ethanesulfonyl-phenyl)-1,2,3,4-tetrahydro-naphthalen-2-yl]-diethyl-amine;
1-[6-(4-Ethanesulfonyl-phenyl)-1,2,3,4-tetrahydro-naphthalen-2-yl]-piperidin-3-ol;
6-Methoxy-2-methyl-3-(6-piperidin-1-yl-5,6,7,8-tetrahydro-naphthalen-2-yl)-pyridine;
Diethyl-[6-(4-methanesulfonyl-phenyl)-1,2,3,4-tetrahydro-naphthalen-2-yl]-amine;
4-[6-(4-Methanesulfonyl-phenyl)-1,2,3,4-tetrahydro-naphthalen-2-yl]-3-methyl-morpholine;
Diethyl-(6-pyrimidin-5-yl-1,2,34-tetrahydro-naphthalen-2-yl)-amine;
4-[6-(4-Ethanesulfonyl-phenyl)-1,2,3,4-tetrahydro-naphthalen-2-yl]-thiomorpholine;
4-[6-(6-Methoxy-2-methyl-pyridin-3-yl)-1,2,3,4-tetrahydro-naphthalen-2-yl]-3-methyl-morpholine;
4-[6-(4-Ethanesulfonyl-phenyl)-1,2,3,4-tetrahydro-naphthalen-2-yl]-3-methyl-morpholine;
1-Isopropyl-4-[6-(6-methoxy-pyridin-3-yl)-1,2,3,4-tetrahydro-naphthalen-2-yl]-piperazine;
4-[6-(6-Methoxy-pyridin-3-yl)-1,2,3,4-tetrahydro-naphthalen-2-yl]-thiomorpholine;
5-[6-(4-Isopropyl-piperazin-1-yl)-5,6,7,8-tetrahydro-naphthalen-2-yl]-pyrimidine;
4-[6-(4-Isopropyl-piperazin-1-yl)-5,6,7,8-tetrahydro-naphthalen-2-yl]-benzamide;
1-(6-Pyrimidin-5-yl-1,2,3,4-tetrahydro-naphthalen-2-yl)-piperidin-3-ol;
1-Isopropyl-4-[6-(3-methanesulfonyl-phenyl)-1,2,3,4-tetrahydro-naphthalen-2-yl]-piperazine;
1-[6-(6-Methoxy-pyridin-3-yl)-1,2,3,4-tetrahydro-naphthalen-2-yl]-piperidin-3-o;
5-(6-Piperidin-1-yl-5,6,7,8-tetrahydro-naphthalen-2-yl)-pyrimidine;
4-[6-(3-Hydroxy-piperidin-1-yl)-5,6,7,8-tetrahydro-naphthalen-2-yl]-benzamide;
2-Methoxy-5-(6-piperidin-1-yl-5,6,7,8-tetrahydro-naphthalen-2-yl)-pyridine;
1-[6-(3-Methanesulfonyl-phenyl)-1,2,3,4-tetrahydro-naphthalen-2-yl]-piperidin-3-ol,
4-(6-Thiomorpholin-4-yl-5,6,7,8-tetrahydro-naphthalen-2-yl)-benzamide;
3-[6-(4-isopropyl-piperazin-1-yl)-5,6,7,8-tetrahydro-naphthalen-2-yl]-N,N-dimethyl-benzamide;
3-[6-(3-Hydroxy-piperidin-1-yl)-5,6,7,8-tetrahydro-naphthalen-2-yl]-N,N-dimethyl-benzamide;
3-[6-(2-Isopropyl-pyrrolidin-1-yl)-5,6,7,8-tetrahydro-naphthalen-2-yl]-6-methoxy-2-methyl-pyridine;
1-[6-(4-Ethanesulfonyl-phenyl)-1,2,3,4-tetrahydro-naphthalen-2-yl]-2-methyl-pyrrolidine;
2-[6-(4-Ethanesulfonyl-phenyl)-1,2,3,4-tetrahydro-naphthalen-2-yl]-2,3-dihydro-1H-isoindole;
2-[6-(6-Methoxy-2-methyl-pyridin-3-yl)-1,2,3,4-tetrahydro-naphthalen-2-yl]-2,3-dihydro-1H-isoindole;
1-[6-(4-Ethanesulfonyl-phenyl)-1,2,3,4-tetrahydro-naphthalen-2-yl]-2-isopropyl-pyrrolidine;
5-[6-(2-isopropyl-pyrrolidin-1-yl)-5,6,7,8-tetrahydro-naphthalen-2-yl]-pyrimidine;
4-[6-(4-Methanesulfonyl-phenyl)-1,2,3,4-tetrahydro-naphthalen-2-yl]-morpholine;
4-[6-(4-Ethanesulfonyl-phenyl)-1,2,3,4-tetrahydro-naphthalen-2-yl]-morpholine;
4-[6-(6-Methoxy-2-methyl-pyridin-3-yl)-1,2,3,4-tetrahydro-naphthalen-2-yl]-morpholine;
4-(6-Pyrimidin-5-yl-1,2,3,4-tetrahydro-naphthalen-2-yl)-morpholine;
3-[6-(2-Isopropyl-pyrrolidin-1-yl)-5,6,7,8-tetrahydro-naphthalen-2-yl]-N,N-dimethyl-benzamide;
1-[6-(4-Methanesulfonyl-phenyl)-1,2,3,4-tetrahydro-naphthalen-2-yl]-pyrrolidin-3-ol;
4-[6-(1,3-Dihydro-isoindol-2-yl)-5,6,7,8-tetrahydro-naphthalen-2-yl]-N,N-dimethyl-benzamide;
2-Methoxy-5-[6-(3-phenyl-pyrrolidin-1-yl)-5,6,7,8-tetrahydro-naphthalen-2-yl]-pyridine;
3-[6-(1,3-Dihydro-isoindol-2-yl)-5,6,7,8-tetrahydro-naphthalen-2-yl]-N,N-dimethyl-benzamide;
4-[6-(6-Methoxy-pyridin-3-yl)-1,2,3,4-tetrahydro-naphthalen-2-yl]-morpholine;
3-[6-(3-(Phenyl-pyrrolidin-1-yl)-5,6,7,8-tetrahydro-naphthalen-2-yl]-benzamide;
5-[6-(2-Isopropyl-pyrrolidin-1-yl)-5,6,7,8-tetrahydro-naphthalen-2-yl]-2-methoxy-pyridine,
4-[6-(Benzyl-methyl-amino)-5,6,7,8-tetrahydro-naphthalen-2-yl]-N,N-dimethyl-benzamide;
4-[6-(3-Methanesulfonyl-phenyl)-1,2,3,4-tetrahydro-naphthalen-2-yl]-morpholine;
1-[6-(3-Methanesulfonyl-phenyl)-1,2,3,4-tetrahydro-naphthalen-2-yl]-3-phenyl-pyrrolidine;
and pharmaceutically acceptable salts thereof.

11. A compound of formula I, according to claim 1, selected from the group consisting of
3-(6-Pyrrolidin-1-yl-5,6,7,8-tetrahydro-naphthalen-2-yl)-benzamide:
Azetidin-1-yl-[4-(6-pyrrolidin-1-yl-5,6,7,8-tetrahydro-naphthalen-2-yl)-phenyl]-methanone;
N-Cyclobutyl-4-(6-pyrrolidin-1-yl-5,6,7,8-tetrahydro-naphthalen-2-yl)-benzamide;
N-Isobutyl-3-(6-pyrrolidin-t-yl-5,6,7,8-tetrahydro-naphthalen-2-yl)-benzamide;
4-(6-Pyrrolidin-1-yl-5,6,7,8-tetrahydro-naphthalen-2-yl)-pyridine;
N-(2-Methoxy-ethyl)-3-(6-pyrrolidin-1-yl-5,6,7,8-tetrahydro-naphthalen-2-yl)-benzamide methanone;
N-(2-Hydroxy-ethyl)-N-methyl-3-(6-pyrrolidin-1-yl-5,6,7,8-tetrahydro-naphthalen-2-yl)-benzamide;
N-Cyclopropyl-3-(6-pyrrolidin-1-yl-5,6,7,8-tetrahydro-naphthalen-2-yl)-benzamide;
3-(6-Pyrrolidin-1-yl-5,6,7,8-tetrahydro-naphthalen-2-yloxy)-benzamide;
5-(6-Pyrrolidin-1-yl-5,6,7,8-tetrahydro-naphthalen-2-yl)-oxazole;
1-[6-(4-Methanesulfonyl-phenoxy)-1,2,3,4-tetrahydro-naphthalen-2-yl]-pyrrolidine;
N-(2-Hydroxy-ethyl)-N-methyl-(6-pyrrolidin-1-yl-5,6,7,8-tetrahydronaphthalen-2-yl)-benzamide;
4-(6-Pyrrolidin-1-yl-5,6,7,8-tetrahydro-naphthalen-2-yl)-N-(tetrahydro-pyran-4-yl)-benzamide;
N-(2-Methoxy-ethyl)-4-(6-pyrrolidin-1-yl-5,6,7,8-tetrahydro-naphthalen-2-yl)-benzamide;
N-Isobutyl-4-(6-pyrrolidin-t-yl-5,6,7,8-tetrahydro-naphthalen-2-yl)-benzamide;
1-[6-(4-Methoxy-phenoxy)-1,2,3,4-tetrahydro-naphthalen-2-yl]-pyrrolidine;
N,N-dimethyl-4-(6-pyrrolidin-1-yl-5,6,7,8-tetrahydro-naphthalen-2-yl)-benzamide;
Azetidin-1-yl-[4-(6-pyrrolidin-1-yl-5,6,7,8-tetrahydro-naphthalen-2-yl)-phenyl]-methanone;
N-Ethyl-N-methyl-4-(6-pyrrolidin-1-yl-5,6,7,8-tetrahydro-naphthalen-2-yl)-benzamide;
(+)-N-Ethyl-N-methyl-4-(6-pyrrolidin-1-yl-5,6,7,8-tetrahydro-naphthalen-2-yl)-benzamide;
(−)-N-Ethyl-N-methyl-4-(6-pyrrolidin-1-yl-5,6,7,8-tetrahydro-naphthalen-2-yl)-benzamide;
2-Methoxy-5-(6-pyrrolidin-1-yl-5,6,7,8-tetrahydro-naphthalen-2-yl)-pyridine;
3-(6-Pyrrolidin-1-yl-5,6,7,8-tetrahydro-naphthalen-2-yl)-pyridine;
2-Methoxy-3-(6-pyrrolidin-1-yl-5,6,7,8-tetrahydro-naphthalen-2-yl)-pyridine
6-Methoxy-2-methyl-3-(6-pyrrolidin-1-yl-5,6,7,8-tetrahydro-naphthalen-2-yl)-pyridine;
N-Isopropyl-4-(6-pyrrolidin-1-yl-5,6,7,8-tetrahydro-naphthalen-2-yl)-benzamide,
N-Cyclobutyl-4-(6-pyrrolidin-1-yl-5,6,7,8-tetrahydro-naphthalen-2-yl)-benzamide;
4-(6-Pyrrolidin-1-yl-5,6,7,8-tetrahydro-naphthalen-2-yl)-phenol;
1-[6-(4-Methoxy-2,6-dimethyl-phenyl)-1,2,3,4-tetrahydro-naphthalen-2-yl]-pyrrolidine;
1-[6-(4-Methanesulfonyl-phenyl)-1,2,3,4-tetrahydro-naphthalen-2-yl]-pyrrolidine;
(R)-1-[6-(4-Methanesulfonyl-phenyl)-1,2,3,4-tetrahydro-naphthalen-2-yl]-pyrrolidine;
(S)-1-[6-(4-Methanesulfonyl-phenyl)-1,2,3,4-tetrahydro-naphthalen-2-yl]-pyrrolidine;
3-(6-Pyrrolidin-1-yl-5,6,7,8-tetrahydro-naphthalen-2-yl)-benzamide;
(S)-3-(6-Pyrrolidin-1-yl-5,6,7,8-tetrahydro-naphthalen-2-yl)-benzamide;
(R)-3-(6-Pyrrolidin-1-yl-5,6,78-tetrahydro-naphthalen-2-yl)-benzamide;
1-[6-(4-Methoxy-phenyl)-1,2,3,4-tetrahydro-naphthalen-2-yl]-pyrrolidine;
(R)-1-[6-(4-Methoxy-phenyl)-1,2,3,4-tetrahydro-naphthalen-2-yl]-pyrrolidine;
(S)-1-[6-(4-Methoxy-phenyl)-1,2,3,4-tetrahydro-naphthalen-2-yl]-pyrrolidine;
1-Isopropyl-4-[6-(4-methoxy-phenyl)-1,2,3,4-tetrahydro-naphthalen-2-yl]-piperazine;
(S)-(−)-1-[6-(4-Chloro-phenyl)-1,2,3,4-tetrahydro-naphthalen-2-yl]-pyrrolidine;
(R)-(+)-1-[6-(4-Chloro-phenyl)-1,2,3,4-tetrahydro-naphthalen-2-yl]-pyrrolidine;
3-Fluoro-1-[6-(4-methoxy-phenyl)-1,2,3,4-tetrahydro-naphthalen-2-yl]-pyrrolidine;
1-[4-(6-Pyrrolidin-1-yl-5,6,78-tetrahydro-naphthalen-2-yl)-phenyl]-ethanone;
3,4-Difluoro-1-[6-(4-methoxy-phenyl)-1,2,3,4-tetrahydro-naphthalen-2-yl]-pyrrolidine;
1-[6-(4-Methoxy-phenyl)-1,2,3,4-tetrahydro-naphthalen-2-yl]-2-methyl-pyrrolidine;
(R,R)-1-[6-(4-Methoxy-phenyl)-1,2,3,4-tetrahydro-naphthalen-2-yl]-2-methyl-pyrrolidine;
(S,R)-1-[6-(4-Methoxy-phenyl)-1,2,3,4-tetrahydro-naphthalen-2-yl]-2-methyl-pyrrolidine;
(R)-1-(6-bromo-1,2,3,4-tetrahydronaphthalen-2-yl)pyrrolidine;
(S)-1-(6-bromo-1,2,3,4-tetrahydronaphthalen-2-yl)pyrrolidine;
(R,R)-1-(6-Bromo-1,2,3,4-tetrahydro-naphthalen-2-yl)-2-methyl-pyrrolidine;
(S,R)-1-(6-Bromo-1,2,3,4-tetrahydro-naphthalen-2-yl)-2-methyl-pyrrolidine;
(R,S)-1-(6-Bromo-1,2,3,4-tetrahydro-naphthalen-2-yl)-2-methyl-pyrrolidine;
(S,S)-1-(6-Bromo-1,2,3,4-tetrahydro-naphthalen-2-yl)-2-methyl-pyrrolidine;
(R)-N,N-Dimethyl-(6-pyrrolidin-1-yl-5,6,7,8-tetrahydro-naphthalen-2-yl)-benzamide;
(R)-N,N-Dimethyl-3-(6-pyrrolidin-1-yl-5,6,7,8-tetrahydro-naphthalen-2-yl)-benzamide;
(S,R)-3-[6-(2-Methyl-pyrrolidin-1-yl)-5,6,7,8-tetrahydro-naphthalen-2-yl]-pyridine;
(R,R)-3-[6-(2-Methyl-pyrrolidin-1-yl)-5,6,7,8-tetrahydro-naphthalen-2-yl]-pyridine;
1-[6-(3,4-Dimethoxy-phenyl)-1,2,3,4-tetrahydro-naphthalen-2-yl]-2-methyl-pyrrolidine;
1-[6-(3-Fluoro-4-methoxy-phenyl)-1,2,3,4-tetrahydro-naphthalen-2-yl]-2-methyl-pyrrolidine;
N-Methyl-4-(6-pyrrolidin-1-yl-5,6,7,8-tetrahydro-naphthalen-2-yl)-benzamide;
4-[6-(2-Methyl-pyrrolidin-1-yl)-5,6,7,8-tetrahydro-naphthalen-2-yl]-benzamide;
3-[6-(2-Methyl-pyrrolidin-1-yl)-5,6,7,8-tetrahydro-naphthalen-2-yl]-benzamide;
(R,R)-3-[6-(2-Methyl-pyrrolidin-1-yl)-5,6,7,8-tetrahydro-naphthalen-2-yl]-benzamide;
(S,R)—[6-(2-Methyl-pyrrolidin-1-yl)-5,6,7,8-tetrahydro-naphthalen-2-yl]-benzamide;
1-[6-(4-Methanesulfonyl-phenyl)-1,2,3,4-tetrahydro-naphthalen-2-yl]-2-methyl-pyrrolidine;
and pharmaceutically acceptable salts thereof.

12. A pharmaceutical composition for treating a disorder or condition that may be treated by antagonizing histamine-3 receptors, the composition comprising a compound of formula i as described in claim 1, and optionally a pharmaceutically acceptable carrier.

13. A method of treatment of a disorder or condition that may be treated by antagonizing histamine-3 receptors, the method comprising administering to a mammal in need of such treatment a compound of formula I as described in claim t.

14. The method of claim 13 selected from the group consisting of depression, mood disorders, schizophrenia, anxiety disorders, cognitive disorders, Alzheimer's disease, attention-deficit disorder, attention-deficit hyperactivity disorder, psychotic, disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I as described in claim 1.

15. The method of claim 14, wherein the disorder or condition is selected from the group consisting of anxiety disorders, attention-deficit hyperactivity disorder, attention-deficit disorder, respiratory diseases, obesity, cognitive disorders, and psychotic disorders.

16. The method of claim 15, wherein the disorder or condition is a respiratory disease selected from the group consisting of adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

17. A pharmaceutical composition for treating allergic rhinitis, nasal congestion or allergic congestion comprising:
(a) an H3 receptor antagonist compound of formula I; or a pharmaceutically acceptable salt thereof;
(b) an H1 receptor antagonist or a pharmaceutically acceptable salt thereof; and
(c) a pharmaceutically acceptable carrier,
wherein the active ingredients (a) and (b) above are present in amounts that render the composition effective in treating allergy rhinitis, nasal congestion or allergic congestion.

18. The pharmaceutical composition according to claim 16, wherein said Hi receptor antagonist is selected from the group consisting of cetirizine chlorpheniramine, loratidine, fexofenadine, and desloratadine.

19. A pharmaceutical composition for treating attention-deficit disorder, attention-deficit hyperactivity disorder, depression, mood disorders, or cognitive disorders comprising:
a) an H3 receptor antagonist compound of Formula I or a pharmaceutically acceptable salt thereof;
b) a neurotransmitter re-uptake blocker or a pharmaceutically acceptable salt thereof;
c) a pharmaceutically acceptable carrier,
wherein the active ingredients (a) and (b) above are present in amounts that render the composition effective in treating depression, mood disorders, and cognitive disorders.

20. The pharmaceutical composition according to claim 19, wherein the neurotransmitter re-uptake blocker is selected from the group consisting of sertraline, fluoxetine and paroxetine.
